(19) United States
(12) Patent Application Publication
(10) Pub. No.: US 2020/0206344 A1
(43) Pub. Date: Jul. 2, 2020

(54) METHODS FOR MODULATING THE INTERACTION BETWEEN EWS-FLI1 AND BAF COMPLEXES

(71) Applicants: Dana-Farber Cancer Institute, Inc., Boston, MA (US); The General Hospital Corporation, Boston, MA (US)

(72) Inventors: Cigall Kadoch, Newton, MA (US); Gabriel Sandoval, Brighton, MA (US); Miguel Rivera, Charlestown, MA (US); Gaylor Boulay, Boston, MA (US)

(21) Appl. No.: 16/640,942

(22) PCT Filed: Aug. 20, 2018
(86) PCT No.: PCT/US18/00339

§ 371 Date: Feb. 21, 2020

Related U.S. Application Data
(60) Provisional application No. 62/550,103, filed on Aug. 25, 2017.
(60) Provisional application No. 62/548,334, filed on Aug. 21, 2017.

(51) Int. Cl. A61K 39/395; C12N 15/113; A61K 39/00; A61K 45/06; C07K 16/18; C12Q 1/6886; G01N 33/574; A61P 35/00; A61K 31/713

(52) U.S. Cl. CPC A61K 39/3955; G01N 2500/02; A61K 39/0011; A61K 45/06; C07K 16/18; C12Q 1/6886; G01N 33/57484; A61P 35/00; A61K 31/713; C12N 2310/14; C07K 2317/24; C07K 2317/21; C07K 2317/76; C12Q 2600/136; C12Q 2600/158; C12N 15/113

(57) ABSTRACT

The present invention is based, in part, on the novel discovery of the interaction between EWS-FLI1 and BAF complexes, and methods of modulating same to treat cancer, including Ewing Sarcoma.

### CROSS-REFERENCE TO RELATED APPLICATIONS

This application claims the benefit of U.S. Provisional Application No. 62/548,334, filed on 21 Aug. 2017; and U.S. Provisional Application No. 62/550,103, filed on 25 Aug. 2017; the entire contents of each of said applications are incorporated herein in their entirety by this reference.

### STATEMENT OF RIGHTS

This invention was made with government support under grant number 1DP2CA195762-01, awarded by The National Institutes of Health. The government has certain rights in the present invention.

### BACKGROUND OF THE INVENTION

Temporal and spatial regulation of gene expression plays a fundamental role in directing cell identity and proliferation in both normal tissues and in human disease. The striking number of genetic alterations in genes encoding transcription factors, chromatin modifiers, and histones that have been uncovered in recent whole-exome sequencing efforts have further highlighted the importance of gene regulation in cancer (Lander, 2011). While these alterations can have profound consequences on cancer-specific gene expression, their precise mechanisms of action, in most cases, remain poorly understood.

In contrast to most adult tumor types, pediatric cancers are often driven by a limited number of genetic alterations (Lawrence et al., 2013). Pathognomonic chromosomal translocations represent an important class of these abnormalities and often lead to the formation of oncogenic fusion proteins that involve transcription factors or transcriptional regulators. One of the best characterized translocations results in the fusion of the EWSR1 gene and the FLI1 ETS transcription factor in Ewing sarcoma, the second most common pediatric bone cancer (Delattre et al., 1992). The EWS-FLI1 oncogenic fusion protein is often the only genetic alteration in these tumors (Brohl et al., 2014; Crompton et al., 2014; Tirode et al., 2014) and operates as an aberrant transcription factor containing the ETS DNA-binding domain of FLI1. EWSR1 has been linked to transcriptional activation and RNA binding (Kovar, 2011), yet its contribution to the function of EWS-FLI1 remains poorly defined. Accordingly, there remains a great need in the art to identify therapeutic agents and methods that target EWS-FLI1 to treat Ewing sarcoma.

### SUMMARY OF THE INVENTION

The present invention is based, at least in part, on the discovery that the BAF complex is recruited by the EWS-FLI1 fusion protein to tumor-specific enhancers and contributes to target gene activation in Ewing sarcoma. This process is a neomorphic property of EWS-FLI1 compared to wildtype FLI1 and depends on tyrosine residues that are necessary for phase transitions of the EWSR1 prion-like domain. Furthermore, fusion of short fragments of EWSR1 to FLI1 is sufficient to recapitulate BAF complex retargeting and EWS-FLI1 activities.

In one aspect, a method of treating a subject afflicted with cancer comprising administering to the subject a therapeutically effective amount of an agent that inhibits binding of a FET-ETS fusion protein to a BAF complex, is provided. In another aspect, a method of treating a subject afflicted with cancer comprising administering to the subject a therapeutically effective amount of an agent that inhibits binding of a BAF complex to at least one FET-ETS fusion protein-bound GGAA repeat enhancer, is provided. In still another aspect, a method of reducing viability or proliferation of cancer cells comprising contacting the cancer cells with an agent that inhibits binding of a FET-ETS fusion protein to a BAF complex, is provided. In yet another aspect, a method of reducing viability or proliferation of cancer cells comprising contacting the cancer cells with an agent that inhibits binding of a BAF complex to at least one FET-ETS fusion protein-bound GGAA repeat enhancer is provided.

Numerous embodiments are further provided that can be applied to any aspect of the present invention and/or combined with any other embodiment described herein. For example, in one embodiment, the agent is a small molecule inhibitor, a small molecule degrader, CRISPR guide RNA (gRNA), RNA interfering agent, oligonucleotide, peptide or peptidomimetic inhibitor, aptamer, antibody, or intrabody. The RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA). In another embodiment, the agent comprises an antibody and/or intrabody, or an antigen binding fragment thereof, which specifically binds to the FET-ETS fusion protein or the BAF complex. In still another embodiment, the antibody and/or intrabody, or antigen binding fragment thereof, is chimeric, humanized, composite, or human. In yet another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

In one embodiment, the method further comprises administering to the subject at least one additional therapeutic agent or regimen for treating the cancer. In certain embodiments, the method further comprises administering an immunotherapy and/or cancer therapy, optionally wherein the immunotherapy and/or cancer therapy is administered before, after, or concurrently with the agent. In one embodiment, the immunotherapy is cell-based. In another embodiment, the immunotherapy comprises a cancer vaccine and/or virus. In still another embodiment, the immunotherapy inhibits an immune checkpoint. For example, the immune checkpoint may be any checkpoint protein known in the art, such as one selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the cancer therapy is selected from the group consisting of radiation, a radiosensitizer, and a chemotherapy.

In another embodiment, the agent reduces the number of viable or proliferating cells in the cancer, and/or reduces the volume or size of a tumor comprising the cancer cells. In still another embodiment, the agent inhibits the binding of the FET-ETS fusion protein to the BAF complex, and decreases binding of the BAF complex to at least one FET-ETS fusion protein-bound GGAA repeat enhancer. In some embodiments, the FET-ETS fusion protein-bound GGAA repeat enhancer is associated with a gene selected from the group consisting of KIT, CCND1, NKX2-2, SOX2, NR0B1, EZH2, and LINC00221. In yet another embodiment, the agent decreases expression of at least one target gene of the FET-ETS fusion protein. In a further embodiment, the target gene of the FET-ETS fusion protein is selected from the group consisting of NKX2-2, NPY1R, PPP1R1A, KIT, LOXHD1, MAFB, and NGFR.

In another aspect, a method of assessing the efficacy of an agent for treating cancer in a subject is provided, the method comprising: a) detecting in a subject sample at a first point in time the amount of at least one gene selected from the group consisting of NKX2-2, NPY1R, PPP1R1A, KIT, LOXHD1, MAFB, and NGFR; b) repeating step a) during at least one subsequent point in time after administration of the agent; and c) comparing the amount detected in steps a) and b), wherein the absence of, or a significant decrease in amount of at least one gene selected from the group consisting of NKX2-2, NPY1R, PPP1R1A, KIT, LOXHD1, MAFB, and NGFR in the subsequent sample as compared to the amount in the sample at the first point in time, indicates that the agent treats cancer in the subject. In one embodiment, the first and/or at least one subsequent sample is selected from the group consisting of ex vivo and in vivo samples. In another embodiment, the first and/or at least one subsequent sample is obtained from an animal model of the cancer. In still another embodiment, the first and/or at least one subsequent sample is a portion of a single sample or pooled samples obtained from the subject. The sample may comprise cells, serum, peritumoral tissue, and/or intratumoral tissue obtained from the subject. In yet another embodiment, the method further comprises determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

In still another aspect, a cell-based assay for screening for agents that reduce viability or proliferation of a cancer cell is provided, wherein the assay comprises contacting the cancer cell with a test agent, and determining the ability of the test agent to decrease (1) binding of a FET-ETS fusion protein to a BAF complex; (2) binding of a BAF complex to at least one FET-ETS fusion protein-bound GGAA repeat enhancer; and/or (3) expression of at least one target gene of the FET-ETS fusion protein. In one embodiment, the step of contacting occurs in vivo, ex vivo, or in vitro. In another embodiment, the FET-ETS fusion protein-bound GGAA repeat enhancer is associated with a gene selected from the group consisting of KIT, CCND1, NKX2-2, SOX2, NR0B1, EZH2, and LINC00221. In still another embodiment, the target gene of the FET-ETS fusion protein is selected from the group consisting of NKX2-2, NPY1R, PPP1R1A, KIT, LOXHD1, MAFB, and NGFR. In yet another embodiment, the cell-based assay further comprises determining a reduction in the viability or proliferation of the cancer cells.

In yet another aspect, an in vitro assay for screening for agents that reduce viability or proliferation of cancer cells is provided, comprising: a) mixing a FET-ETS fusion protein-bound GGAA repeat enhancer, a FET-ETS fusion protein, and a BAF complex together; b) adding a test agent to the mixture; and c) determining the ability of the test agent to decrease binding of the FET-ETS fusion protein to the BAF complex, and/or binding of the BAF complex to the FET-ETS fusion protein-bound GGAA repeat enhancer. In one embodiment, the FET-ETS fusion protein-bound GGAA repeat enhancer is associated with a gene selected from the group consisting of KIT, CCND1, NKX2-2, SOX2, NR0B1, EZH2, and LINC00221.

As described herein, numerous embodiments are further provided that can be applied to any aspect of the present invention and/or combined with any other embodiment described herein. For example, in one embodiment, the BAF complex is a human BAF complex. In another embodiment, the FET-ETS fusion protein binds to at least one subunit of the BAF complex, wherein the subunit is selected from the group consisting of SMARCA2, SMARCA4/BRG1, SMARCB1/BAF47, SMARCC1/BAF155, SMARCC2/BAF170, SMARCD1/BAF60A, SMARCD2, SMARCE1/BAF157, DPF2/RAF45D, ARID1A/BAF250A, ARID1B/BAF250B, SS18, and ACTL6A/BAF53A. In still another embodiment, the FET-ETS fusion protein consists of an N-terminal portion of a FET protein and a C-terminal portion of an ETS protein. In yet another embodiment, the binding of the FET-ETS fusion protein to the BAF complex is dependent on a prion-like domain of the N-terminal portion of the FET protein. In certain embodiments, the binding of the FET-ETS fusion protein to the BAF complex is dependent on the tyrosine residues in the prion-like domain. In one embodiment, the FET protein is selected from the group consisting of FUS, TAF15, and EWSR1. In another embodiment, the ETS protein is selected from the group consisting of FLI1, ERG, ETV1, and ETS1. In still another embodiment, the cancer is selected from the group consisting of leukemia, Ewing sacoma and primitive neuroectodermal tumor (PNET). In certain embodiment, the FET-ETS fusion protein is EWS-FLI1, and the cancer is Ewing sacoma or primitive neuroectodermal tumor (PNET). In one embodiment, the subject is an animal model of the cancer. In another embodiment, the subject is a mammal, such as a human. In still another embodiment, the agent is a RAF complex inhibitor. The BAF complex inhibitor described in the instant disclosure may be any BAF complex inhibitor known in the art, such as one selected from the group consisting of Bromosporine, LP99, I-BRD9, BI-9564, BI-7273, GSK-39, dBRD9, and PFI-3. In yet another embodiment, the agent is administered in a pharmaceutically acceptable formulation.

### BRIEF DESCRIPTION OF THE DRAWINGS

FIG. 1. EWS-FLI1 binds mSWI/SNF (BAF) complexes and co-localizes at tumorspecific GGAA repeat enhancer elements in Ewing sarcoma.

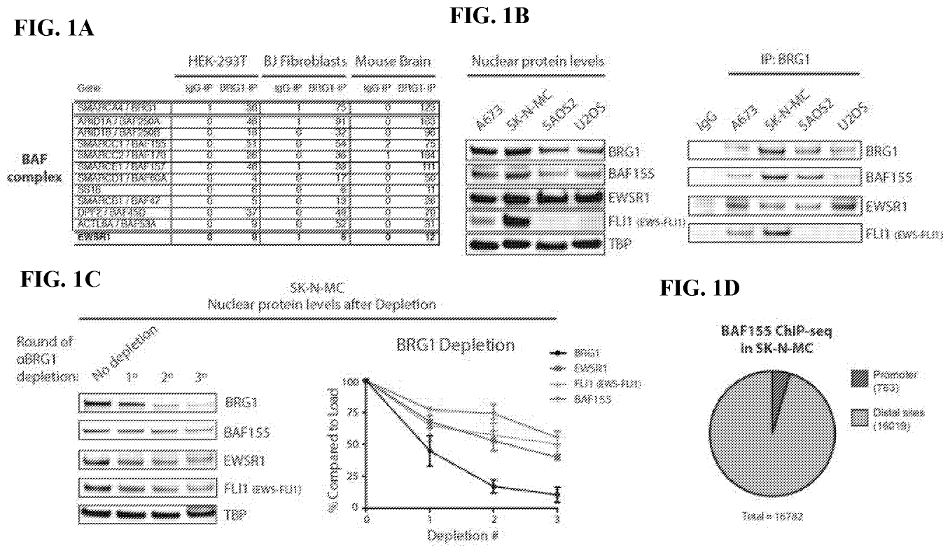

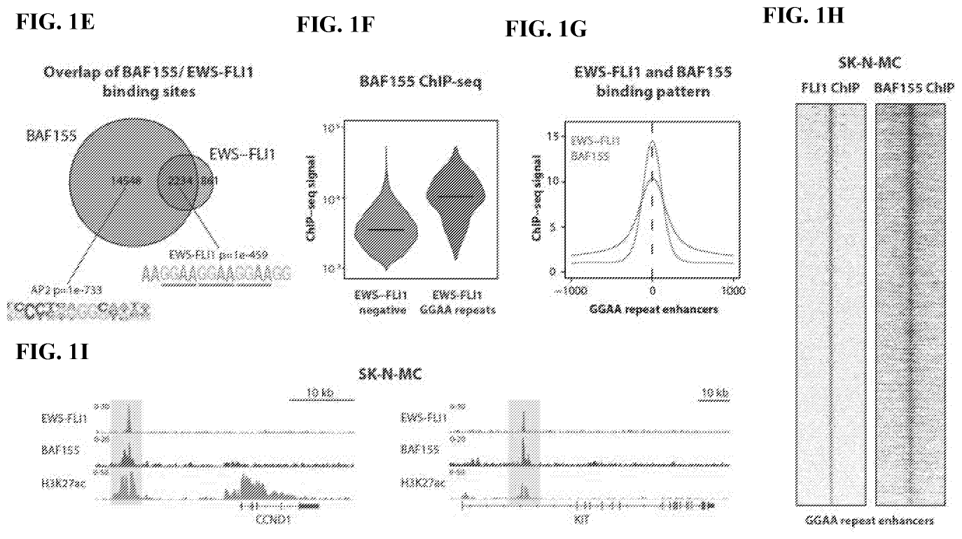

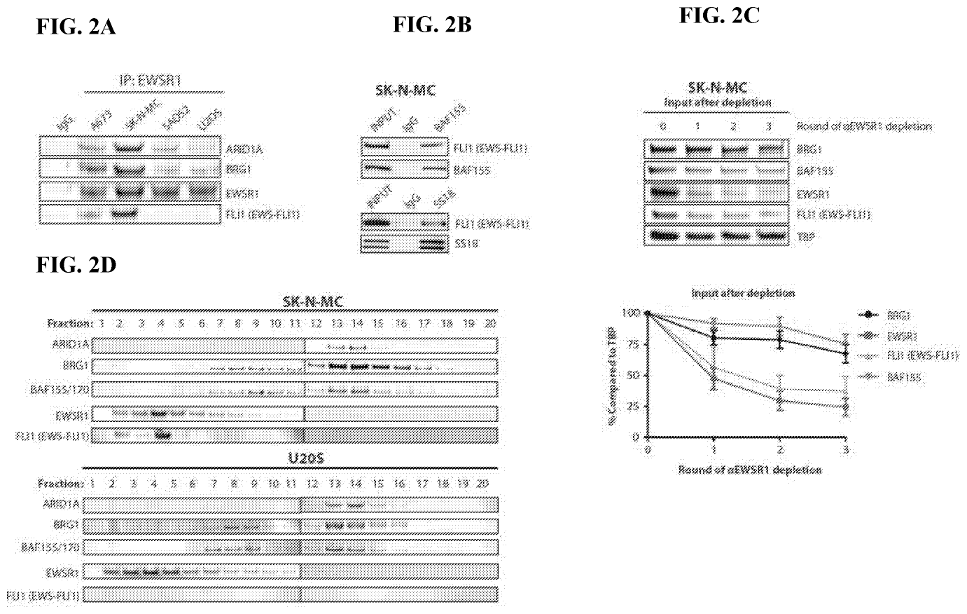

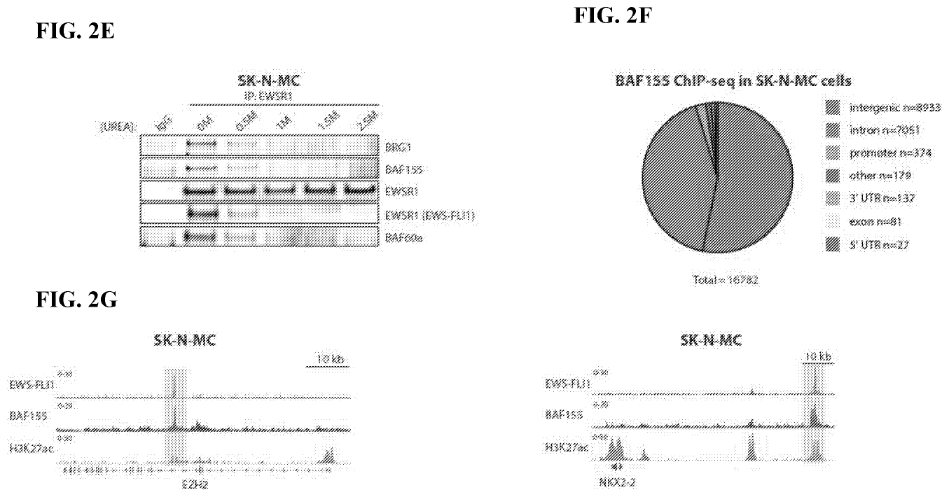

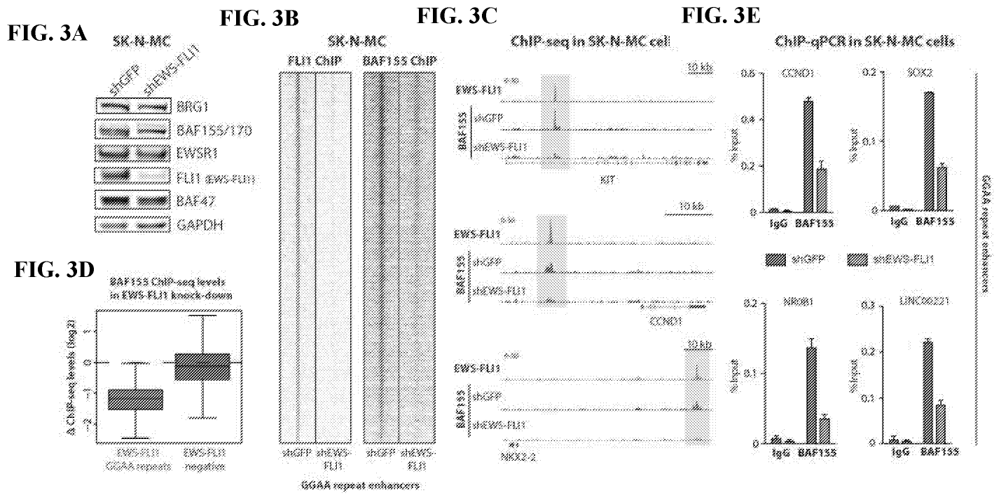

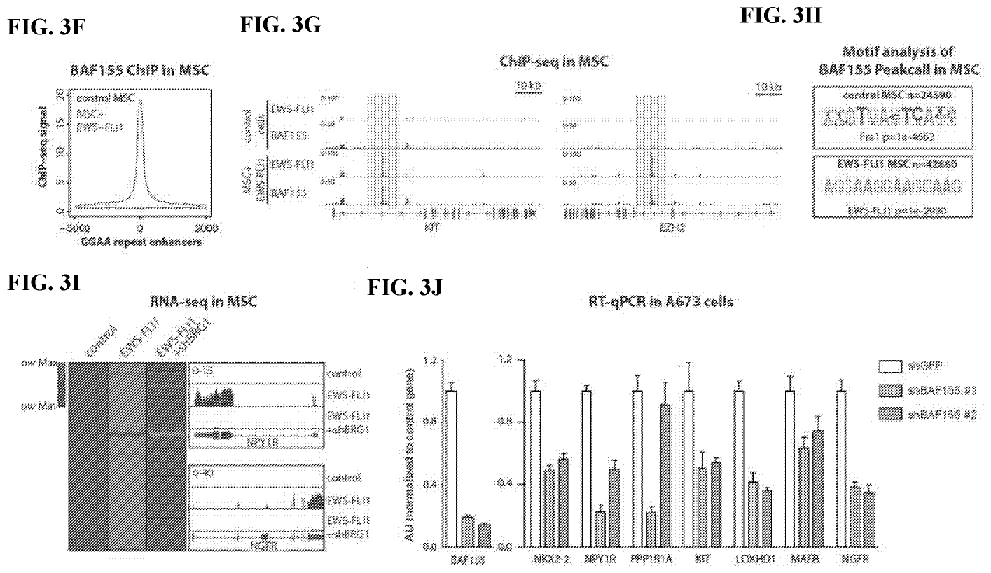

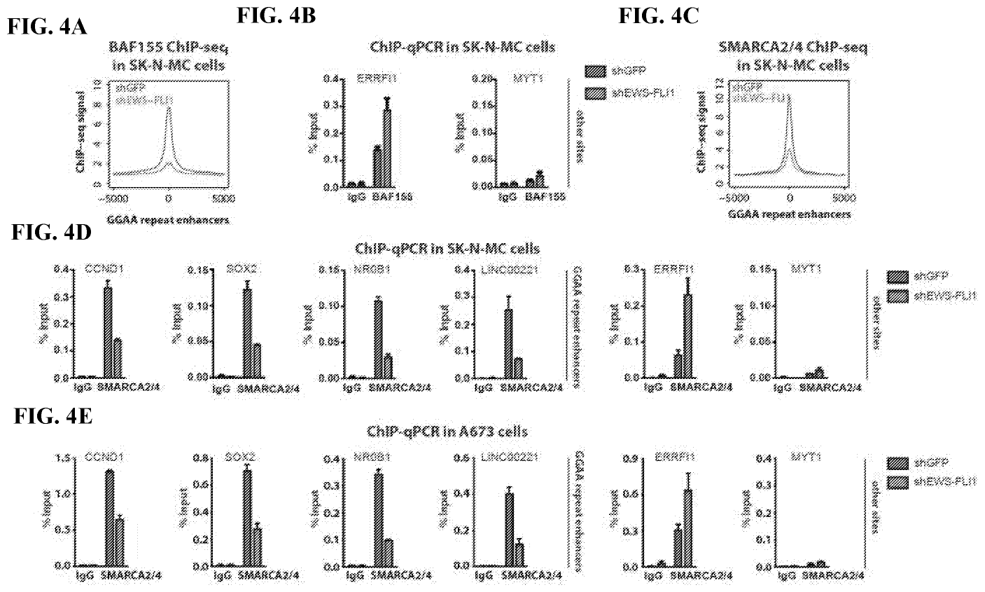

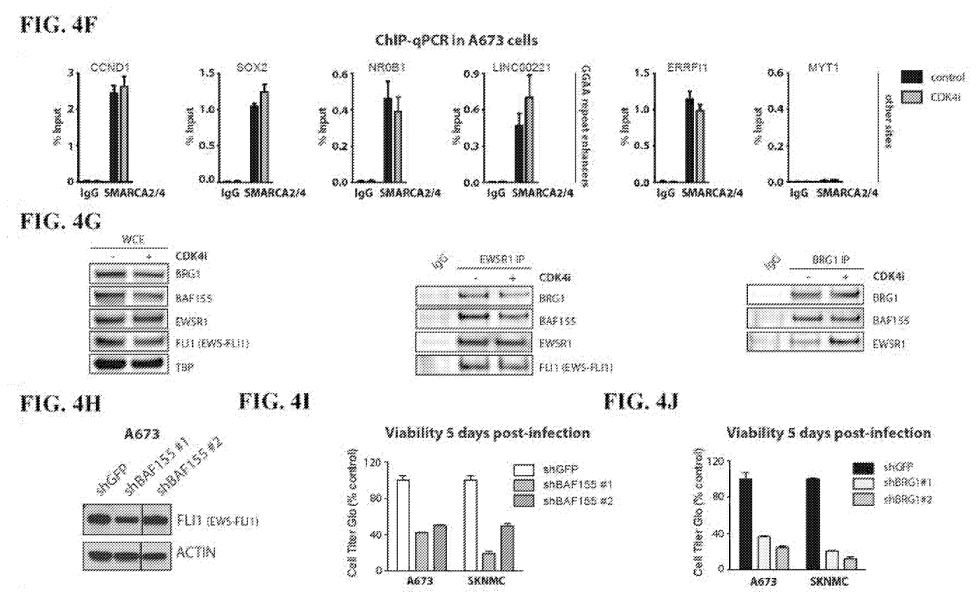

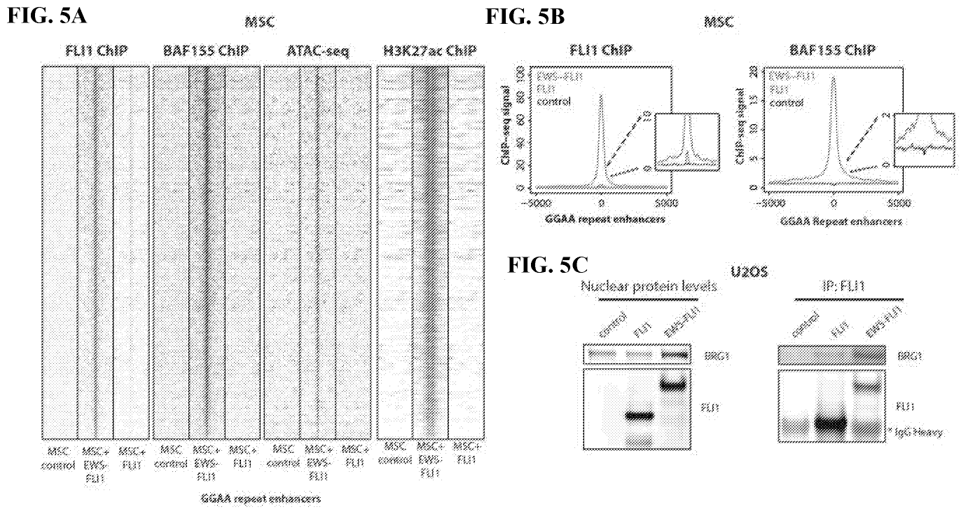

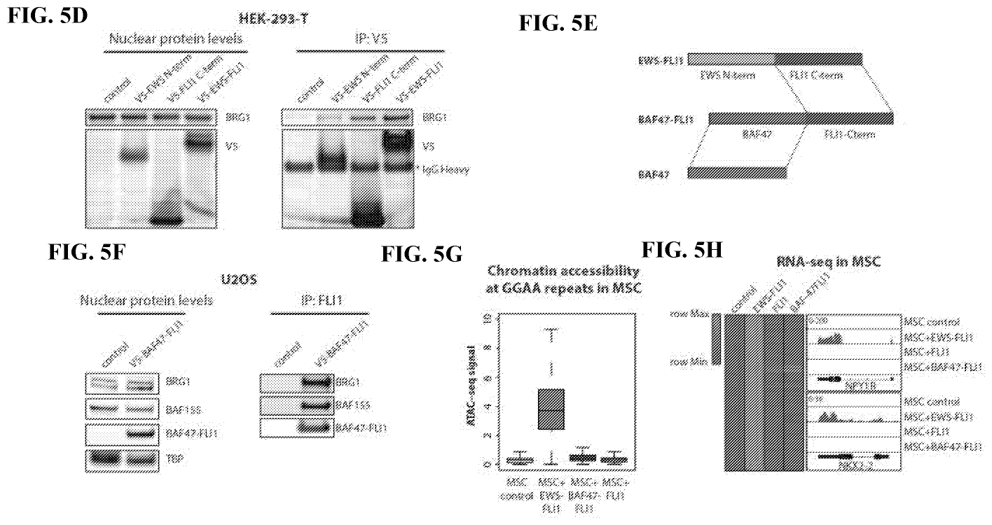

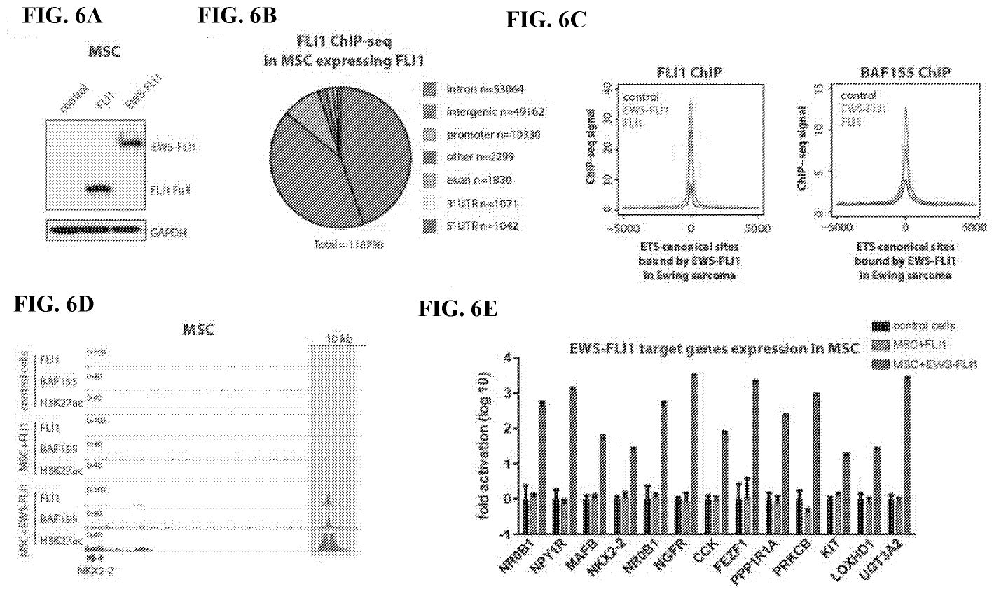

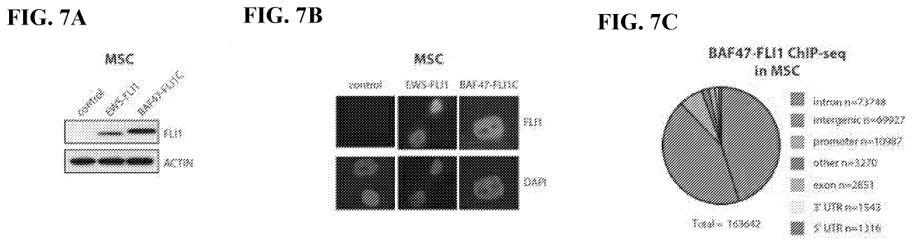

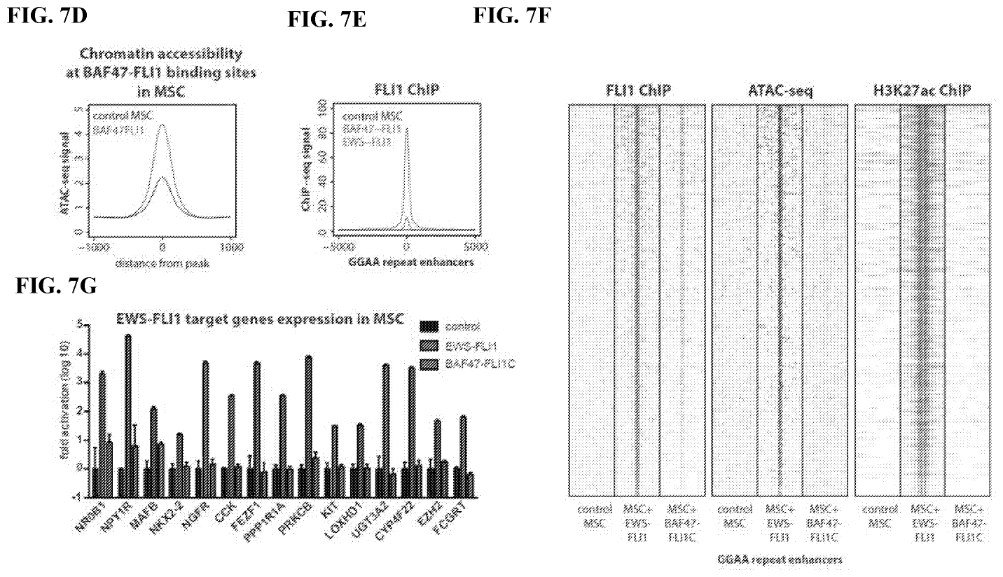

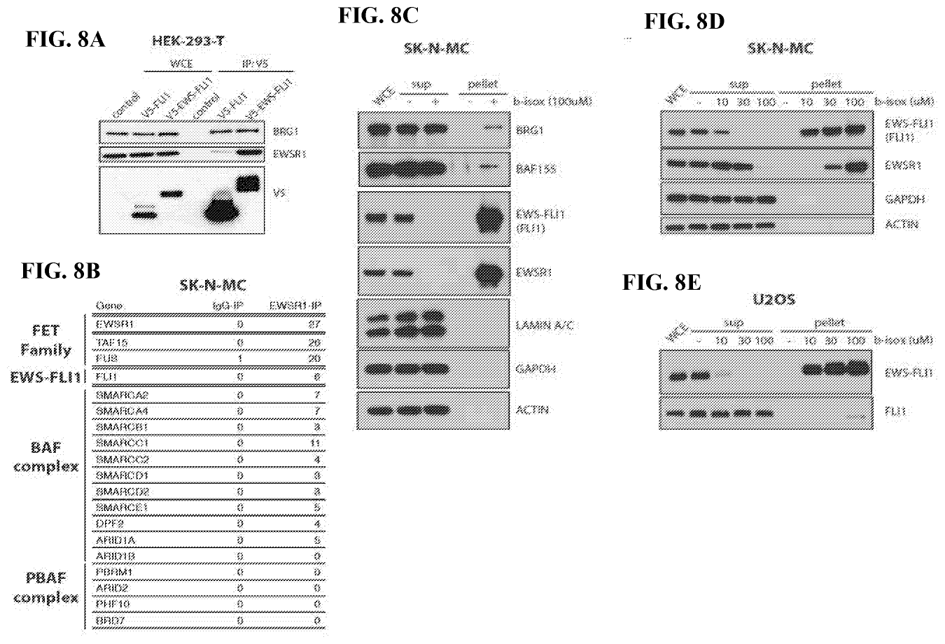

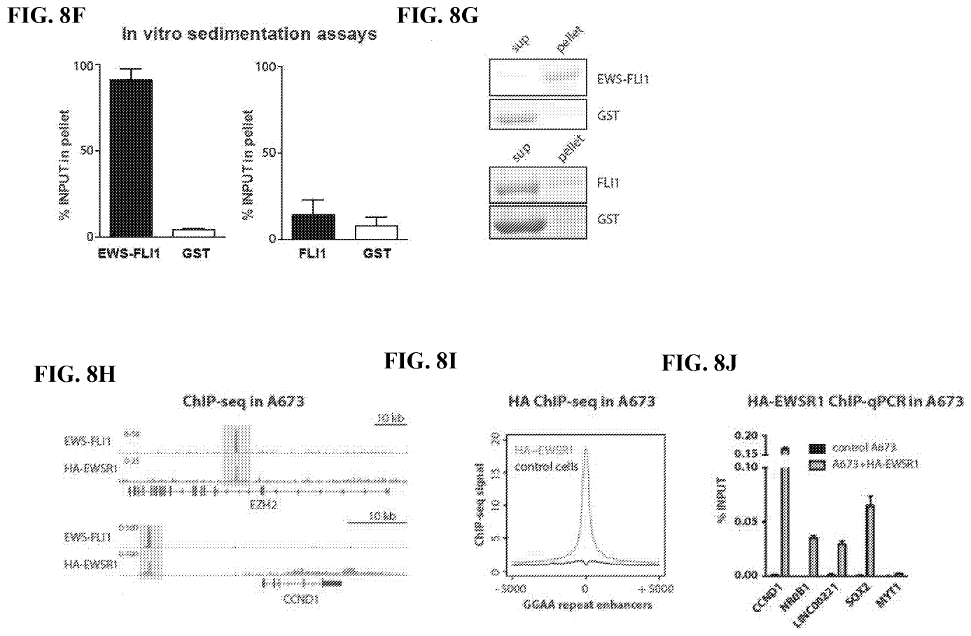

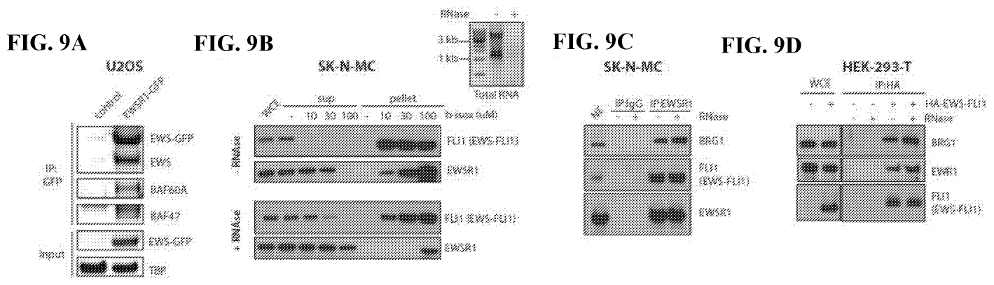

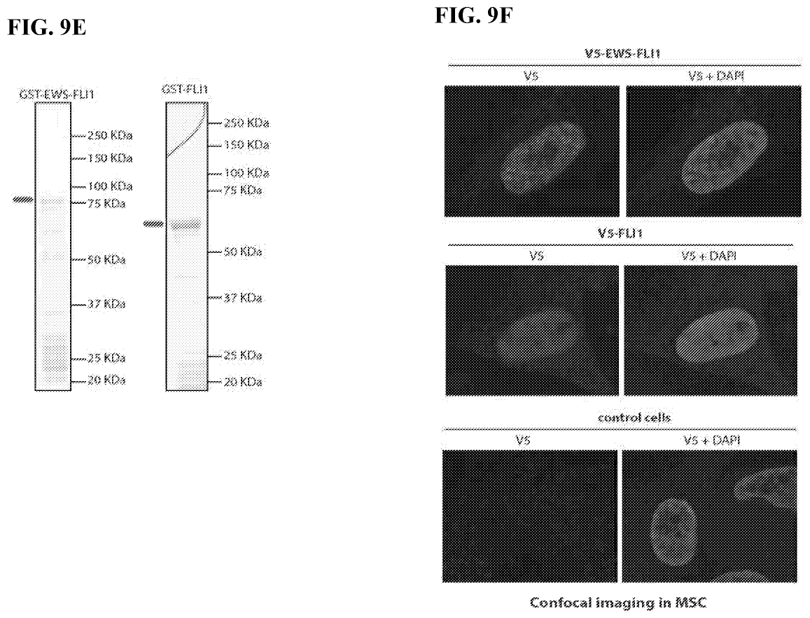

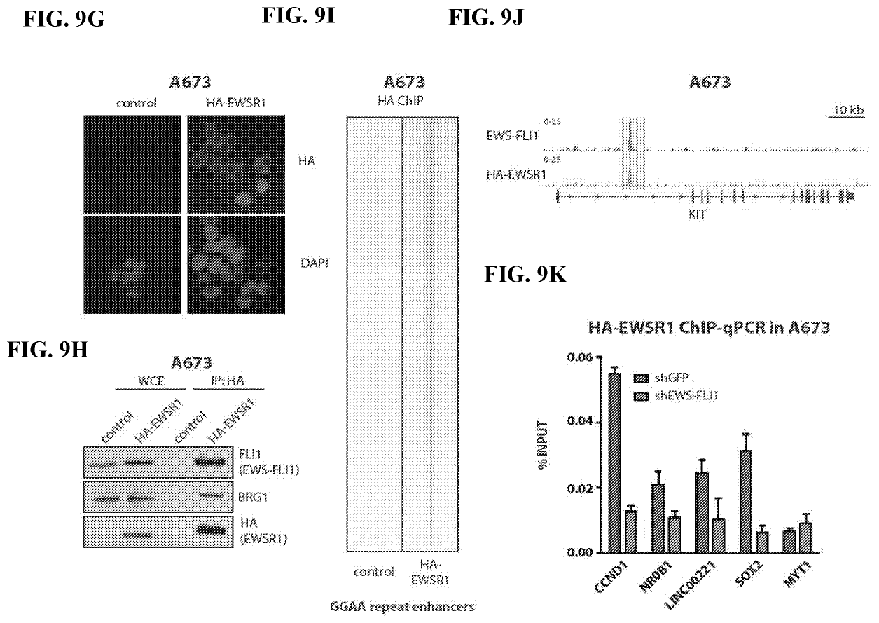

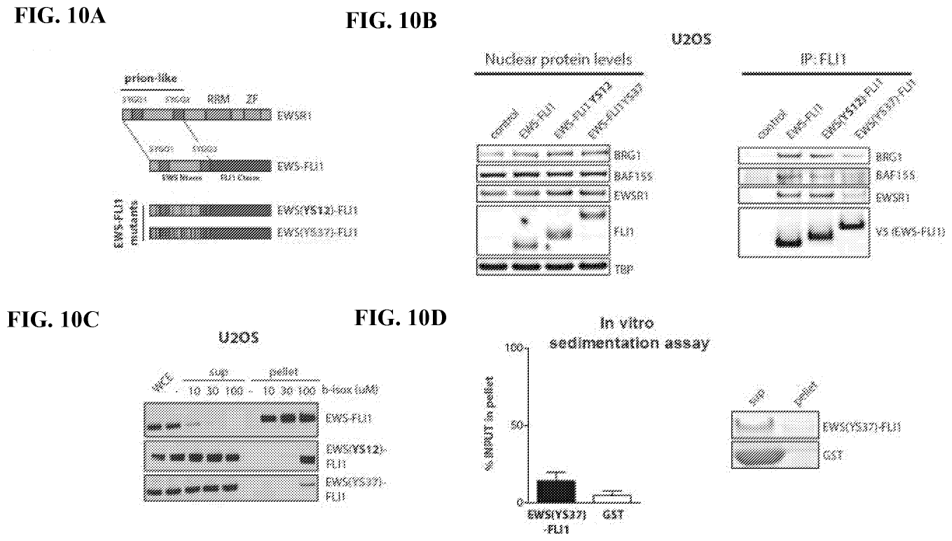

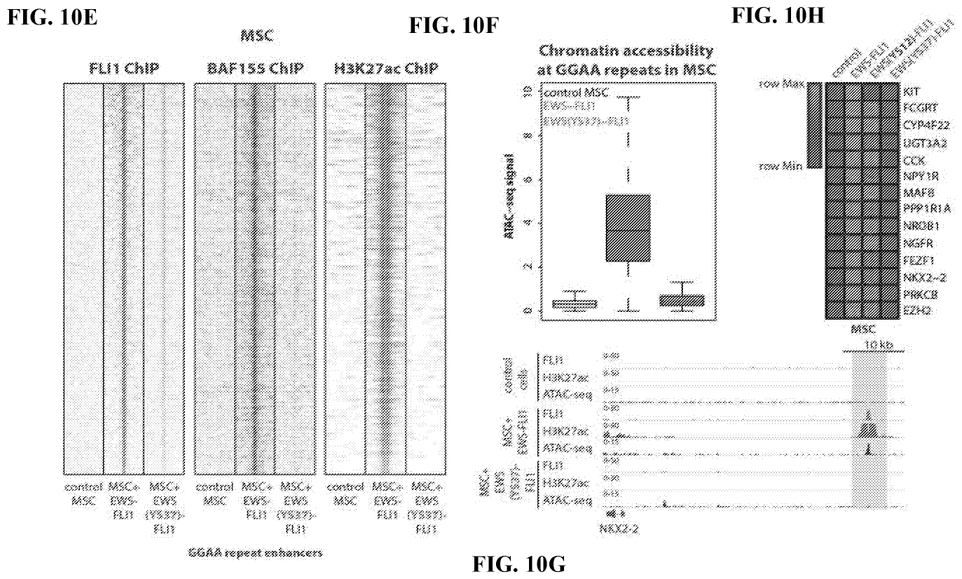

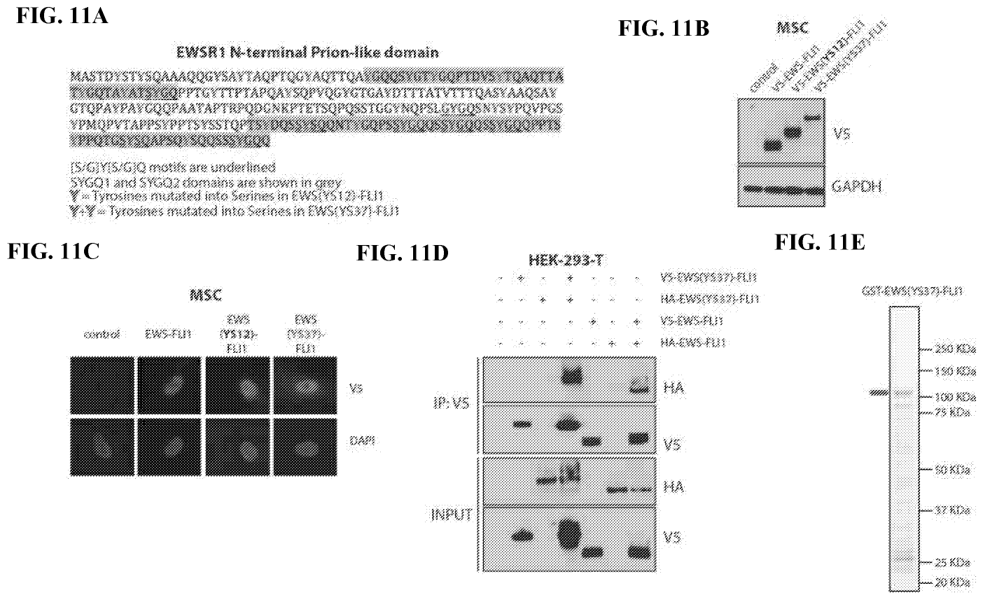

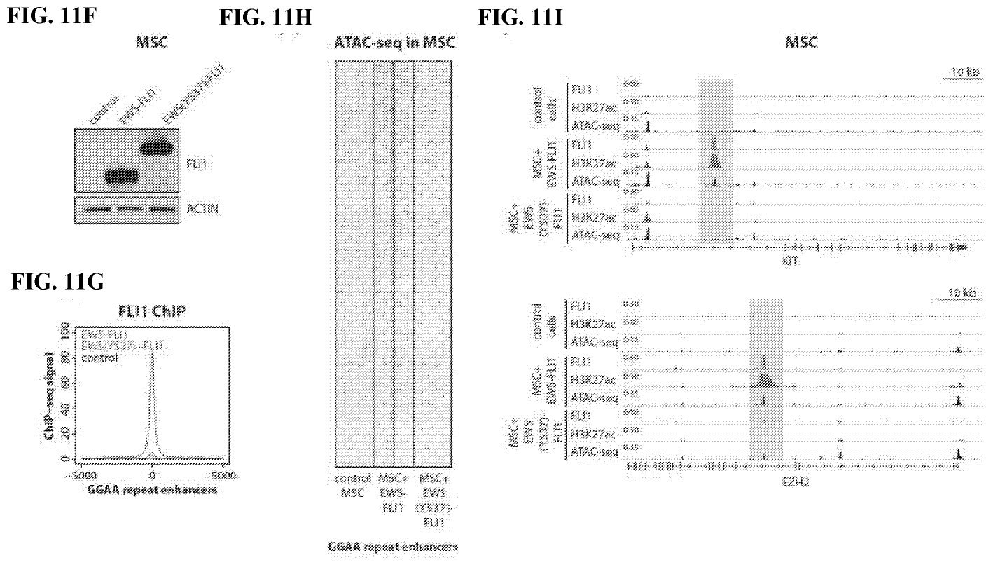

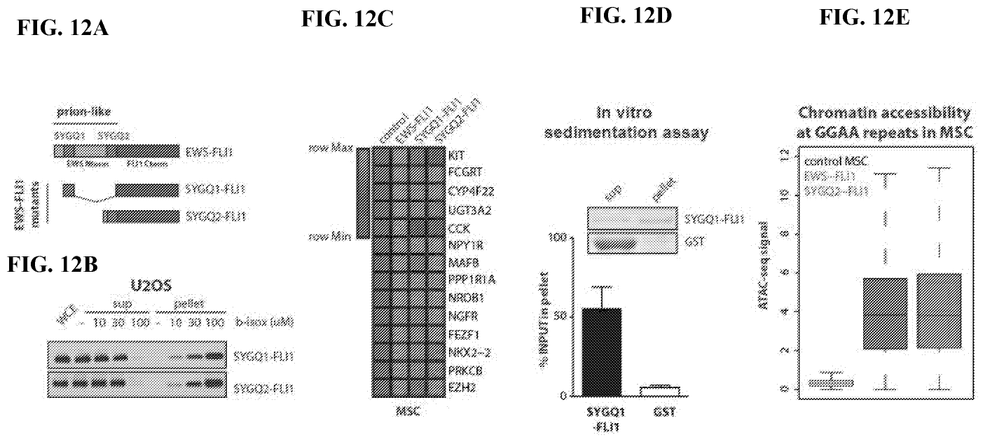

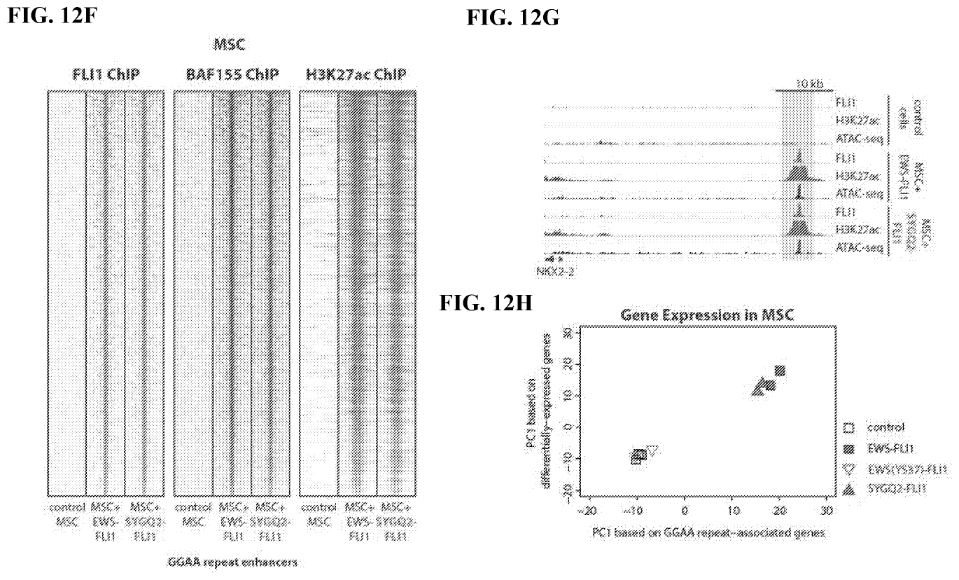

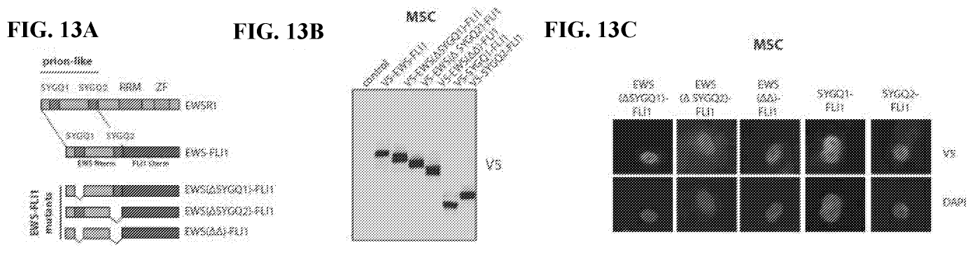

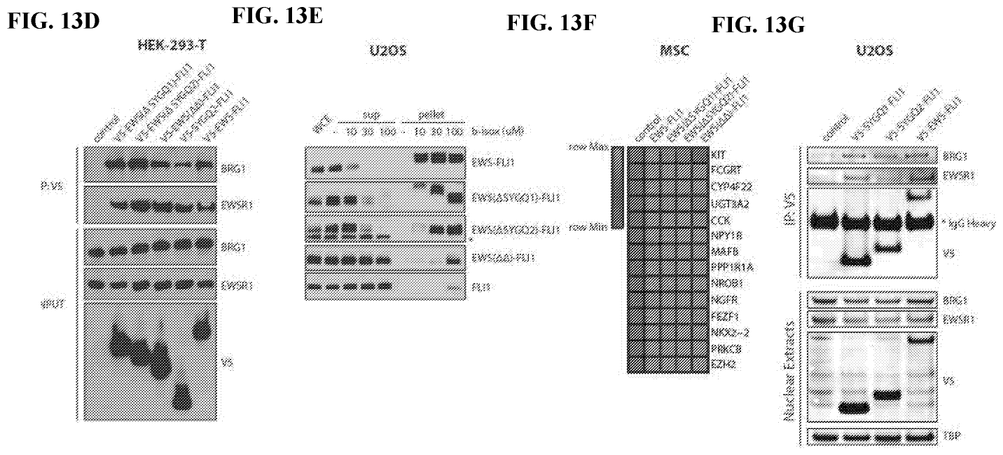

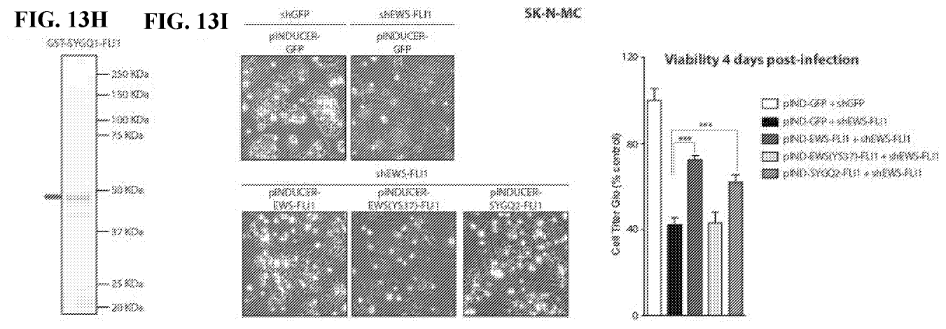

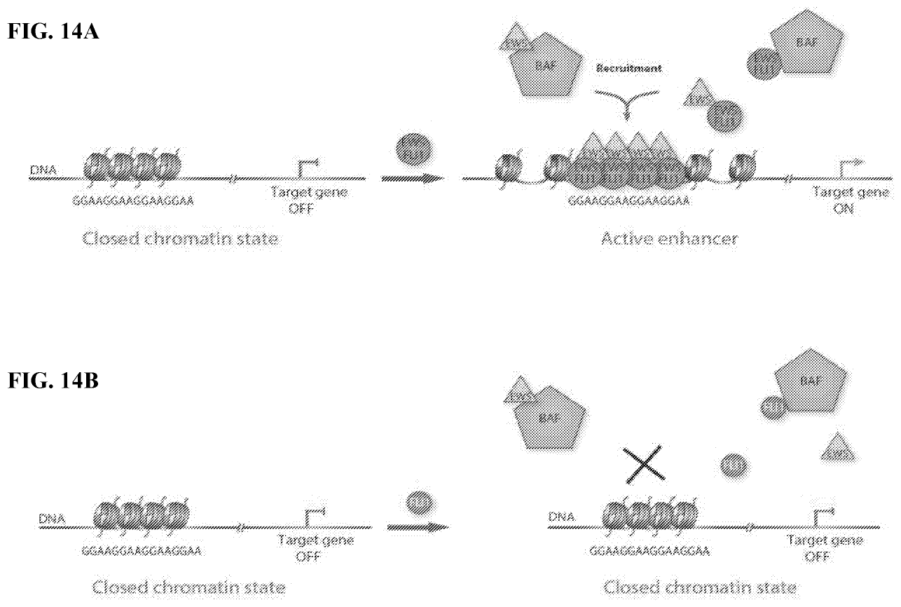

(FIG. 1A) Table highlighting enrichment of EWSR1 peptides in anti-BRG1 immunoprecipitation/mass spectrometry studies in human cells (HEK-293-T and BJ fibroblasts) and in mouse brain tissue. Highlighted are the number of peptides of EWSR1 and BAF complex members.

(FIG. 1B) (Left) Immunoblotting for BAF subunits, EWSR1 and EWS-FLI1 performed on nuclear extracts used for immunoprecipitation. (Right) Immunoprecipitation studies using an anti-BRG1 antibody in Ewing sarcoma (A673, SK-N-MC), and osteosarcoma (SAOS2, U2OS) cell lines demonstrate binding of EWSR1 (wild-type) and EWS-FLI1 to BAF complexes.

(FIG. 1C) (Left) Tmmunodepletion studies performed on SK-N-MC Ewing sarcoma nuclear extracts using an anti-BRG1 antibody. (Right) Quantification of depletion experiments using quantitative densitometry. Error bars represent SEM of n=2 independent experiments.

(FIG. 1D) Distribution of MACS-called BAF155 ChIP-seq peaks in SK-N-MC Ewing sarcoma cells. BAF complexes (as marked by BAF155) are primarily localized at putative enhancer sites. Promoters are annotated using the Refseq promoter database.

(FIG. 1E) Venn diagram depicting the overlap of BAF155 and EWS-FL1 (FLI1) MACS-called peaks in Ewing sarcoma SK-N-MC cells. The top motifs for distal sites with BAF155-only or BAF155/EWS-FLI1 overlap are shown. The GGAA motif recognized by EWS-FLI1 is underlined in red. Motifs were identified using HOMER.

(FIG. 1F) Total BAF155 ChIP-seq signals at BAF-155-only sites (N=14548) and sites cohound with EWS-FLI1 at GGAA repeats (N=660) as represented by violin plots.

(FIG. 1G) Composite plot shows EWS-FLI1 and BAF155 ChIP-seq signals at overlapping GGAA repeat binding sites. The x-axis represents a 2 kb window centered on EWS-FLI1 binding sites.

(FIG. 1H) Heatmaps showing EWS-FLI1 and BAF155 ChIP-seq signal density in Ewing sarcoma SK-N-MC cells. 10 kb windows in each panel are centered on EWS-FLI1-bound GGAA repeat enhancer sites (N=812).

(FIG. 1I) Representative examples of EWS-FLI1 and BAF155 co-occupancy shown at GGAA repeat enhancers associated with the CCND1 and KIT genes. Enhancer regions are highlighted in light gray.

FIG. 2. EWSR1 wild type and the fusion protein EWS-FLI1 interact with mSWI/SNF (BAF) complexes but are not subunits.

(FIG. 2A) Immunoprecipitations using an anti-EWSR1 N-terminal antibody in Ewing sarcoma (A673 and SK-N-MC) and osteosarcoma (SAOS2 and U2OS) nuclear extracts confirm EWSR1-BAF and EWS-FLI1-BAF interactions.

(FIG. 2B) Co-immunoprecipitation experiments using antibodies specific to BAF155 or SS18 in SK-N-MC nuclear extracts show that EWS-FLI1 interacts with BAF complexes.

(FIG. 2C) (Top) Immunodepletion studies performed on SK-N-MC nuclear extracts using an anti-EWSR1 N-terminal antibody. (Bottom) Quantification of depletion experiments (average and SEM of 3 independent experiments).

(FIG. 2D) Density sedimentation studies using 10-30% glycerol gradient analyses of nuclear extracts from SK-N-MC and U2OS cells; immunoblot for EWSR1, EWS-FLI1 and BAF complex subunits.

(FIG. 2E) Urea denaturation analysis performed on anti-EWSR1 C-terminal IPs from SK-NMC nuclear extracts treated with [urea]=0-2.5M. EWS-FLI1 and the BAF complex are held with equimolar affinity to EWSR1.

(FIG. 2F) Pie chart showing annotations of MACS-called peaks for BAF155 ChIP-seq in SKNMC cells using Homer.

(FIG. 2G) Additional representative examples of EWS-FLI1 and BAF155 co-occupancy at GGAA repeat enhancers associated with EZH2 and NKX2-2 genes in SK-N-MC cells.

FIG. 3. Interdependency of EWS-FLI1 and BAF complexes in driving oncogenic gene expression programs in Ewing sarcoma.

(FIG. 3A) shRNA-mediated suppression of EWS-FLI1 (versus shGFP as control) in SK-N-MC Ewing sarcoma cells; immunoblot for Fill (EWS-FLI1), EWSR1 and BAF complex subunits performed on nuclear extracts.

(FIG. 3B) Heatmaps showing EWS-FLI1 and BAF155 ChIP-seq signal density in SK-N-MC cells treated with either shGFP control or shEWS-FLI1 knockdown. 10 kb windows in each panel are centered on EWS-FLI1-bound GGAA repeat enhancer sites (N=812).

(FIG. 3C) Example tracks demonstrating decreased binding of BAF155 at EWS-FLI1 bound GGAA repeat enhancers associated with KIT, CCND1 and NKX2-2 in SK-N-MC cells treated with either shGFP or shEWS-FLI1 knockdown. Enhancer regions of interest are highlighted in light gray.

(FIG. 3D) BAF155 occupancy is decreased specifically at GGAA repeat regions following EWS-FLI1 knock down in SK-N-MC cells. Box plots depict the changes in BAF155 ChIPseq signals between SK-N-MC cells treated with either shGFP or shEWS-FLI1 knockdown. BAF155 MACS-called peaks are divided into EWS-FLI1 bound GGAA repeat enhancers (n=660 sites, purple) and BAF155-only sites (n=14538 peaks, blue).

(FIG. 3E) ChIP-qPCR validation of decreased BAF155 occupancy at selected EWS-FLI1 GGAA repeat enhancers associated with CCND1, SOX2, NR0B1, and LINC00221.

(FIG. 3F) Introduction of EWS-FLI1 in MSCs results in recruitment of BAF complexes to GGAA microsatellite repeats. Composite plot shows BAF155 ChIP-seq signals in control MSCs (black) and EWS-FLI1-expressing MSCs (blue). The x-axis represents a 10 kb window centered on EWS-FLI1 binding sites.

(FIG. 3G) Examples of recruitment of BAF155 by EWS-FLI1. ChIP-seq tracks illustrate EWSFLI1 and BAF155 binding at GGAA repeat microsatellites upon introduction of EWSFLI1 into MSCs. Enhancer regions of interest are highlighted in light gray.

(FIG. 3H) HOMER motif analysis on BAF155 MACS-called peaks in control conditions and on newly created peaks after EWS-FLI1 expression in MSCs indicates that EWS-FLI1 redistributes BAF155 to Ewing sarcoma GGAA repeat enhancer elements.

(FIG. 3I) BAF complex activity is required for activation of EWS-FLI1 target genes. (Left) Heatmap shows relative RNA-seq gene expression levels of genes associated with GGAA repeats and activated upon introduction of EWS-FLI1 in MSCs (rows, N=79 genes). The columns show MSCs treated with either empty vector control, EWS-FLI1+shGFP, or EWS-FLI1+shBRG1. Expression values were normalized by row. (Right) Example RNA-seq tracks over selected genes.

(FIG. 3J) RT-qPCR experiments show decreased mRNA expression of EWS-FLI1 target genes 48 hours post-infection with BAF155 shRNA in A673 Ewing sarcoma cells.

FIG. 4. Loss of EWS-FLI1 results in a specific decreased occupancy of BAF complexes over GGAA repeats enhancer elements in Ewing Sarcoma.

(FIG. 4A) Composite plot shows that BAF155 occupancy is strongly reduced at EWS-FLI1 bound GGAA repeat enhancer sites upon EWS-FLI1 knock-down in SK-N-MC cells.

(FIG. 4B) ChIP-qPCR experiments at control sites confirm the specificity of BAF155 decreased occupancy observed at GGAA repeats in FIG. 2E upon EWS-FLI1 knockdown in SKNMC cells. ERRFI1 is an ETS binding site repressed by EWS-FLI1 and MYT1 is a negative control region.

(FIG. 4C) Composite plot shows that SMARCA2/4 occupancy is strongly reduced at EWSFLI1 bound GGAA repeat enhancer sites upon EWS-FLI1 knock-down in SKNMC cells.

(FIG. 4D-E) ChIP-qPCR experiments show specific decreased occupancy of SMARCA2/4 upon EWS-FLI1 knock-down in SKNMC (FIG. 4D) and A673 cells (FIG. 4E) at GGAA repeats. CCND1, SOX2, NR0B1 and LINC00221 are GGAA repeat microsatellites activated by EWS-FLI1 in Ewing sarcoma. ERRFI1 is an ETS binding site repressed by EWS-FLI1 and MYT1 is a negative control region.

(FIG. 4F) ChIP-qPCR experiments show maintained occupancy of SMARCA2/4 at GGAA repeat enhancers upon CDK4i (palbociclib) treatment in A673 cells.

(FIG. 4G) Co-immunoprecipitation experiments show maintained interactions between EWSR1, EWS-FLI1 and BAF complex upon CDK4i (palbociclib) treatment in A673 cells.

(FIG. 4H) Immunoblotting shows maintained EWS-FLI1 protein levels in A673 cells infected with two different shRNAs targeting BAF155 as shown FIG. 2J.

(FIG. 4I-J) Cell viability assays (Cell-titer Glo) in A673 and SKNMC cells five days postinfection with two different shRNAs specifically targeting BAF155 (FIG. 4I) and BRG1 (FIG. 4J).

FIG. 5. Recruitment of BAF complexes to GGAA microsatellite repeats is a neomorphic property of EWS-FLI1.

(FIG. 5A) Heatmaps of FLI1, BAF155, H3K27ac ChIP-seq and ATAC-seq signal densities in MSCs infected with either control vector, EWS-FLI1 or wild-type FLI1. 10 kb windows in each panel are centered on EWS-FLI1-bound GGAA repeat enhancer sites (N=812).

(FIG. 5B) Composite plots show FLI1 (left) and BAF155 (right) ChIP-seq occupancy over GGAA repeat enhancers in control MSCs and MSCs expressing EWS-FLI1 or FLI1. The x-axis represents a 10 kb window centered on EWS-FLI1 binding sites. Inset: 10 fold magnification showing minimal wild-type FLI1 binding over repeat enhancers but no BAF155 recruitment by FLI1.

(FIG. 5C) Both wild-type FLI1 and EWS-FLI1 interact with BAF complexes. (Left) Immunoblots from nuclear extracts show lentiviral expression of wild-type FLI1 or EWSFLI1 and the levels of endogenous BRG1 in U2OS cells. (Right) Co-immunoprecipitation experiments using anti-Fill antibodies show interactions with BAF. * Indicates IgG Heavy chains used for immunoprecipitation; they migrate at a size similar to wild-type FLI1.

(FIG. 5D) EWS-FLI1 interacts with BAF complexes through both EWS N-terminal and FLI1 Cterminal fragments. (Left) Immunoblots from nuclear extracts show the expression of transiently transfected V5-EWSR1 N-terminal, V5-FLI1 C-terminal, or V5-EWS-FLI1 and the levels of endogenous BRG1 in HEK-293-T cells. (Right) Co-immunoprecipitation experiments using anti-V5 antibodies show interactions with BAF. * Indicates IgG Heavy chains used for immunoprecipitation.

(FIG. 5E) Schematic of the BAF47-FLI1 fusion protein used in experiments in relation to EWS-FLI1 and BAF47.

(FIG. 5F) (Left) Immunoblots from nuclear extracts show lentiviral expression of BAF47-FLI1 fusion protein and the levels of endogenous BAF proteins in U2OS cells. (Right) Anti-FLI1 immunoprecipitation confirms an interaction between BAF47-FLI1 and BAF complex subunits.

(FIG. 5G) ATAC-seq signal intensity indicative of chromatin accessibility at GGAA repeat microsatellites in MSCs infected with either control vector, EWS-FLI1, BAF47-FLI1 or FLI1 wild-type. Only EWS-FLI1 induces increased accessibility at GGAA repeat enhancers.

(FIG. 5H) The fusion of the FLI1 C-terminal region to BAF47 is not sufficient for the activation of EWS-FLI1 target genes. Heatmap shows relative RNA-seq gene expression levels of genes associated with GGAA repeats and activated upon introduction of EWS-FLI1 in MSCs (rows, N=207 genes). The columns show MSCs treated with either control vector, EWS-FLI1, wild-type FLI1, or BAF47-FLI1. Expression values were normalized by row. (Right) Example RNA-seq tracks over selected genes.

FIG. 6. Wild-type FLI1 is not able to activate EWS-FLI1 target genes associated with GGAA repeat microsatellites.

(FIG. 6A) Immunoblotting shows lentiviral expression of wild-type FLI1 and EWS-FLI1 in mesenchymal stem cells used in the experiments shown in FIG. 3A-B.

(FIG. 6B) Pie chart shows annotations of MACS-called peaks for FLI1 ChIP-seq in mesenchymal stem cells infected with wild-type FLI1 using Homer.

(FIG. 6C) Wild-type FLI1 can bind and recruit BAF complexes at non-repeat canonical ETS binding sites. Composite plots show ChIP-seq levels for FLI1 (Left) and BAF155 (Right) in control MSCs (black) and MSCs infected with either EWS-FLI1 (purple) or wild-type FLI1 (red). The x-axis represents a 10 kb window centered on EWS-FLI1 non-repeat canonical ETS binding sites in Ewing sarcoma.

(FIG. 6D) Representative example of wild-type FLI1, EWS-FLI1, BAF155 and H3K27ac ChIP-seq at a GGAA repeat enhancer near NKX2-2 in control MSCs and MSCs infected with either EWS-FLI1 or wild-type FLI1 in the experiments shown in FIG. 3A-B. The region of interest is shown in light gray.

(FIG. 6E) Wild-type FLI1 is not able to induce the EWS-FLI1 target gene signature. RTqPCR for selected EWS-FLI1 target genes associated with GGAA repeats after infection of MSCs with either control vector, EWS-FLI1, or wild-type FLI1.

FIG. 7. The fusion protein BAF47-FLI1 is not able to bind and activate EWS-FLI1 target genes associated with GGAA repeat microsatellites.

(FIG. 7A) Immunoblotting shows lentiviral expression of EWS-FLI1 and BAF47-FLI1 fusion protein in mesenchymal stem cells used in the experiments shown in FIG. 3G-H.

(FIG. 7B) Immunofluorescence staining using FLI1 antibodies in infected MSC shows EWSFLI1 and BAF47-FLI1 nuclear localization. Nuclei are counterstained with DAPI.

(FIG. 7C) Pie chart shows annotations of MACS-called peaks for FLI1 ChIP-seq in mesenchymal stem cells infected with BAF47-FLI1 using Homer.

(FIG. 7D) Composite plot shows increased chromatin accessibility detected by ATAC-seq at the top 10,000 BAF47-FLI1-bound sites in mesenchymal stem cells infected with BAF47-FLI1. The x-axis represents a 2 kb window centered on BAF47-FLI1 binding sites.

(FIG. 7E) Composite plot shows FLI1 ChIP-seq signals at GGAA repeat enhancer elements in control MSCs and MSCs expressing EWS-FLI1 or BAF47-FLI1 fusion protein. The x axis represents a 10 kb window centered on EWS-FLI1-bound GGAA repeat enhancer sites. BAF47-FLI1 is not able to stably bind at these sites compared to EWS-FLI1.

(FIG. 7F) Heatmaps of FLI1 and H3K27ac ChIP-seq and ATAC-seq signal densities in MSCs infected with either control vector, EWS-FLI1 or BAF47-FLI1 fusion protein. 10 kb windows in each panel are centered on EWS-FLI1-bound GGAA repeat enhancer sites (N=812).

(FIG. 7G) BAF47-FLI1 fusion protein is not able to induce the EWS-FLI1 target gene signature. RT-qPCR for selected EWS-FLI1 target genes associated with GGAA repeats after infection of MSCs with either control vector, EWS-FLI1, or BAF47-FLI1.

FIG. 8. Fusion to EWSR1 confers FLI1 multimerization and phase transition properties.

(FIG. 8A) Endogenous wild-type EWSR1 strongly interacts with EWS-FLI1 compared to wildtype FLI1. Immunoblots of whole cell extract and anti-V5 immunoprecipitates from 293T cells transfected with either control vector, V5-FLI1, or V5-EWS-FLI1.

(FIG. 8B) Proteomic mass spectrometry of IgG and anti-EWSR1 immunoprecipitations performed in SK-N-MC cells indicates that EWSR1 interacts with FET family members TAF15 and FUS, EWS-FLI1 and BAF complex subunits. Table shows the number of unique peptides in each condition. Members of the PBAF complex are not detectable in these experiments.

(FIG. 8C-E) EWS-FLI1 has a strong ability to precipitate in presence of b-isox compared to wild-type FLI1.

(FIG. 8C) EWS-FLI1 precipitates in presence of 100 uM b-isox in Ewing sarcoma cell lysates.

(FIG. 8D) EWS-FLI1 precipitates upon treatment with b-isox in a dose-dependent manner in Ewing sarcoma cell lysates.

(FIG. 8E) Lentivirally expressed EWS-FLI1 but not wild-type FLI1 precipitates upon treatment with b-isox in U2OS osteosarcoma cell lysates.

(FIG. 8F-G) In vitro sedimentation assays from bacterially expressed and purified EWS-FLI1 or wild-type FLI1.

(FIG. 8F) Quantification of two independent experiments.

(FIG. 8G) Representative examples of in vitro sedimentation assays. The GST tag is cleaved as part of the assay and is used as a soluble internal control.

(FIG. 8H-J) EWSR1 is recruited to EWS-FLI1 bound GGAA repeat enhancers in Ewing Sarcoma. (4H) Example ChIP-seq tracks showing co-occupancy of EWS-FLI1 and HAEWSR1 at GGAA repeat enhancers in A673 cells. Regions of co-occupancy are highlighted in light gray.

(FIG. 8I) Composite plot shows HA-EWSR1 binding at EWS-FLI1 GGAA repeat enhancers in A673 cells. A 10 kb window centered on EWS-FLI1-bound repeat enhancer is shown.

(FIG. 8J) ChIP-qPCR experiments validate HA-EWSR1 binding at EWS-FLI1 GGAA repeat enhancers associated with CCND1, SOX2, NR0B1 and LINC00221, but not a control region near MYT1.

FIG. 9. Loss of EWS-FLI1 results in a decreased occupancy of wild-type EWSR1 at GGAA repeats enhancer elements in Ewing Sarcoma.

(FIG. 9A) Co-immunoprecipitation experiments show interaction of the GFP-EWSR1 fusion protein with endogenous EWSR1 in infected U2OS cells.

(FIG. 9B) EWS-FLI1 precipitates with b-isox in absence of RNA in Ewing sarcoma cell lysates. (Left) Immunoblotting for EWS-FLI1 and EWSR1. In comparison, EWSR1 precipitation decreases in presence of RNase. (Top Right Inset) Representative example of total RNA detection in an agarose gel.

(FIG. 9C) Immunoblots of nuclear extract and anti-EWSR1 immunoprecipitations in either control or RNase A treated SK-N-MC lysates. Interactions between EWSR1, EWS-FLI1 and BAF complexes (BRG1) are maintained.

(FIG. 9D) Immunoblots of whole cell extract and anti-HA immunoprecipitates from 293T cells transfected with either control vector or HA-EWS-FLI1 in absence or presence of RNase A. Interactions between EWS-FLI1, wild-type EWSR1 and BRG1 are maintained.

(FIG. 9E) SDS-PAGE of purified recombinant GST-EWS-FLI1 and GST-FLI1 stained with Coomassie blue. The full-length proteins are indicated by a blue line. Degradation products are visible under the full-length purified proteins.

(FIG. 9F) Confocal imaging of immunofluorescence using anti-V5 antibody shows a dotted pattern for V5-EWS-FLI1 and a more diffuse pattern for V5-FLI1 in MSC cells. Nuclei are counterstained with DAPI.

(FIG. 9G) Immunofluorescence using anti-HA antibody in A673 cells shows nuclear localization of HA-EWSR1. Nuclei are counterstained with DAPI.

(FIG. 9H) Immunoblots of total cell extract and anti-HA immunoprecipitations in A673 cells infected with either control vector or HA-EWSR1. EWSR1 interacts with endogenous EWS-FLI1 and BAF complexes (BRG1).

(FIG. 9I) Heatmaps of HA ChIP-seq signal densities in A673 cells infected with either control vector or HA-EWSR1. 10 kb windows in each panel are centered on EWS-FLI1-bound GGAA repeat enhancer sites (N=812).

(FIG. 9J) Additional Representative example of EWS-FLI1 and HA-EWSR1 co-occupancy at a GGAA repeat enhancer associated with KIT in A673 cells infected with HA-EWSR1 in the experiments shown in FIG. 4H-I. The region of interest is shown in light gray.

(FIG. 9K) ChIP-qPCR experiments show decreased occupancy of HA-EWSR1 at GGAA repeats upon EWS-FLI1 knock-down in A673 cells infected with HA-EWSR1. CCND1, NR0B1, LINC00221 and SOX2 are GGAA repeat microsatellites activated by EWS-FLI1 in Ewing sarcoma and MYT1 is a negative control region.

FIG. 10. Tyrosine residues in the EWS-FLI1 prion-like domain are necessary for DNA binding at GGAA micro-satellites and enhancer induction.

(FIG. 10A) Schematics of EWSR1, EWS-FLI1 and EWS-FLI1 tyrosine mutant variants used in experiments. Tyrosines (Y) mutated into serines (S) are shown as black bars within the EWS N-terminal prion-like domain. Mutants contained either 12 (YS12) or 37 (YS37) Y to S mutations. See also FIG. 11A for a precise annotation of the EWS prion-like domain.

(FIG. 10B) (Left) Immunoblots show nuclear input levels of EWSR1, BAF proteins and the lentiviral expression of EWS-FLI1, EWS(YS12)-FLI1 or EWS(YS37)-FLI1 mutants in U2OS cells. (Right) Co-immunoprecipitation experiments using anti-FLI1 antibodies reveal that the EWS(YS37)-FLI1 mutant exhibits decreased interactions with wild-type EWSR1 and BRG1.

(FIG. 10C) Dose-dependent b-isox precipitation assay after lentiviral expression of either EWSFLI1 or mutants EWS(YS12)-FLI1 or EWS(YS37)-FLI1 in U2OS cells. The strongest decrease is observed for EWS(YS37)-FLI1 mutant.

(FIG. 10D) In vitro sedimentation assay from bacterially expressed and purified EWS(YS37)-FLI1. (Left) Quantification of two independent experiments (Right) Representative examples of in vitro sedimentation assays. The GST tag is cleaved as part of the assay and is used as a soluble internal control.

(FIG. 10E) Heatmaps of FLI1, BAF155 and H3K27ac ChIP-seq signal densities in MSCs treated with either control vector, EWS-FLI1 or EWS(Y37)-FLI1 mutant. 10 kb windows in each panel are centered on EWS-FLI1-bound GGAA repeat enhancer sites (N=812).

(FIG. 10F) ATAC-seq signal intensity indicative of chromatin accessibility at GGAA repeat microsatellites in MSCs infected with either control, EWS-FLI1 or EWS(YS37)-FLI1 mutant. EWS(YS37)-FLI1 mutant does not induce increased accessibility at GGAA repeat enhancers.

(FIG. 10G) Representative example ChIP-seq tracks of FLI1 (EWS-FLI1), H3K27Ac, and ATAC-seq signals over the NKX2-2 locus in MSCs expressing either control, EWS-FLI1, or EWS(YS37)-FLI1 mutant.

(FIG. 10H) Heatmap shows changes in expression detected by RT-qPCR for selected EWSFLI1 target genes associated with GGAA repeats after infection of MSCs with either control vector, EWS-FLI1, EWS(YS12)-FLI1 or EWS(YS37)-FLI1 mutants. EWS(YS37)-FLI1 mutant does not induce the EWS-FLI1 target gene signature.

FIG. 11. Tyrosine residues in the EWS-FLI1 prion-like domain are necessary for DNA binding at GGAA micro-satellites and enhancer induction.

(FIG. 11A) Amino-acid sequence annotation of EWSR1 prion-like domain fused in EWS-FLI1. Legend is found in the figure.

(FIG. 11B) Immunoblotting shows lentiviral expression of EWS-FLI1, EWS(YS12)-FLI1 or EWS(YS37)-FLI1 mutants in mesenchymal stem cells used in the experiments shown in FIG. 10G.

(FIG. 11C) Immunofluorescence staining using FLI1 antibodies in infected MSC shows the nuclear localization of EWS-FLI1, EWS(YS12)-FLI1 or EWS(YS37)-FLI1 mutants. Nuclei are counterstained with DAPI.

(FIG. 11D) EWS(YS37)-FLI1 mutant protein can homodimerize as strongly as its wild-type counterpart. Co-immunoprecipitation experiments using anti-V5 antibodies in transiently transfected HEK-293-T cells co-expressing HA-tagged and V5-tagged wild-type or mutant EWS-FLI1 proteins.

(FIG. 11E) SDS-PAGE of purified recombinant GST-EWS(YS37)-FLI1 stained with Coomassie blue. The full-length proteins are indicated by a blue line. Degradation products are visible under the full-length purified proteins.

(FIG. 11F) Immunoblotting shows lentiviral expression of EWS-FLI1 and EWS(YS37)-FLI1 mutant protein in mesenchymal stem cells used in the experiments shown in FIG. 10D-E-F.

(FIG. 11G) Composite plot shows FLI1 ChIP-seq signals at GGAA repeat enhancer elements in control MSCs and MSCs expressing EWS-FLI1 or EWS(YS37)-FLI1 mutant protein. The x-axis represents a 10 kb window centered on EWS-FLI1-bound GGAA repeat enhancer sites. EWS(YS37)-FLI1 is not able to stably bind at these sites compared to EWS-FLI1.

(FIG. 11H) Heatmap shows ATAC-seq signal densities in MSCs infected with either control vector, EWS-FLI1 or EWS(YS37)-FLI1 mutant protein. 10 kb windows in each panel are centered on EWS-FLI1-bound GGAA repeat enhancer sites (N=812). EWS(YS37)-FLI1 mutant does not induce increased accessibility at GGAA repeat enhancers.

(FIG. 11I) Additional representative examples of ChIP-seq tracks for FLI1 (EWS-FLI1), H3K27Ac, and ATAC-seq signals over GGAA repeats associated genes in MSCs expressing either control, EWS-FLI1, or EWS(YS37)-FLI1 mutant in the experiments shown in FIG. 10D-E-F. The regions of interest are shown in light gray.

FIG. 12. Fusion of fragments of the EWSR1 prion-like domain to the FLI1 Cterminus is sufficient to recapitulate EWS-FLI1 activity.

(FIG. 12A) Schematic representation of EWS-FLI1 prion-like domain mutants used in experiments. SYGQ1 or SYGQ2 fragments are fused to the FLI1 C-terminus.

(FIG. 12B) Dose dependent precipitation assay in presence of b-isox after lentiviral expression of fusion proteins in U2OS cells. SYGQ-FLI1 fusion proteins maintain the ability to precipitate in vitro.

(FIG. 12C) Heatmap shows changes in expression detected by RT-qPCR for selected EWSFLI1 target genes associated with GGAA repeats after infection of MSCs with either control vector, EWS-FLI1, SYGQ1-FLI1 or SYGQ2-FLI1 fusion proteins. Both SYGQ1-FLI1 and SYGQ2-FLI1 mutants induce the EWS-FLI1 target gene signature.

(FIG. 12D) In vitro sedimentation assay from bacterially expressed and purified SYGQ1-FLI1. (Top) Representative examples of in vitro sedimentation assays. The GST tag is cleaved as part of the assay and is used as a soluble internal control. (Bottom) Quantification of two independent experiments.

(FIG. 12E) ATAC-seq signal intensity indicative of chromatin accessibility at GGAA repeat microsatellites in MSCs infected with either control, EWS-FLI1 or the SYGQ2-FLI1 fusion protein. Addition of SYGQ2 to the FLI1 C-terminus (SYGQ2-FLI1) is sufficient to induce increased accessibility at GGAA repeat enhancers.

(FIG. 12F) Heatmaps of FLI1, BAF155 and H3K27ac ChIP-seq signal densities in MSCs treated with either control vector, EWS-FLI1 or the SYGQ2-FLI1 fusion protein. 10 kb windows in each panel are centered on EWS-FLI1-bound GGAA repeat enhancer sites (N=812).

(FIG. 12G) Example ChIP-seq tracks of FLI1 (EWS-FLI1), H3K27Ac, and ATAC-seq signals over the NKX2-2 locus in MSCs expressing either control, EWS-FLI1, or the SYGQ2-FLI1 fusion protein.

(FIG. 12H) Principal component analysis (PCA) plot showing PC1 for the 207 target genes associated with EWS-FLI1 GGAA repeats sites (x-axis) and PC1 for the 158 remaining differentially expressed genes in EWS-FLI1 expressing cells (y-axis). RNA-seq datasets are from MSCs infected with either control vector, EWS-FLI1, EWS(YS37)-FLI1, the SYGQ2-FLI1 fusion protein.

FIG. 13. Internal deletions in the EWS-FLI1 prion-like domain do not abrogate its ability to activate target genes associated with GGAA repeats

(FIG. 13A) Schematic representation of EWS-FLI1 deletion mutants used in the experiments. SYGQ1, SYGQ2 or both were deleted from the EWS-FLI1 prion-like domain.

(FIG. 13B) Immunoblotting shows lentiviral expression of V5-EWS-FLI1 or the V5-tagged EWS-FLI1 mutants in mesenchymal stem cells used in the experiments.

(FIG. 13C) Immunofluorescence staining using anti-V5 antibodies in infected MSC shows the nuclear localization of V5-EWS-FLI1 and the V5-tagged EWS-FLI1 mutants. Nuclei are counterstained with DAPI.

(FIG. 13D) Co-immunoprecipitation experiments using anti-V5 antibodies show that the EWSFLI1 mutants maintain interactions with wild-type EWSR1 and BRG1 in transiently transfected HEK-293-T cells.

(FIG. 13E) EWS-FLI1 prion-like deletion mutants maintain a significant ability to precipitate in vitro. Dose dependent precipitation assay in presence of b-isox in infected U2OS cell lysates. * Indicates endogenous wild-type FLI1 in U2OS, which does not precipitate in these conditions.

(FIG. 13F) Heatmap shows changes in expression detected by RT-qPCR for selected EWSFLI1 target genes associated with GGAA repeats after infection of MSCs with either control vector, EWS-FLI1, or EWS-FLI1 prion-like deletion mutants. All three mutants induce the EWS-FLI1 target gene signature.

(FIG. 13G) (Top) Co-immunoprecipitation experiments using anti-V5 antibodies show that the EWS-FLI1 mutants SYGQ1-FLI1 and SYGQ2-FLI1 maintain interactions with wild-type EWSR1 and BRG1. (Bottom) Immunoblots show nuclear input levels of EWSR1, BRG1 and the lentiviral expression of V5-tagged SYGQ1-FLI1, SYGQ2-FLI1 or EWS-FLI1 in U2OS nuclear extracts.

(FIG. 13H) SDS-PAGE of purified recombinant GST-SYGQ1-FLI1 stained with Coomassie blue. The full-length proteins are indicated by a blue line. Degradation products are visible under the full-length purified proteins.

(FIG. 13I) Rescue experiments with EWS-FLI1 mutant proteins. (Left) Representative cell images and (Right) Cell viability assays (Cell-titer Glo) in SK-N-MC cells treated with either shGFP or shEWS-FLI1 knockdown and induced to express GFP, full size EWSFLI1, EWS(YS37)-FLI1 mutant or the SYGQ2-FLI1 fusion protein. SKNMC cells were infected with inducible pINDUCER lentiviral constructs for GFP, full size EWS-FLI1, EWS(YS37)-FLI1 mutant or the SYGQ2-FLI1 fusion protein and selected with neomycin. This was followed by lentiviral infection with either shGFP or shEWS-FLI1 knockdown and induction of pINDUCER expression with 5 ng/mL doxycycline. *** Indicates p value <0.001.

FIG. 14. Model illustrating the mechanisms that allow EWS-FLI1 binding at GGAA repeat microsatellites followed by enhancer and target gene activation in Ewing sarcoma.

(FIG. 14A) In presence of EWS-FLI1, multimerization is required for stable binding at GGAA repeats and recruitment of RAF complexes.

(FIG. 14B) wild-type FLI1 cannot stably bind at GGAA repeats.

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

Several studies have shown that EWS-FLI1 is necessary for Ewing sarcoma tumorigenicity (Herrero-Martin et al., 2011) and is sufficient for transformation of mesenchymal stem cells (MSCs) (Riggi et al., 2005; Riggi et al., 2008). More recently EWS-FLI1 has been shown to be a major determinant of genome-wide chromatin states in Ewing sarcoma (Riggi et al., 2014; Tomazou et al., 2015). Strikingly, EWS-FLI1 is able to activate a large set of target genes by operating as a pioneer factor at GGAA microsatellite repeats and inducing active enhancers de novo starting from a closed chromatin conformation (Gangwal et al., 2008; Guillon et al., 2009; Patel et al., 2012; Riggi et al., 2014).

The mammalian SWI/SNF (or BAF, for BRG1/BRM-associated factor) complex is an ATP-dependent chromatin remodeler composed of 12-15 subunits that regulates genomic architecture and DNA accessibility (Kadoch and Crabtree, 2015). Recent exome sequencing studies have revealed that the genes encoding BAF complex subunits are recurrently mutated in over 20% of human cancers (Kadoch et al., 2013). Interestingly, specific subunits appear to be mutated in specific cancer subtypes, suggesting their tissue-specific protective functions. For example, SMARCB1 (BAF47/INI1/hSNF5) is mutated in malignant rhabdoid tumor (MRT), atypical teratoid/rhabdoid tumor (AT/RT), and epitheliod sarcomas nearly exclusively; the SS18 gene is translocated to produce the SS18-SSX fusion protein which is the hallmark pathogenic event in synovial sarcoma (Kadoch and Crabtree, 2015; Kadoch et al., 2013; Roberts et al., 2002; Versteege et al., 1998). The high frequency of alterations in BAF complex subunits across a range of tumor types points to their critical role in controlling chromatin architecture and gene expression in cancer (Kadoch and Crabtree, 2015).

It has been determined herein that BAF complexes interact with the wild-type protein EWSR1 in several cell types and with the EWS-FLI1 fusion protein in Ewing sarcoma using an unbiased mass spectrometry approach. The BAF complex is specifically recruited by EWS-FLI1 to tumor-specific GGAA repeat microsatellites and is necessary for the activation of target genes. Remarkably, the ability to recruit BAF complexes and activate enhancers de novo at these repeat sites is a neomorphic property of EWS-FLI1 that depends on tyrosine residues in the EWSR1 prion-like domain which are necessary for its interaction with wild-type EWSR1 and for its phase transition properties in vitro. These observations expand the set of human cancers in which BAF complex mistargeting contributes to oncogenesis beyond settings in which BAF complex genes themselves are mutated, and show that recruitment via a prion-like domain is a powerful means of re-targeting critical chromatin regulatory complexes to tumor-specific loci. Accordingly, the present invention relates, in part, to methods and agents for treating Ewing sarcoma by modulating the interaction between EWS-FLI1 and BAF complexes.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

The term “SWI/SNF complex” refers to SWItch/Sucrose Non-Fermentable, a nucleosome remodeling complex found in both eukaryotes and prokaryotes (Neigeborn Carlson (1984) Genetics 108:845-858; Stern et al. (1984) J. Mol. Biol. 178:853-868). The SWI/SNF complex was first discovered in the yeast, Saccharomyces cerevisiae, named after yeast mating types switching (SWI) and sucrose nonfermenting (SNF) pathways (Workman and Kingston (1998) Annu Rev Biochem. 67:545-579; Sudarsanam and Winston (2000) Trends Genet. 16:345-351). It is a group of proteins comprising, at least, SWI1, SWI2/SNF2, SWI3, SWI5, and SWI6, as well as other polypeptides (Pazin and Kadonaga (1997) Cell 88:737-740). A genetic screening for suppressive mutations of the SWI/SNF phenotypes identified different histones and chromatin components, suggesting that these proteins were possibly involved in histone binding and chromatin organization (Winston and Carlson (1992) Trends Genet. 8:387-391). Biochemical purification of the SWI/SNF2p in S. cerevisiae demonstrated that this protein was part of a complex containing an additional 11 polypeptides, with a combined molecular weight over 1.5 MDa. The SWI/SNF complex contains the ATPase Swi2/Snf2p, two actin-related proteins (Arp7p and Arp9) and other subunits involved in DNA and protein-protein interactions. The purified SWI/SNF complex was able to alter the nucleosome structure in an ATP-dependent manner (Workman and Kingston (1998), supra; Vignali et al. (2000) Mol Cell Biol. 20:1899-1910). The structures of the SWI/SNF and RSC complexes are highly conserved but not identical, reflecting an increasing complexity of chromatin (e.g., an increased genome size, the presence of DNA methylation, and more complex genetic organization) through evolution. For this reason, the SWI/SNF complex in higher eukaryotes maintains core components, but also substitute or add on other components with more specialized or tissue-specific domains. Yeast contains two distinct and similar remodeling complexes, SWI/SNF and RSC (Remodeling the Structure of Chromatin). In Drosophila, the two complexes are called BAP (Brahma Associated Protein) and PBAP (Polybromo-associated BAP) complexes. The human analogs are BAF (Brg1 Associated Factors, or SWI/SNF-A) and PBAF (Polybromo-associated BAF, or SWI/SNF-B). As shown in FIG. 9, the BAF complex comprises, at least, BAF250A (ARID1A), BAF250B (ARID1B), BAF57 (SMARCE1), BAF190/BRM (SMARCA2), BAF47 (SMARCB1), BAF53A (ACTL6A), BRG1/BAF190 (SMARCA4), BAF155 (SMARCC1), and BAF170 (SMARCC2). The PBAF complex comprises, at last, BAF200 (ARID2), BAF180 (PBRM1), BRD7, BAF45A (PHF10), BRG1/BAF190 (SMARCA4), BAF155 (SMARCC1), and BAF170 (SMARCC2). As in Drosophila, human BAF and PBAF share the different core components BAF47, BAF57, BAF60, BAF155, BAF170, BAF45 and the two actins b-Actin and BAF53 (Mohrmann and Verrijzer (2005) Biochim Biophys Acta. 1681:59-73). The central core of the BAF and PBAF is the ATPase catalytic subunit BRG1/hBRM, which contains multiple domains to bind to other protein subunits and acetylated histones. For a summary of different complex subunits and their domain structure, see Tang et al. (2010) Prog Biophys Mol Biol. 102:122-128 (e.g., FIG. 3), Hohmann and Vakoc (2014) Trends Genet. 30:356-363 (e.g., FIG. 1), and Kadoch and Crabtree (2015) Sci. Adv. 1:e1500447. For chromatin remodeling, the SWI/SNF complex use the energy of ATP hydrolysis to slide the DNA around the nucleosome. The first step consists in the binding between the remodeler and the nucleosome. This binding occurs with nanomolar affinity and reduces the digestion of nucleosomal DNA by nucleases. The 3-D structure of the yeast RSC complex was first solved and imaged using negative stain electron microscopy (Asturias et al. (2002) Proc Nall Acad Sci USA 99:13477-13480). The first Cryo-EM structure of the yeast SWI/SNF complex was published in 2008 (Dechassa et al. 2008). DNA footprinting data showed that the SWI/SNF complex makes close contacts with only one gyre of nucleosomal DNA. Protein crosslinking showed that the ATPase SWI2/SNF2p and Swi5p (the homologue of Ini1p in human), Snf6, Swi29, Snf11 and Sw82p (not conserved in human) make close contact with the histones. Several individual SWI/SNF subunits are encoded by gene families, whose protein products are mutually exclusive in the complex (Wu et al. (2009) Cell 136:200-206). Thus, only one paralog is incorporated in a given SWI/SNF assembly. The only exceptions are BAF155 and BAF170, which are always present in the complex as homo- or hetero-dimers.

Combinatorial association of SWI/SNF subunits could in principle give rise to hundreds of distinct complexes, although the exact number has yet to be determined (Wu et al. (2009), supra). Genetic evidence suggests that distinct subunit configurations of SWI/SNF are equipped to perform specialized functions. As an example, SWI/SNF contains one of two ATPase subunits, BRG1 or BRM/SMARCA2, which share 75% amino acid sequence identity (Khavari et al. (1993) Nature 366:170-174). While in certain cell types BRG1 and BRM can compensate for loss of the other subunit, in other contexts these two ATPases perform divergent functions (Strobeck et al. (2002) J Biol Chem. 277:4782-4789; Hoffman et al. (2014) Proc Natl Acad Sci USA. 111:3128-3133). In some cell types, BRG1 and BRM can even functionally oppose one another to regulate differentiation (Flowers et al. (2009) J Biol Chem. 284:10067-10075). The functional specificity of BRG1 and BRM has been linked to sequence variations near their N-terminus, which have different interaction specificities for transcription factors (Kadam and Emerson (2003) Mol Cell. 11:377-389). Another example of paralogous subunits that form mutually exclusive SWI/SNF complexes are ARID1A/BAF250A, ARID1B/BAF250B, and ARID2/BAF200. ARID1A and ARID1B share 60% sequence identity, but yet can perform opposing functions in regulating the cell cycle, with MYC being an important downstream target of each paralog (Nagl et al. (2007) EMBO J. 26:752-763). ARID2 has diverged considerably from ARID1A/ARID1B and exists in a unique SWI/SNF assembly known as PBAF (or SWI/SNF-B), which contains several unique subunits not found in ARID1A/B-containing complexes. The composition of SWI/SNF can also be dynamically reconfigured during cell fate transitions through cell type-specific expression patterns of certain subunits. For example, BAF53A/ACTL6A is repressed and replaced by BAF53B/ACTL6B during neuronal differentiation, a switch that is essential for proper neuronal functions in vivo (Lessard et al. (2007) Neuron 55:201-215). These studies stress that SWI/SNF in fact represents a collection of multi-subunit complexes whose integrated functions control diverse cellular processes, which is also incorporated in the scope of definitions of the instant disclosure. Two recently published meta-analyses of cancer genome sequencing data estimate that nearly 20% of human cancers harbor mutations in one (or more) of the genes encoding SWI/SNF (Kadoch et al. (2013) Nat Genet. 45:592-601; Shain and Pollack (2013) PLoS One. 8:e55119). Such mutations are generally loss-of-function, implicating SWI/SNF as a major tumor suppressor in diverse cancers. Specific SWI/SNF gene mutations are generally linked to a specific subset of cancer lineages: SNF5 is mutated in malignant rhabdoid tumors (MRT), PBRM1/BAF180 is frequently inactivated in renal carcinoma, and BRG1 is mutated in non-small cell lung cancer (NSCLC) and several other cancers. In the instant disclosure, the scope of “SWI/SNF complex” may cover at least one fraction or the whole complex (e.g., some or all subunit proteins/other components), either in the human BAF/PBAF forms or their homologs/orthologs in other species (e.g., the yeast and drosophila forms described herein). Preferably, a “SWI/SNF complex” described herein contains at least part of the full complex bio-functionality, such as binding to other subunits/components, binding to DNA/histone, catalyzing ATP, promoting chromatin remodeling, etc.

The term “BAF complex” refers to at least one type of mammalian SWI/SNF complexes. Its nucleosome remodeling activity can be reconstituted with a set of four core subunits (BRG1/SMARCA4, SNF5/SMARCB1, BAF155/SMARCC1, and BAF170/SMARCC2), which have orthologs in the yeast complex (Phelan et al. (1999) Mol Cell. 3:247-253). However, mammalian SWI/SNF contains several subunits not found in the yeast counterpart, which can provide interaction surfaces for chromatin (e.g. acetyl-lysine recognition by bromodomains) or transcription factors and thus contribute to the genomic targeting of the complex (Wang et al. (1996) EMBO J. 15:5370-5382; Wang et al. (1996) Genes Dev. 10:2117-2130; Nie et al. (2000)). A key attribute of mammalian SWI/SNF is the heterogeneity of subunit configurations that can exist in different tissues and even in a single cell type (e.g., as BAF, PBAF, neural progenitor BAF (npBAF), neuron BAF (nBAF), embryonic stem cell BAF (esBAF), etc.). In some embodiments, the BAF complex described herein refers to one type of mammalian SWI/SNF complexes, which is different from PBAF complexes.

The term “PBAF complex” refers to one type of mammalian SWI/SNF complexes originally known as SWI/SNF-B. It is highly related to the BAF complex and can be separated with conventional chromatographic approaches. For example, human BAF and PBAF complexes share multiple identical subunits (such as BRG, BAF170, BAF155, BAF60, BAF57, BAF53, BAF45, actin, SS18, and hSNF5/INI1). However, while BAF contains BAF250 subunit, PBAF contains BAF180 and BAF200, instead (Lemon et al. (2001) Nature 414:924-998; Yan et al. (2005) Genes Dev. 19:1662-1667). Moreover, they do have selectivity in regulating interferon-responsive genes (Yan et al. (2005), supra, showing that BAF200, but not BAF180, is required for PBAF to mediate expression of IFITM1 gene induced by IFN-α, while the IFITM3 gene expression is dependent on BAF but not PBAF). Due to these differences, PBAF, but not BAF, was able to activate vitamin D receptor-dependent transcription on a chromatinzed template in vitro (Lemon et al. (2001), supra). The 3-D structure of human PBAF complex preserved in negative stain was found to be similar to yeast RSC but dramatically different from yeast SWI/SNF (Leschziner et al. (2005) Structure 13:267-275).

The term “BRG” or “BRG1/BAF190 (SMARCA4)” refers to a subunit of the SWI/SNF complex, which can be find in either BAF or PBAF complex. It is an ATP-depedendent helicase and a transcription activator, encoded by the SMARCA4 gene. BRG1 can also bind BRCA1, as well as regulate the expression of the tumorigenic protein CD44. BRG1 is important for development past the pre-implantation stage. Without having a functional BRG1, exhibited with knockout research, the embryo will not hatch out of the zona pellucida, which will inhibit implantation from occurring on the endometrium (uterine wall). BRG1 is also crucial to the development of sperm. During the first stages of meiosis in spermatogenesis there are high levels of BRG1. When BRG1 is genetically damaged, meiosis is stopped in prophase 1, hindering the development of sperm and would result in infertility. More knockout research has concluded BRG1's aid in the development of smooth muscle. In a BRG1 knockout, smooth muscle in the gastrointestinal tract lacks contractility, and intestines are incomplete in some cases. Another defect occurring in knocking out BRG1 in smooth muscle development is heart complications such as an open ductus arteriosus after birth (Kim et al. (2012) Development 139:1133-1140; Zhang et al. (2011) Mol. Cell. Biol. 31:2618-2631). Mutations in SMARCA4 were first recognized in human lung cancer cell lines (Medina et al. (2008) Hum. Mut. 29:617-622). Later it was recognized that mutations exist in a significant frequency of medulloblastoma and pancreatic cancers among other tumor subtypes (Jones et al. (2012) Nature 488:100-105; Shain et al. (2012) Proc Natl Acad Sci USA 109:E252-E259; Shain and Pollack (2013), supra). Mutations in BRG1 (or SMARCA4) appear to be mutually exclusive with the presence of activation at any of the MYC-genes, which indicates that the BRG1 and MYC proteins are functionally related. Another recent study demonstrated a causal role of BRG1 in the control of retinoic acid and glucocorticoid-induced cell differentiation in lung cancer and in other tumor types. This enables the cancer cell to sustain undifferentiated gene expression programs that affect the control of key cellular processes. Furthermore, it explains why lung cancer and other solid tumors are completely refractory to treatments based on these compounds that are effective therapies for some types of leukemia (Romero et al. (2012) EMBO Mol. Med. 4:603-616). The role of BRG1 in sensitivity or resistance to anti-cancer drugs had been recently highlighted by the elucidation of the mechanisms of action of darinaparsin, an arsenic-based anti-cancer drugs. Darinaparsin has been shown to induce phosphorylation of BRG1, which leads to its exclusion from the chromatin. When excluded from the chromatin, BRG1 can no longer act as a transcriptional co-regulator. This leads to the inability of cells to express HO-1, a cytoprotective enzyme. BRG1 has been shown to interact with proteins such as ACTL6A, ARID1A, ARID1B, BRCA1, CTNNB1, CBX5, CREBBP, CCNE1, ESR1, FANCA, HSP90B1, ING1, Myc, NR3C1, P53, POLR2A, PHB, SIN3A, SMARCB1, SMARCC1, SMARCC2, SMARCE1, STAT2, STK11, etc.

The term “BRG” or “BRG1/BAF190 (SMARCA4)” is intended to include fragments, variants (e.g., allelic variants), and derivatives thereof. Representative human BRG1(SMARCA4) cDNA and human BRG1 protein sequences are well-known in the art and are publicly available from the National Center for Biotechnology Information (NCBI). For example, seven different human BRG1 isoforms are known. Human BRG1 isoform A (NP_001122321.1) is encodable by the transcript variant 1 (NM_001128849.1), which is the longest transcript. Human BRG1 isoform B (NP_001122316.1 or NP_003063.2) is encodable by the transcript variant 2 (NM_001128844.1), which differs in the 5′ UTR and lacks an alternate exon in the 3′ coding region, compared to the variant 1, and also by the transcript variant 3 (NM_003072.3), which lacks an alternate exon in the 3′ coding region compared to variant 1. Human BRG1 isoform C (NP_001122317.1) is encodable by the transcript variant 4 (NM 001128845.1), which lacks two alternate in-frame exons and uses an alternate splice site in the 3′ coding region, compared to variant 1. Human BRG1 isoform D (NP_001122318.1) is encodable by the transcript variant 5 (NM_001128846.1), which lacks two alternate in-frame exons and uses two alternate splice sites in the 3′ coding region, compared to variant 1. Human BRG1 isoform E (NP_001122319.1) is encodable by the transcript variant 6 (NM_001128847.1), which lacks two alternate in-frame exons in the 3′ coding region, compared to variant 1. Human BRG1 isoform F (NP_001122320.1) is encodable by the transcript variant 7 (NM_001128848.1), which lacks two alternate in-frame exons and uses an alternate splice site in the 3′ coding region, compared to variant 1. Nucleic acid and polypeptide sequences of BRG1 orthologs in organisms other than humans are well known and include, for example, chimpanzee BRG1 (XM_016935029.1 and XP_016790518.1, XM_016935038.1 and XP_016790527.1, XM_016935039.1 and XP_016790528.1, XM_016935036.1 and XP_016790525.1, XM_016935037.1 and XP_016790526.1, XM_016935041.1 and XP_016790530.1, XM_016935040.1 and XP_016790529.1, XM_016935042.1 and XP_016790531.1, XM_016935043.1 and XP_016790532.1, XM_016935035.1 and XP_016790524.1, XM_016935032.1 and XP_016790521.1, XM_016935033.1 and XP_016790522.1, XM_016935030.1 and XP_016790519.1, XM_016935031.1 and XP_016790520.1, and XM_016935034.1 and XP_016790523.1), Rhesus monkey BRG1 (XM_015122901.1 and XP_014978387.1, XM_015122902.1 and XP_014978388.1, XM_015122903.1 and XP_014978389.1, XM_015122906.1 and XP_014978392.1, XM_015122905.1 and XP_014978391.1, XM_015122904.1 and XP_014978390.1, XM_015122907.1 and XP_014978393.1, XM_015122909.1 and XP_014978395.1, and XM_015122910.1 and XP_014978396.1), dog BRG1 (XM_014122046.1 and XP_013977521.1, XM_014122043.1 and XP_013977518.1, XM_014122042.1 and XP_013977517.1, XM_014122041.1 and XP_013977516.1, XM_014122045.1 and XP_013977520.1, and XM_014122044.1 and XP_013977519.1), cattle BRG1 (NM_001105614.1 and NP_001099084.1), rat BRG1 (NM_134368.1 and NP_599195.1).

Anti-BRG1 antibodies suitable for detecting BRG1 protein are well-known in the art and include, for example, MABE1118, MABE121, MABE60, and 07-478 (poly- and mono-clonal antibodies from EMD Millipore, Billerica, Mass.), AM26021PU-N, AP23972PU-N, TA322909; TA322910, TA327280, TA347049, TA347050, TA347851, and TA349038 (antibodies from OriGene Technologies, Rockville, Md.), NB100-2594, AF5738, NBP2-22234, NBP2-41270, NBP1-51230, and NBP1-40379 (antibodes from Novus Biologicals, Littleton, Colo.), ab110641, ab4081, ab215998, ab108318, ab70558, ab118558, ab133257, ab92496, ab196535, and ab196315 (antibodies from AbCam, Cambridge, Mass.), Cat #: 720129, 730011, 730051, MA1-10062, PA5-17003, and PA5-17008 (antibodies from ThermoFisher Scientific, Waltham, Mass.), GTX633391, GTX32478, GTX31917, GTX16472, and GTX50842 (antibodies from GeneTex, Irvine, Calif.), antibody 7749 (ProSci, Poway, Calif.), Brg-1 (N-15), Brg-1 (N-15) X, Brg-1 (H-88), Brg-1 (H-88) X, Brg-1 (P-18), Brg-1 (P-18) X, Brg-1 (G-7), Brg-1 (G-7) X, Brg-1 (H-10), and Brg-1 (H-10) X (antibodies from Santa Cruz Biotechnology, Dallas, Tex.), antibody of Cat. AF5738 (R&D Systmes, Minneapolis, Minn.), etc. In addition, reagents are well-known for detecting BRG1 expression. Moreover, multiple siRNA, shRNA, CRISPR constructs for reducing BRG1 Expression can be found in the commercial product lists of the above-referenced companies. PFI 3 is a known small molecule inhibitor of polybromo 1 and BRG1 (e.g., Cat. B7744 from APE×BIO, Houston, Tex.). It is to be noted that the term can further be used to refer to any combination of features described herein regarding BRG1 molecules. For example, any combination of sequence composition, percentage identify, sequence length, domain structure, functional activity, etc. can be used to describe an BRG1 molecule of the present invention.

The term “BRM” or “BRM/BAF190 (SMARCA2)” refers to a subunit of the SWI/SNF complex, which can be found in either BAF or PBAF complexes. It is an ATP-depedendent helicase and a transcription activator, encoded by the SMARCA2 gene. The catalytic core of the SWI/SNF complex can be either of two closely related ATPases, BRM or BRG1, with the potential that the choice of alternative subunits is a key determinant of specificity. Instead of impeding differentiation as was seen with BRG1 depletion, depletion of BRM caused accelerated progression to the differentiation phenotype. BRM was found to regulate genes different from those as BRG1 targets and be capable of overriding BRG1-dependent activation of the osteocalcin promoter, due to its interaction with different ARID family members (Flowers et al. (2009), supra). The known binding partners for BRM include, for example, ACTL6A, ARID1B, CEBPB, POLR2A, Prohibitin, SIN3A, SMARCB1, and SMARCC1.

The term “BRM” or “BRM/BAF190 (SMARCA2)” is intended to include fragments, variants (e.g., allelic variants), and derivatives thereof. Representative human BRM (SMARCA2) cDNA and human BRM protein sequences are well-known in the art and are publicly available from the National Center for Biotechnology Information (NCBI). For example, seven different human BRM isoforms are known. Human BRM isoform A (NP_003061.3 or NP_001276325.1) is encodable by the transcript variant 1 (NM_003070.4), which is the longest transcript, or the transcript variant 3 (NM_001289396.1), which differs in the 5′ UTR, compared to variant 1. Human BRM isoform B (NP_620614.2) is encodable by the transcript variant 2 (NM_139045.3), which lacks an alternate in-frame exon in the coding region, compared to variant 1. Human BRM isoform C (NP_001276326.1) is encodable by the transcript variant 4 (NM_001289397.1), which uses an alternate in-frame splice site and lacks an alternate in-frame exon in the 3′ coding region, compared to variant 1. Human BRM isoform D (NP_001276327.1) is encodable by the transcript variant 5 (NM_001289398.1), which differs in the 5′ UTR, lacks a portion of the 5′ coding region, and initiates translation at an alternate downstream start codon, compared to variant 1. Human BRM isoform E (NP_001276328.1) is encodable by the transcript variant 6 (NM_001289399.1), which differs in the 5′ UTR, lacks a portion of the 5′ coding region, and initiates translation at an alternate downstream start codon, compared to variant 1. Human BRM isoform F (NP_001276329.1) is encodable by the transcript variant 7 (NM_001289400.1), which differs in the 5′ UTR, lacks a portion of the 5′ coding region, and initiates translation at an alternate downstream start codon, compared to variant 1. Nucleic acid and polypeptide sequences of BRM orthologs in organisms other than humans are well known and include, for example, chimpanzee BRM (XM_016960529.1 and XP_016816018.1), dog BRG1 (XM_005615906.2 and XP 005615963.1, XM_845066.4 and XP_850159.1, XM_005615905.2 and XP_005615962.1, XM_005615904.2 and XP_005615961.1, XM_005615903.2 and XP_005615960.1, and XM_005615902.2 and XP_005615959.1), cattle BRM (NM_001099115.2 and NP_001092585.1), rat BRM (NM_001004446.1 and NP_001004446.1).

Anti-BRM antibodies suitable for detecting BRM protein are well-known in the art and include, for example, antibody MABE89 (EMD Millipore, Billerica, Mass.), antibody TA351725 (OriGene Technologies, Rockville, Md.), NBP1-90015, NBP1-80042, NB100-55308, NB100-55309, NB100-55307, and H00006595-M06 (antibodes from Novus Biologicals, Littleton, Colo.), ab15597, ab12165, ab58188, and ab200480 (antibodies from AbCam, Cambridge, Mass.), Cat #: 11966 and 6889 (antibodies from Cell Signaling, Danvers, Mass.), etc. In addition, reagents are well-known for detecting BRM expression. Moreover, multiple siRNA, shRNA, CRISPR constructs for reducing BRM Expression can be found in the commercial product lists of the above-referenced companies. For example, BRM RNAi product H00006595-R02 (Novus Biologicals), CRISPER gRNA products from GenScript, Piscataway, N.J., and other inhibitory RNA products from Origene, ViGene Biosciences (Rockville, Md.), and Santa Cruz. It is to be noted that the term can further be used to refer to any combination of features described herein regarding BRM molecules. For example, any combination of sequence composition, percentage identify, sequence length, domain structure, functional activity, etc. can be used to describe an BRM molecule of the present invention.

The term “BAF250A” or “ARID1A” refers to AT-rich interactive domain-containing protein 1A, a subunit of the SWI/SNF complex, which can be find in BAF but not PBAF complex. In humans there are two BAF250 isoforms, BAF250A/ARID1A and BAF250B/ARID1B. They are thought to be E3 ubiquitin ligases that target histone H2B (Li et al. (2010) Mol. Cell. Biol. 30:1673-1688). ARID1A is highly expressed in the spleen, thymus, prostate, testes, ovaries, small intestine, colon and peripheral leukocytes. ARID1A is involved in transcriptional activation and repression of select genes by chromatin remodeling. It is also involved in vitamin D-coupled transcription regulation by associating with the WINAC complex, a chromatin-remodeling complex recruited by vitamin D receptor. ARID1A belongs to the neural progenitors-specific chromatin remodeling (npBAF) and the neuron-specific chromatin remodeling (nBAF) complexes, which are involved in switching developing neurons from stem/progenitors to post-mitotic chromatin remodeling as they exit the cell cycle and become committed to their adult state. ARID1A also plays key roles in maintaining embryonic stem cell pluripotency and in cardiac development and function (Lei et al. (2012) J. Biol. Chem. 287:24255-24262; Gao et al. (2008) Proc. Natl. Acad. Sci. U.S.A. 105:6656-6661). Loss of BAF250a expression was seen in 42% of the ovarian clear cell carcinoma samples and 21% of the endometrioid carcinoma samples, compared with just 1% of the high-grade serous carcinoma samples. ARID1A deficiency also impairs the DNA damage checkpoint and sensitizes cells to PARP inhibitors (Shen et al. (2015) Cancer Discov. 5:752-767). Human ARID1A protein has 2285 amino acids and a molecular mass of 242045 Da, with at least a DNA-binding domain that can specifically bind an AT-rich DNA sequence, recognized by a SWI/SNF complex at the beta-globin locus, and a C-terminus domain for glucocorticoid receptor-dependent transcriptional activation. ARID1A has been shown to interact with proteins such as SMARCB1/BAF47 (Kato et al. (2002) J. Biol. Chem. 277:5498-505; Wang et al. (1996) EMBO J. 15:5370-5382) and SMARCA4/BRG1 (Wang et al. (1996), supra; Zhao et al. (1998) Cell 95:625-636), etc.

The term “BAF250A” or “ARID1A” is intended to include fragments, variants (e.g., allelic variants), and derivatives thereof. Representative human BAF250A (ARID1A) cDNA and human BAF250A (ARID1A) protein sequences are well-known in the art and are publicly available from the National Center for Biotechnology Information (NCBI). For example, two different human ARID1A isoforms are known. Human ARID1A isoform A (NP_006006.3) is encodable by the transcript variant 1 (NM_006015.4), which is the longer transcript. Human ARID1A isoform B (NP_624361.1) is encodable by the transcript variant 2 (NM_139135.2), which lacks a segment in the coding region compared to variant 1. Isoform B thus lacks an internal segment, compared to isoform A. Nucleic acid and polypeptide sequences of ARID1A orthologs in organisms other than humans are well known and include, for example, chimpanzee ARID1A (XM_016956953.1 and XP_016812442.1, XM_016956958.1 and XP_016812447.1, and XM_009451423.2 and XP_009449698.2), Rhesus monkey ARID1A (XM_015132119.1 and XP_014987605.1, and XM_015132127.1 and XP_014987613.1), dog ARID1A (XM_847453.5 and XP_852546.3, XM_005617743.2 and XP_005617800.1, XM_005617742.2 and XP_005617799.1, XM_005617744.2 and XP_005617801.1, XM_005617746.2 and XP_005617803.1, and XM_005617745.2 and XP_005617802.1), cattle ARID1A (NM_001205785.1 and NP_001192714.1), rat ARID1A (NM_001106635.1 and NP_001100105.1).

Anti-ARID1A antibodies suitable for detecting ARID1A protein are well-known in the art and include, for example, antibody Cat #04-080 (EMD Millipore, Billerica, Mass.), antibodies TA349170, TA350870, and TA350871 (OriGene Technologies, Rockville, Md.), antibodies NBP1-88932, NB 100-55334, NBP2-43566, NB 100-55333, and 1100008289-Q01 (Novus Biologicals, Littleton, Colo.), antibodies ab182560, ab182561, ab176395, and ab97995 (AbCam, Cambridge, Mass.), antibodies Cat #: 12354 and 12854 (Cell Signaling Technology, Danvers, Mass.), antibodies GTX129433, GTX129432, GTX632013, GTX12388, and GTX31619 (GeneTex, Irvine, Calif.), etc. In addition, reagents are well-known for detecting ARID1A expression. For example, multiple clinical tests for ARID1A are available at NIH Genetic Testing Registry (GTR®) (e.g., GTR Test ID: GTR000520952.1 for mental retardation, offered by Centogene AG, Germany). Moreover, multiple siRNA, shRNA, CRISPR constructs for reducing ARID1A Expression can be found in the commercial product lists of the above-referenced companies, such as RNAi products H00008289-R01, H00008289-R02, and H00008289-R03 (Novus Biologicals) and CRISPR products KN301547G1 and KN301547G2 (Origene). Other CRISPR products include sc-400469 (Santa Cruz Biotechnology) and those from GenScript (Piscataway, N.J.). It is to be noted that the term can further be used to refer to any combination of features described herein regarding ARID1A molecules. For example, any combination of sequence composition, percentage identify, sequence length, domain structure, functional activity, etc. can be used to describe an ARID1A molecule of the present invention.

The term “loss-of-function mutation” for BAF250A/ARID1A refers to any mutation in an ARID1A-related nucleic acid or protein that results in reduced or eliminated ARID1A protein amounts and/or function. For example, nucleic acid mutations include single-base substitutions, multi-base substitutions, insertion mutations, deletion mutations, frameshift mutations, missesnse mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of ARID1A. Such mutations reduce or eliminate ARID1A protein amounts and/or function by eliminating proper coding sequences required for proper ARID1A protein translation and/or coding for ARID1A proteins that are non-functional or have reduced function (e.g., deletion of enzymatic and/or structural domains, reduction in protein stability, alteration of sub-cellular localization, and the like). Such mutations are well-known in the art. In addition, a representative list describing a wide variety of structural mutations correlated with the functional result of reduced or eliminated ARID1A protein amounts and/or function is described in the Tables and the Examples.

The term “BAF250B” or “ARID 1B” refers to AT-rich interactive domain-containing protein 1B, a subunit of the SWI/SNF complex, which can be find in BAF but not PBAF complex. ARID1B and ARID1A are alternative and mutually exclusive ARID-subunits of the SWI/SNF complex. Germline mutations in ARID1B are associated with Coffin-Siris syndrome (Tsurusaki et al. (2012) Nat. Genet. 44:376-378; Santen et al. (2012) Nat. Genet. 44:379-380). Somatic mutations in ARID1B are associated with several cancer subtypes, suggesting that it is a tumor suppressor gene (Shai and Pollack (2013) PLoS ONE 8:e55119; Sausen et al. (2013) Nat. Genet. 45:12-17; Shain et al. (2012) Proc. Natl. Acad. Sci. U.S.A. 109:E252-E259; Fujimoto et al. (2012) Nat. Genet. 44:760-764). Human ARID1A protein has 2236 amino acids and a molecular mass of 236123 Da, with at least a DNA-binding domain that can specifically bind an AT-rich DNA sequence, recognized by a SWI/SNF complex at the beta-globin locus, and a C-terminus domain for glucocorticoid receptor-dependent transcriptional activation. ARID1B has been shown to interact with SMARCA4/BRG1 (Hurlstone et al. (2002) Biochem. J. 364:255-264; Inoue et al. (2002) J. Biol. Chem. 277:41674-41685 and SMARCA2/BRM (Inoue et al. (2002), supra).

The term “BAF250B” or “ARID1B” is intended to include fragments, variants (e.g., allelic variants), and derivatives thereof. Representative human BAF250B (ARID1B) cDNA and human BAF250B (ARID B) protein sequences are well-known in the art and are publicly available from the National Center for Biotechnology Information (NCBI). For example, three different human ARID1B isoforms are known. Human ARID1B isoform A (NP_059989.2) is encodable by the transcript variant 1 (NM_017519.2). Human ARID1B isoform B (NP_065783.3) is encodable by the transcript variant 2 (NM_020732.3). Human ARID1B isoform C (NP_001333742.1) is encodable by the transcript variant 3 (NM_001346813.1). Nucleic acid and polypeptide sequences of ARID1B orthologs in organisms other than humans are well known and include, for example, Rhesus monkey ARID1B (XM_015137088.1 and XP_014992574.1), dog ARID1B (XM_014112912.1 and XP_013968387.1), cattle ARID1B (XM_010808714.2 and XP_010807016.1, and XM_015464874.1 and XP_015320360.1), rat ARID1B (XM_017604567.1 and XP_017460056.1).

Anti-ARID1B antibodies suitable for detecting ARID1B protein are well-known in the art and include, for example, antibody Cat # ABE316 (EMD Millipore, Billerica, Mass.), antibody TA315663 (OriGene Technologies, Rockville, Md.), antibodies H00057492-M02, H00057492-M01, NB 100-57485, NBP1-89358, and NB100-57484 (Novus Biologicals, Littleton, CU), antibodies ab57461, ab69571, ab84461, and ab163568 (AbCam, Cambridge, Mass.), antibodies Cat #: PA5-38739, PA5-49852, and PA5-50918 (ThermoFisher Scientific, Danvers, Mass.), antibodies GTX130708, GTX60275, and GTX56037 (GeneTex, Irvine, Calif.), ARID1B (KMN1) Antibody and other antibodies (Santa Cruz Biotechnology), etc. In addition, reagents are well-known for detecting ARID1B expression. For example, multiple clinical tests for ARID1B are available at NIH Genetic Testing Registry (GTR®) (e.g., GTR Test ID: GTR000520953.1 for mental retardation, offered by Centogene AG, Germany). Moreover, multiple siRNA, shRNA, CRISPR constructs for reducing ARID1B Expression can be found in the commercial product lists of the above-referenced companies, such as RNAi products H00057492-R03, H00057492-R01, and H00057492-R02 (Novus Biologicals) and CRISPR products KN301548 and KN214830 (Origene). Other CRISPR products include sc-402365 (Santa Cruz Biotechnology) and those from GenScript (Piscataway, N.J.). It is to be noted that the term can further be used to refer to any combination of features described herein regarding ARID1B molecules. For example, any combination of sequence composition, percentage identify, sequence length, domain structure, functional activity, etc. can be used to describe an ARID1B molecule of the present invention.

The term “loss-of-function mutation” for BAF250B/ARID1B refers to any mutation in an ARID1B-related nucleic acid or protein that results in reduced or eliminated ARID1B protein amounts and/or function. For example, nucleic acid mutations include single-base substitutions, multi-base substitutions, insertion mutations, deletion mutations, frameshift mutations, missesnse mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of ARID1B. Such mutations reduce or eliminate ARID1B protein amounts and/or function by eliminating proper coding sequences required for proper ARID1B protein translation and/or coding for ARID B proteins that are non-functional or have reduced function (e.g., deletion of enzymatic and/or structural domains, reduction in protein stability, alteration of sub-cellular localization, and the like). Such mutations are well-known in the art. In addition, a representative list describing a wide variety of structural mutations correlated with the functional result of reduced or eliminated ARID1B protein amounts and/or function is described in the Tables and the Examples.

The term “EWSR1” refers to EWS RNA binding protein 1, a multifunctional protein that is involved in various cellular processes, including gene expression, cell signaling, and RNA processing and transport. The protein includes an N-terminal transcriptional activation domain and a C-terminal RNA-binding domain. Chromosomal translocations between this gene and various genes encoding transcription factors result in the production of chimeric proteins that are involved in tumorigenesis. These chimeric proteins usually consist of the N-terminal transcriptional activation domain of this protein fused to the C-terminal DNA-binding domain of the transcription factor protein. Mutations in this gene, specifically a t(11;22)(q24;q12) translocation, are known to cause Ewing sarcoma as well as neuroectodermal and various other tumors. Human EWSR1 protein has 656 amino acids and a molecular mass of 68478 Da.

The term “EWSR1” is intended to include fragments, variants (e.g., allelic variants), and derivatives thereof. Representative human EWSR1 cDNA and human EWSR1 protein sequences are well-known in the art and are publicly available from the National Center for Biotechnology Information (NCBI). Human EWSR1 isoforms include the longest isoform 1 (NM_013986.3 and NP_053733.2), and the shorter isoforms 2 (NM_005243.3 and NP_005234.1; which lacks an alternate in-frame exon and uses an alternate in-frame splice site in the coding region, resulting in a shorter isoform compared to isoform 1), 3 (NM_001163285.1 and NP_001156757.1; which lacks an alternate in-frame exon in the coding region, resulting a shorter isoform compared to isoform 1), 4 (NM_001163286.1 and NP_001156758.1; which lacks two alternate in-frame exons and uses an alternate in-frame splice site in the coding region, resulting in a shorter isoform compared to isoform 1), and 5 (NM_001163287.1 and NP_001156759.1; which lacks an alternate in-frame exon in the 5′ coding region, and differs in the 3′ UTR and in the presence and absence of exons in the 3′ coding region, resulting in a shorter isoform with distinct C-terminus compared to isoform 1). Nucleic acid and polypeptide sequences of EWSR1 orthologs in organisms other than humans are well-known and include, for example, chimpanzee EWSR1 (NM_001280490.1 and NP_001267419.1), monkey EWSR1 (NM_001266241.2 and NP_001253170.1), dog EWSR1 (NM_001290126.1 and NP_001277055.1), cattle EWSR1 (1.NM_001109800.2 and NP_001103270.1), and rat EWSR1 (NM_001025632.2 and NP_001020803.2).

Anti-EWSR1 antibodies suitable for detecting EWSR1 protein are well-known in the art and include, for example, antibodies TA305656 and AM31782PU-N(OriGene), antibody Cat #: 11910 (Cell Signaling), antibodies NB200-182 and NBP1-28798, (Novus Biologicals, Littleton, Colo.), etc. In addition, reagents are well-known for detecting EWSR1 expression. Moreover, multiple siRNA, shRNA, CRISPR constructs for reducing EWSR1 expression can be found in the commercial product lists of the above-referenced companies, such as shRNA product # TG313142, and CRISPR products # KN203709 from Origene Technologies (Rockville, Md.), and RNAi products Cat #: 12175 and Cat #: 12216 from Cell Signaling. It is to be noted that the term can further be used to refer to any combination of features described herein regarding EWSR1 molecules. For example, any combination of sequence composition, percentage identify, sequence length, domain structure, functional activity, etc. can be used to describe an EWSR1 molecule of the present invention.

The term “FLI1” refers to Fli-1 proto-oncogene, ETS transcription factor, a transcription factor containing an ETS DNA-binding domain. The gene can undergo a t(11;22)(q24;q 12) translocation with the Ewing sarcoma gene on chromosome 22, which results in a fusion gene that is present in the majority of Ewing sarcoma cases. An acute lymphoblastic leukemia-associated t(4;11)(q21;q23) translocation involving this gene has also been identified. FLI1 recognizes the DNA sequence 5-C[CA]GGAAGT-3. Human FLI1 protein has 452 amino acids and a molecular mass of 50982 Da.

The term “FLI1” is intended to include fragments, variants (e.g., allelic variants), and derivatives thereof. Representative human FLI1 cDNA and human FLI1 protein sequences are well-known in the art and are publicly available from the National Center for Biotechnology Information (NCBI). Human FLI1 isoforms include the longest isoform 1 (NM_002017.4 and NP_002008.2), and the shorter isoforms 2 (NM_001167681.2 and NP_001161153.1; which contains a distinct 5′ UTR and uses a downstream in-frame start codon, resulting in an isoform with a shorter N-terminus compared to isoform 1), 3 (NM_001271010.1 and NP_001257939.1; which contains a distinct 5′ UTR and 5′ coding region, and uses an alternate start codon, resulting an isoform a distinct and shorter N-terminus compared to isoform 1), and 4 (NM_001271012.1 and NP_001257941.1; which lacks a portion of the 5′ coding region and two alternate internal exons, and uses an alternate start codon, resulting in a shorter isoform with a distinct N-terminus compared to isoform 1). Nucleic acid and polypeptide sequences of FLI1 orthologs in organisms other than humans are well-known and include, for example, chimpanzee FLI1 (1.XM_016922259.1 and XP_016777748.1, 2.XM_016922257.1 and XP_016777746.1, 3.XM_016922258.1 and XP_016777747.1, and 4.XM_016922261.1 and XP_016777750.1), monkey FLI1 (1.XM_015116090.1 and XP_014971576.1, and 2.XM_015116091.1 and XP_014971577.1), dog FLI1 (1.XM_005619564.2 and XP_00561962.1.1, 2.XM_005619566.2 and XP_005619623.1, 3.XM_546404.5 and XP_546404.2), cattle FLI1 (1.NM_001046298.1 and NP_001039763.1), and rat FLI1 (1.NM_001017381.1 and NP_001017381.1).

Anti-FLI1 antibodies suitable for detecting FLI1 protein are well-known in the art and include, for example, antibody AF6474 (R&D systems), antibody TA353818 (OriGene), antibodies AF6474 and NB600-537 (Novus Biologicals, Littleton, Colo.), etc. In addition, reagents are well-known for detecting FLI1 expression. Moreover, multiple siRNA, shRNA, CRISPR constructs for reducing FLI1 expression can be found in the commercial product lists of the above-referenced companies, such as shRNA product # TL312973, siRNA product # SR301621 and CRISPR products # KN200695 from Origene Technologies (Rockville, Md.). It is to be noted that the term can further be used to refer to any combination of features described herein regarding FLI1 molecules. For example, any combination of sequence composition, percentage identify, sequence length, domain structure, functional activity, etc. can be used to describe an FLI1 molecule of the present invention.

The FET-ETS fusion protein is formed by chromosomal translocation, which results in a fusion of the N-terminal of the FET family of RNA binding proteins (e.g., FUS, EWSR1, and TAF15) with the C-terminal of the ETS family of transcription factors (e.g., FLI1, ERG, ETV1, and ETS1). Many of these function as oncoproteins which play important roles in tumorgenesis. For example, the FUS-ERG fusion protein is involved in the development of both acute lymphoblastic leukemia and acute myeloid leukemia. Another example is EWS-FLI1 fusion protein. EWS-FLI1 is a chimeric protein formed by a tumor-specific 11;22 translocation found in both Ewing's sarcoma and primitive neuroectodermal tumor (PNET). Primitive neuroectodermal tumors (PNET) occur either in the central nervous system (CNS; central PNET, cPNET) or in the peripheral sites (peripheral PNET, pPNET). The expression of the genetic rearrangement of EWS-FLI1 is considered to be highly specific to the pPNET and Ewing's sarcoma. Different types of EWS-FLI1 fusions have been identified in these tumors, including Type 1 (SEQ ID No: 1), Type 2 (SEQ ID No: 2), and a novel EWS-FLI1 in-frame isoform fusing EWS to exon 8 of FLI1 isolated from a tumor with a variant t(12;22;22)(q14;p1;q12) translocation (Giovannini M et al., J Clin Invest. 1994 August; 94(2): 489-496). The EWS-FLI1 fusion protein is an aberrant transcription factor that is essential for Ewing tumor development, since it regulates the expression of multiple target genes and governs the oncogenic processes that lead to malignant transformation of a yet undefined cancer precursor cell.

The term “a prion-like domain (PrLD)” refers to a low-complexity domain that possesses a similar amino acid composition to a prion domain in yeast, which enables several proteins, including Sup35 and Rnq1, to form infectious conformers, termed prions. In humans, PrLDs contribute to RNA-binding protein (RBP) function and enable RBPs to undergo liquid-liquid phase transitions that underlie the biogenesis of various membraneless organelles. However, this activity appears to render RBPs prone to misfolding and aggregation connected to neurodegenerative disease. Numerous RBPs with PrLDs, including TDP-43 (transactivation response element DNA-binding protein 43), FUS (fused in sarcoma), TAF15 (TATA-binding protein-associated factor 15), EWSR1 (Ewing sarcoma breakpoint region 1), and heterogeneous nuclear ribonucleoproteins A1 and A2 (hnRNPA1 and hnRNPA2), have now been connected via pathology and genetics to the etiology of several neurodegenerative diseases, including amyotrophic lateral sclerosis, frontotemporal dementia, and multisystem proteinopathy.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Pubis. WO 08/020079, WO 94/02610, WO 95/22618, and WO 03/014960; U.S. Pat. No. 7,004,940; Cattaneo and Biocca (1997) Intracellular Antihodies: Development and Applications (Landes and Springer-Verlag pubis.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A., 90; 6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

The term “biomarker” refers to a measurable entity of the present invention that has been determined to be predictive of cancer therapy effects (e.g., EWS-FLI1 target genes described described herein, such as those in the tables, figures, examples, and otherwise described in the specification). Biomarkers can include, without limitation, nucleic acids (e.g., genomic nucleic acids and/or transcribed nucleic acids) and proteins. Many biomarkers are also useful as therapeutic targets.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of EWS-FLI1 target genes, such as NKX2-2, NPY1R, PPP1R1A, KIT, LOXHD1, MAFB, and NGFR.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated

In certain embodiments, the cancer encompasses Ewing's sarcoma, or Ewing sarcoma. Ewing's sarcoma (EWS) usually occurs in bone and the most common sites for the primary lesion are the pelvic bones, femur, humerus, and ribs. Ewing's sarcoma occurs less commonly at non-bone primary sites, a presentation that has historically been termed extraosseous Ewing's sarcoma. However, the morphological and biological characteristics of Ewing's tumors developing in soft tissues appear to be indistinguishable from those of tumors developing at bone sites. Delattre et al., 1994, New Engl. J. Med. 331:294-299; Llombart-Bosch et al., 1990, Cancer 66:2589-2601. Ewing's sarcoma is more common in males (1.6 male:1 female) and usually presents in childhood or early adulthood, with a peak between 10 and 20 years of age. Most cases of Ewing's sarcoma are the result of a translocation between chromosomes 11 and 22, which fuses the EWSR1 gene of chromosome 22 to the FLI1 gene of chromosome 11 to generate the aberrant transcription factor EWS-FLI11. Other translocations are at t(21;22) and t(7;22).

The diagnosis of Ewing's sarcoma is based on histomorphologic findings, immunohistochemistry and molecular pathology. Ewing's sarcoma is a small-blue-round-cell tumor that typically has a clear cytoplasm on H&E staining, due to glycogen. The presence of the glycogen can be demonstrated with positive PAS staining and negative PAS diastase staining. The characteristic immunostain is CD99, which diffusely marks the cell membrane. Morphologic and immunohistochemical findings are corroborated with an associated chromosomal translocation.

Surgery of Ewing's sarcoma is usually limited to the initial diagnostic biopsy of the primary tumor. Patients usually underwent induction chemotherapy followed by radiation therapy for local control. The successful treatment of patients with Ewing's sarcoma requires the use of multidrug chemotherapy. Combination chemotherapy for Ewing's sarcoma has traditionally included vincristine, doxorubicin, cyclophosphamide, and dactinomycin (VAdriaC or VAC). The importance of doxorubicin has been demonstrated in randomized comparative trials with increased doxorubicin dose intensity during the early months of therapy resulting in improved event-free survival. See, e.g., Nesbit et al., 1990, J. Clin. Oncol. 8:1664-1674; Kinsella et al., 1991, Int. J. Radiat. Oncol. Biol. Phy. 20:389-395; Smith et al., 1991, J. Natl. Cancer Inst. 83:1160 1170.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell,

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. The signal sequence of PD-LS is from about amino acid 1 to about amino acid 18. The signal sequence of PD-L1M is from about amino acid 1 to about amino acid 18. The IgV domain of PD-L1 S is from about amino acid 19 to about amino acid 134 and the IgV domain of PD-L1M is from about amino acid 19 to about amino acid 134. The IgC domain of PD-L1S is from about amino acid 135 to about amino acid 227 and the IgC domain of PD-LM is from about amino acid 135 to about amino acid 227. The hydrophilic tail of the PD-L1 exemplified in PD-L1S comprises a hydrophilic tail shown from about amino acid 228 to about amino acid 245. The PD-L1 polypeptide of PD-L1M comprises a transmembrane domain from about amino acids 239 to about amino acid 259 of PD-L1M and a cytoplasmic domain shown from about amino acid 260 to about amino acid 290 of PD-L1M. In addition, nucleic acid and polypeptide sequences of PD-L1 orthologs in organisms other than humans are well-known and include, for example, rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 of PD-L2 comprises a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Ncurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two ß sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g., T lymphocytes, and 4) modulate the immune response of an organism, e.g., a human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes decreasing, reducing, limiting, and/or blocking, of, for example a particular action, function, and/or interaction. In some embodiments, the interation between two molecules is “inhibited” if the interaction is reduced, blocked, disrupted or destablized.

In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to an agent that inhibits binding of BAF complexes to a FET-ETS fusion protein or FET-ETS-bound GGAA repeat enhancer alone or in combination with an immunotherapy and/or cancer therapy. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to an agent that inhibits binding of BAF complexes to a FET-ETS fusion protein or FET-ETS-bound GGAA repeat enhancer alone or in combination with an immunotherapy and/or cancer therapy, or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

The term “sample” used for detecting or determining the presence or level of at least one biomarker is typically whole blood, plasma, serum, saliva, urine, stool (e.g., feces), tears, and any other bodily fluid (e.g., as described above under the definition of“body fluids”), or a tissue sample (e.g., biopsy) such as bone marrow and bone sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more cancer agents (e.g., an agent that inhibits binding of BAF complexes to a FET-ETS fusion protein or FET-ETS-bound GGAA repeat enhancer in combination with immunotherapy) can be greater than the sum of the separate effects of the cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or oven 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | CAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention are well-known in the art and readily available on publicly available databases, such as the National Center for Biotechnology Information (NCBI). In addition, nucleic acid and amino acid sequence information for the EWS-FLI1 fusion proteins and EWS-FLI1 mutants of the present invention are provided below.

TABLE 1
SEQ ID NO: 1 Human EWS-FLI1 fusion protein type 1 Amino Acid Sequence
MASTDYSTYSQAAAQQGYSAYTAQPTQGYAQTTQAYGQQSYGTYGQPTDVSYTQAQTTATYGQTAYATSYGQP
PTGYTTPTAPQAYSQPVQGYGTGAYDTTTATVTTTQASYAAQSAYGTQPAYPAYGQQPAATAPTRPQDGNKPT
ETSQPQSSTGGYNQPSLGYGQSNYSYPQVPGSYPMQPVTAPPSYPPTSYSSTQPTSYDQSSYSQQNTYGQPSS
YGQQSSYGQQSSYGQQPPTSYPPQTGSYSQAPSQYSQQSSSYGQQNPSYDSVRRGAWGNNMNSGLNKSPPLGG
AQTISKNTEQRPQPDPYQILGPTSSRLANPGSGQIQLWQFLLELLSDSANASCITWEGTNGEFKMTDPDEVAR
RWGERKSKPNMNYDKLSRALRYYYDKNIMTKVHGKRYAYKFDFHGIAQALQPHPTESSMYKYPSDISYMPSYH
AHQQKVNFVPPHPSSMPVTSSSFFGAASQYWTSPTGGIYPNPNVPRHPNTHVPSHLGSYY
SEQ ID NO: 2 Human EWS-FLI1 fusion protein type 2 Amino Acid Sequence
MASTDYSTYSQAAAQQGYSAYTAQPTQGYAQTTQAYGQQSYGTYGQPTDVSYTQAQTTATYGQTAYATSYGQP
PTGYTTPTAPQAYSQPVQGYGTGAYDTTTATVTTTQASYAAQSAYGTQPAYPAYGQQPAATAPTRPQDGNKPT
ETSQPQSSTGGYNQPSLGYGQSNYSYPQVPGSYPMQPVTAPPSYPPTSYSSTQPTSYDQSSYSQQNTYGQPSS
YGQQSSYGQQSSYGQQPPTSYPPQTGSYSQAPSQYSQQSSSYGQQSSLLAYNTTSHTDQSSRLSVKEDPSYDS
VRRGAWGNNMNSGLNKSPPLGGAQTISKNTEQRPQPDPYQILGPTSSRLANPGSGQIQLWQFLLELLSDSANA
SCITWEGTNGEFKMTDPDEVARRWGERKSKPNMNYDKLSRALRYYYDKNIMTKVHGKRYAYKFDFHGIAQALQ
PHPTESSMYKYPSDISYMPSYHAHQQKVNFVPPHPSSMPVTSSSFFGAASQYWTSPTGGIYPNPNVPRHPNTH
VPSHLGSYY
SEQ ID NO: 3 Human EWSR1 N-terminal Prion-like domain Amino Acid Sequence
MASTDYSTYSQAAAQQGYSAYTAQPTQGYAQTTQAYGQQSYGTYGQPTDVSYTQAQTTATYGQTAYATSYGQP
PTGYTTPTAPQAYSQPVQGYGTGAYDTTTATVTTTQASYAAQSAYGTQPAYPAYGQQPAATAPTRPQDGNKPT
ETSQPQSSTGGYNQPSLGYGQSNYSYPQVPGSYPMQPVTAPPSYPPTSYSSTQPTSYDQSSYSQQNTYGQPSS
YGOOSSYGQQSSYGQQPPTSYPPQTGSYSQAPSQYSQQSSSYGQQ
SEQ ID NO: 4 Human FLI1 C-terminal Amino Acid Sequence
PSYDSVRRG AWGNNMNSGL NKSPPLGGAQ TISKNTEQRP QPDPYQILGP TSSRLANPGS
GQIQLWQFLL ELLSDSANAS CITWEGTNGE FKMTDPDEVA RRWGERKSKP NMNYDKLSRA
LRYYYDKNIM TKVHGKRYAY KFDFHGIAQA LQPHPTESSM YKYPSDISYM PSYHAHQQKV
NFVPPHPSSM PVTSSSFFGA ASQYWTSPTG GIYPNPNVPR HPNTHVPSHL GSYY
SEQ ID NO: 5 Human EWS(YS12)-FLI1 Amino Acid Sequence
MASTDYSTYSQAAAQQGSSAYTAQPTQGYAQTTQAYGQQSSGTYGQPTDVSYTQAQTTATYGQTAYATSSGQP
PTGYTTPTAPQAYSQPVQGSGTGAYDTTTATVTTTQASYAAQSAYGTQPAYPAYGQQPAATAPTRPQDGNKPT
ETSQPQSSTGGYNQPSLGSGQSNYSYPQVPGSYPMQPVTAPPSYPPTSSSSTQPTSYDQSSSSQQNTYGQPSS
SGQQSSSGQQSSSGQQPPTSYPPQTGSSSQAPSQYSQQSSSSGQQNPSYDSVRRGAWGNNMNSGLNKSPPLGG
AQTISKNTEQRPQPDPYQILGPTSSRLANPGSGQIQLWQFLLELLSDSANASCITWEGTNGEFKMTDPDEVAR
RWGERKSKPNMNYDKLSRALRYYYDKNIMTKVHGKRYAYKFDFHGIAQALQPHPTESSMYKYPSDISYMPSYH
AHOOKVNFVPPHPSSMPVTSSSFFGAASQYWTSPTGGIYPNPNVPRHPNTHVPSHLGSYY
SEQ ID NO: 6 Human EWS(YS37)-FLI1 Amino Acid Sequence
MASTDSSTSSQAAAQQGSSASTAQPTQGSAQTTQASGQQSSGTSGQPTDVSSTQAQTTATSGQTASATSSGQP
PTGSTTPTAPQASSQPVQGSGTGASDTTTATVTTTQASSAAQSASGTQPASPASGQQPAATAPTRPQDGNKPT
ETSQPQSSTGGSNQPSLGSGQSNSSSPQVPGSSPMQPVTAPPSSPPTSSSSTQPTSSDQSSSSQQNTSGQPSS
SGQQSSSGQQSSSGQQPPTSSPPQTGSSSQAPSQSSQQSSSSGQQNPSYDSVRRGAWGNNMNSGLNKSPPLGG
AQTISKNTEQRPQPDPYQILGPTSSRLANPGSGQIQLWQFLLELLSDSANASCITWEGTNGEFKMTDPDEVAR
RWGERKSKPNMNYDKLSRALRYYYDKNIMTKVHGKRYAYKFDFHGIAQALQPHPTESSMYKYPSDISYMPSYH
AHQQKVNFVPPHPSSMPVTSSSFFGAASQYWTSPTGGIYPNPNVPRHPNTHVPSHLGSYY
SEQ ID NO: 7 Human SYGQ1-FLI1 Amino Acid Sequence
YGQQSYGTYGQPTDVSYTQAQTTATYGQTAYATSYGQNPSYDSVRRGAWGNNMNSGLNKSPPLGGAQTISKNT
EQRPQPDPYQILGPTSSRLANPGSGQIQLWQFLLELLSDSANASCITWEGTNGEFKMTDPDEVARRWGERKSK
PNMNYDKLSRALRYYYDKNIMTKVHGKRYAYKFDFHGIAQALQPHPTESSMYKYPSDISYMPSYHAHQQKVNF
VPPHPSSMPVTSSSFFGAASQYWTSPTGGIYPNPNVPRHPNTHVPSHLGSYY
SEQ ID NO: 8 Human SYGQ2-FLI1 Amino Acid Sequence
TSYDQSSYSQQNTYGQPSSYGQQSSYGQQSSYGQQPPTSYPPQTGSYSQAPSQYSQQSSSYGQQNPSYDSVRR
GAWGNNMNSGLNKSPPLGGAQTISKNTEQRPQPDPYQILGPTSSRLANPGSGQIQLWQFLLELLSDSANASCI
TWEGTNGEFKMTDPDEVARRWGERKSKPNMNYDKLSRALRYYYDKNIMTKVHGKRYAYKFDFHGIAQALQPHP
TESSMYKYPSDISYMPSYHAHQQKVNFVPPHPSSMPVTSSSFFGAASQYWTSPTGGIYPNPNVPRHPNTHVPS
HLGSYY
SEQ ID NO: 9 Human BAF47-FLI1 fusion protein Amino Acid Sequence
MMMMALSKTFGQKPVKFQLEDDGEFYMIGSEVGNYLRMFRGSLYKRYPSLWRRLATVEERKKIVASSHDHGYT
TLATSVTLLKASEVEEILDGNDEKYKAVSISTEPPTYLREQKAKRNSQWVPTLPNSSHHLDAVPCSTTINRNR
MGRDKKRTFPLCFDDHDPAVIHENASQPEVLVPIRLDMEIDGQKLRDAFTWNMNEKLMTPEMFSEILCDDLDL
NPLTFVPAIASAIRQQIESYPTDSILEDQSDQRVIIKLNIHVGNISLVDQFEWDMSEKENSPEKFALKLCSEL
GLGGEFVTTIAYSIRGQLSWHQKTYAFSENPLPTVEIAIRNTGDADQWCPLLETLTDAEMEKKIRDQDRNTRR
MRRLANTAPAWGKPIPNPLLGLDSTPSYDSVRRGAWGNNMNSGLNKSPPLGGAQTISKNTEQRPQPDPYQILG
PTSSRLANPGSGQIQLWQFLLELLSDSANASCITWEGTNGEFKMTDPDEVARRWGERKSKPNMNYDKLSRALR
YYYDKNIMTKVHGKRYAYKFDFHGIAQALQPHPTESSMYKYPSDISYMPSYHAHQQKVNFVPPHPSSMPVTSS
SFFGAASQYWTSPTGGIYPNPNVPRHPNTHVPSHLGSYY
SEO ID NO: 10 Human EWS-FLI1 fusion protein type 1 Nucleic Acid Sequence
atggcgtccacggattacagtacctatagccaagctgcagcgcagcagggctacagtgcttacaccgcccagc
ccactcaaggatatgcacagaccacccaggcatatgggcaacaaagctatggaacctatggacagcccactga
tgtcagctatacccaggctcagaccactgcaacctatgggcagaccgcctatgcaacttcttatggacagcct
cccactggttatactactccaactgccccccaggcatacagccagcctgtccaggggtatggcactggtgctt
atgataccaccactgctacagtcaccaccacccaggcctcctatgcagctcagtctgcatatggcactcagcc
tgcttatccagcctatgggcagcagccagcagccactgcacctacaagaccgcaggatggaaacaagcccact
gagactagtcaacctcaatctagcacagggggttacaaccagcccagcctaggatatggacagagtaactaca
gttatccccaggtacctgggagctaccccatgcagccagtcactgcacctccatcctaccctcctaccagcta
ttcctctacacagccgactagttatgatcagagcagttactctcagcagaacacctatgggcaaccgagcagc
tatggacagcagagtagctatggtcaacaaagcagctatgggcagcagcctcccactagttacccaccccaaa
ctggatcctacagccaagctccaagtcaatatagccaacagagcagcagctacgggcagcagaacccttctta
tgactcagtcagaagaggagcttggggcaataacatgaattctggcctcaacaaaagtcctccccttggaggg
gcacaaacgatcagtaagaatacagagcaacggccccagccagatccgtatcagatcctgggcccgaccagca
gtcgcctagccaaccctggaagcgggcagatccagctgtggcaattcctcctggagctgctctccgacagcgc
caacgccagctgtatcacctgggaggggaccaacggggagttcaaaatgacggaccccgatgaggtggccagg
cgctggggcgagcggaaaagcaagcccaacatgaattacgacaagctgagccgggccctccgttattactatg
ataaaaacattatgaccaaagtgcacggcaaaagatatgcttacaaatttgacttccacggcattgcccaggc
tctgcagccacatccgaccgagtcgtccatgtacaagtacccttctgacatctcctacatgccttcctaccat
gcccaccagcagaaggtgaactttgtccctccccatccatcctccatgcctgtcacttcctccagcttctttg
gagccgcatcacaatactggacctcccccacggggggaatctaccccaaccccaacgtcccccgccatcctaa
cacccacgtgccttcacacttaggcagctactactaa
SEQ ID NO: 11 Human EWS-FLI1 fusion protein type 2 Nucleic Acid Sequence
atggcgtccacggattacagtacctatagccaagctgcagcgcagcagggctacagtgcttacaccgcccagc
ccactcaaggatatgcacagaccacccaggcatatgggcaacaaagctatggaacctatggacagcccactga
tgtcagctatacccaggctcagaccactgcaacctatgggcagaccgcctatgcaacttcttatggacagcct
cccactggttatactactccaactgccccccaggcatacagccagcctgtccaggggtatggcactggtgctt
atgataccaccactgctacagtcaccaccacccaggcctcctatgcagctcagtctgcatatggcactcagcc
tgcttatccagcctatgggcagcagccagcagccactgcacctacaagaccgcaggatggaaacaagcccact
gagactagtcaacctcaatctagcacagggggttacaaccagcccagcctaggatatggacagagtaactaca
gttatccccaggtacctgggagctaccccatgcagccagtcactgcacctccatcctaccctcctaccagcta
ttcctctacacagccgactagttatgatcagagcagttactctcagcagaacacctatgggcaaccgagcagc
tatggacagcagagtagctatggtcaacaaagcagctatgggcagcagcctcccactagttacccaccccaaa
ctggatcctacagccaagctccaagtcaatatagccaacagagcagcagctacgggcagcagagttcactgct
ggcctataatacaacctccCacaccgaccaatcctcacgattgagtgtcaaagaagacccttcttatgactca
gtcagaagaggagcttggggcaataacatgaattctggcctcaacaaaagtcctccccttggaggggcacaaa
cgatcagtaagaatacagagcaacggccccagccagatccgtatcagatcctgggcccgaccagcagtcgcct
agccaaccctggaagcgggcagatccagctgtggcaattcctcctggagctgctctccgacagcgccaacgcc
agctgtatcacctgggaggggaccaacggggagttcaaaatgacggaccccgatgaggtggccaggcgctggg
gcgagcggaaaagcaagcccaacatgaattacgacaagctgagccgggccctccgttattactatgataaaaa
cattatgaccaaagtgcacggcaaaagatatgcttacaaatttgacttccacggcattgcccaggctctgcag
ccacatccgaccgagtcgtccatgtacaagtacccttctgacatctcctacatgccttcctaccatgcccacc
agcagaaggtgaactttgtccctccccatccatcctccatgcctgtcacttcctccagcttctttggagccgc
atcacaatactggacctcccccacggggggaatctaccccaaccccaacgtcccccgccatcctaacacccac
gtgccttcacacttaggcagctactactag
SEQ ID NO: 12 Human FLI1 C-terminal Nucleic Acid Sequence
ccttcttatgactcagtcagaagaggagcttggggcaataacatgaattctggcctcaacaaaagtcctcccc
ttggaggggcacaaacgatcagtaagaatacagagcaacggccccagccagatccgtatcagatcctgggccc
gaccagcagtcgcctagccaaccctggaagcgggcagatccagctgtggcaattcctcctggagctgctctcc
gacagcgccaacgccagctgtatcacctgggaggggaccaacggggagttcaaaatgacggaccccgatgagg
tggccaggcgctggggcgagcggaaaagcaagcccaacatgaattacgacaagctgagccgggccctccgtta
ttactatgataaaaacattatgaccaaagtgcacggcaaaagatatgcttacaaatttgacttccacggcatt
gcccaggctctgcagccacatccgaccgagtcgtccatgtacaagtacccttctgacatctcctacatgcctt
cctaccatgcccaccagcagaaggtgaactttgtccctccccatccatcctccatgcctgtcacttcctccag
cttctttggagccgcatcacaatactggacctcccccacggggggaatctaccccaaccccaacgtcccccgc
catcctaacacccacgtgccttcacacttaggcagctactactag
SEQ ID NO: 13 Human EWS(YS12)-FLI1 Nucleic Acid Sequence
atggcgtccacggattacagtacctatagccaagctgcagcgcagcagggctccagtgcttacaccgcccagc
ccactcaaggatatgctcaaactactcaagcctatggtcagcagtcttccggcacctatggccagcctaccga
cgtgagctatacacaggctcaaacaacggcgacttatgggcaaacagcctatgcgacaagttcaggacaaccg
cctaccggatatactacgccgaccgccccacaggcctattcccagcctgtccaaggttctgggactggcgctt
atgacactactaccgccacggtaactaccacccaagcgtcctatgccgcccagagcgcgtatggcacccaacc
tgcgtatccggcatatggacaacaacccgcagccaccgcccccacgcgccctcaggatggcaataaacccacg
gaaacatcccaaccccaatcatccacgggcggatacaaccagccatcactcggctcagggcaaagtaattaca
gctatcctcaagtgccgggttcctacccgatgcaacccgtcactgcaccgccgtcttacccgcccactagctc
ttcctctacacagccgactagttatgatcagagcagttcctctcagcagaacacctatgggcaaccgagcagc
tctggacagcagagtagctctggtcaacaaagcagctctgggcagcagcctcccactagttacccaccccaaa
ctggatcctccagccaagctccaagtcaatatagccaacagagcagcagctccgggcagcagaacccttctta
tgactcagtcagaagaggagcttggggcaataacatgaattctggcctcaacaaaagtcctccccttggaggg
gcacaaacgatcagtaagaatacagagcaacggccccagccagatccgtatcagatcctgggcccgaccagca
gtcgcctagccaaccctggaagcgggcagatccagctgtggcaattcctcctggagctgctctccgacagcgc
caacgccagctgtatcacctgggaggggaccaacggggagttcaaaatgacggaccccgatgaggtggccagg
cgctggggcgagcggaaaagcaagcccaacatgaattacgacaagctgagccgggccctccgttattactatg
ataaaaacattatgaccaaagtgcacggcaaaagatatgcttacaaatttgacttccacggcattgcccaggc
tctgcagccacatccgaccgagtcgtccatgtacaagtacccttctgacatctcctacatgccttcctaccat
gcccaccagcagaaggtgaactttgtccctccccatccatcctccatgcctgtcacttcctccagcttctttg
gagccgcatcacaatactggacctcccccacggggggaatctaccccaaccccaacgtcccccgccatcctaa
cacccacgtgccttcacacttaggcagctactactaa
SEQ ID NO: 14 Human EWS(Y537)-FLI1 Nucleic Acid Sequence
atggcgtccacggattccagtacctctagccaagctgcagcgcagcagggctccagtgcttccaccgcccagc
ccactcaaggatccgctcaaactactcaagcctccggtcagcagtcttcaggcacctccggccagcctaccga
cgtgagcagtacacaggctcaaacaacggcgactagcgggcaaacagcctccgcgacaagttcaggacaaccg
cctaccggatctactacgccgaccgccccacaggccagttcccagcctgtccaaggttctgggactggcgcta
gtgacactactaccgccacggtaactaccacccaagcgtccagtgccgcccagagcgcgtcaggcacccaacc
tgcgtctccggcaagtggacaacaacccgcagccaccgcccccacgcgccctcaggatggcaataaacccacg
gaaacatcccaaccccaatcatccacgggcggatcaaaccagccatcactcggctcagggcaaagtaattcca
gctcccctcaagtgccgggttcctccccgatgcaacccgtcactgcaccgccgtctagcccgcccactagctc
ttcctctacacagccgactagttctgatcagagcagttcctctcagcagaacacctctgggcaaccgagcagc
tctggacagcagagtagctctggtcaacaaagcagctctgggcagcagcctcccactagttccccaccccaaa
ctggatcctccagccaagctccaagtcaatctagccaacagagcagcagctccgggcagcagaacccttctta
tgactcagtcagaagaggagcttggggcaataacatgaattctggcctcaacaaaagtcctccccttggaggg
gcacaaacgatcagtaagaatacagagcaacggccccagccagatccgtatcagatcctgggcccgaccagca
gtcgcctagccaaccctggaagcgggcagatccagctgtggcaattcctcctggagctgctctccgacagcgc
caacgccagctgtatcacctgggaggggaccaacggggagttcaaaatgacggaccccgatgaggtggccagg
cgctggggcgagcggaaaagcaagcccaacatgaattacgacaagctgagccgggccctccgttattactatg
ataaaaacattatgaccaaagtgcacggcaaaagatatgcttacaaatttgacttccacggcattgcccaggc
tctgcagccacatccgaccgagtcgtccatgtacaagtacccttctgacatctcctacatgccttcctaccat
gcccaccagcagaaggtgaactttgtccctccccatccatcctccatgcctgtcacttcctccagcttctttg
gagccgcatcacaatactggacctcccccacggggggaatctaccccaaccccaacgtcccccgccatcctaa
cacccacgtgccttcacacttaggcagctactactaa
SEQ ID NO: 15 Human SYGQ1-FLI1 Nucleic Acid Sequence
tatgggcaacaaagctatggaacctatggacagcccactgatgtcagctatacccaggctcagaccactgcaa
cctatgggcagaccgcctatgcaacttcttatggacagaacccttcttatgactcagtcagaagaggagcttg
gggcaataacatgaattctggcctcaacaaaagtcctccccttggaggggcacaaacgatcagtaagaataca
gagcaacggccccagccagatccgtatcagatcctgggcccgaccagcagtcgcctagccaaccctggaagcg
ggcagatccagctgtggcaattcctcctggagctgctctccgacagcgccaacgccagctgtatcacctggga
ggggaccaacggggagttcaaaatgacggaccccgatgaggtggccaggcgctggggcgagcggaaaagcaag
cccaacatgaattacgacaagctgagccgggccctccgttattactatgataaaaacattatgaccaaagtgc
acggcaaaagatatgcttacaaatttgacttccacggcattgcccaggctctgcagccacatccgaccgagtc
gtccatgtacaagtacccttctgacatctcctacatgccttcctaccatgcccaccagcagaaggtgaacttt
gtccctccccatccatcctccatgcctgtcacttcctccagcttctttggagccgcatcacaatactggacct
cccccacggggggaatctaccccaaccccaacgtcccccgccatcctaacacccacgtgccttcacacttagg
cagctactactaa
SEQ ID NO: 16 Human SYGQ2-FLI1 Nucleic Acid Sequence
actagttatgatcagagcagttactctcagcagaacacctatgggcaaccgagcagctatggacagcagagta
gctatggtcaacaaagcagctatgggcagcagcctcccactagttacccaccccaaactggatcctacagcca
agctccaagtcaatatagccaacagagcagcagctacgggcagcagaacccttcttatgactcagtcagaaga
ggagcttggggcaataacatgaattctggcctcaacaaaagtcctccccttggaggggcacaaacgatcagta
agaatacagagcaacggccccagccagatccgtatcagatcctgggcccgaccagcagtcgcctagccaaccc
tggaagcgggcagatccagctgtggcaattcctcctggagctgctctccgacagcgccaacgccagctgtatc
acctgggaggggaccaacggggagttcaaaatgacggaccccgatgaggtggccaggcgctggggcgagcgga
aaagcaagcccaacatgaattacgacaagctgagccgggccctccgttattactatgataaaaacattatgac
caaagtgcacggcaaaagatatgcttacaaatttgacttccacggcattgcccaggctctgcagccacatccg
accgagtcgtccatgtacaagtacccttctgacatctcctacatgccttcctaccatgcccaccagcagaagg
tgaactttgtccctccccatccatcctccatgcctgtcacttcctccagcttctttggagccgcatcacaata
ctggacctcccccacggggggaatctaccccaaccccaacgtcccccgccatcctaacacccacgtgccttca
cacttaggcagctactactaa
SEQ ID NO: 17 Human BAF47-FLI1 fusion protein Nucleic Acid Sequence
atgatgatgatggcgctgagcaagaccttcgggcagaagcccgtgaagttccagctggaggacgacggcgagt
tctacatgatcggctccgaggtgggaaactacctccgtatgttccgaggttctctgtacaagagatacccctc
actctggaggcgactagccactgtggaagagaggaagaaaatagttgcatcgtcacatgatcacggatacacg
actctagccaccagtgtgaccctgttaaaagcctcggaagtggaagagattctggatggcaacgatgagaagt
acaaggctgtgtccatcagcacagagccccccacctacctcagggaacagaaggccaagaggaacagccagtg
ggtacccaccctgcccaacagctcccaccacttagatgccgtgccatgctccacaaccatcaacaggaaccgc
atgggccgagacaagaagagaaccttccccctttgctttgatgaccatgacccagctgtgatccatgagaacg
catctcagcccgaggtgctggtccccatccggctggacatggagatcgatgggcagaagctgcgagacgcctt
cacctggaacatgaatgagaagttgatgacgcctgagatgttttcagaaatcctctgtgacgatctggatttg
aacccgctgacgtttgtgccagccatcgcctctgccatcagacagcagatcgagtcctaccccacggacagca
tcctggaggaccagtcagaccagcgcgtcatcatcaagctgaacatccatgtgggaaacatttccctggtgga
ccagtttgagtgggacatgtcagagaaggagaartcaccagagaagtttgccctgaagctgtgctcggagctg
gggttgggcggggagtttgtcaccaccatCgCataCagcatccggggacagctgagctggcatcagaagacct
acgccttcagcgagaaccctctgcccacagtggagattgccatccggaacacgggcgatgcggaccagtggtg
cccactgctggagactctgacagacgctgagatggagaagaagatccgcgaccaggacaggaacacgaggcgg
atgaggcgtcttgccaacacggccccggCctggggtaagcctatccctaaccctctcctcggtctcgattcta
cgccttcttatgactcagtcagaagaggagcttggggcaataacatgaattctggcctcaacaaaagtcctcc
ccttggaggggcacaaacgatcagtaagaataCagagcaacggccccagccagatccgtatcagatcctgggc
ccgaccagcagtcgcctagccaaccctggaagcgggCagatccagctgtggcaattcctcctggagctgctct
ccgacagcgccaacgccagctgtatcacctgggaggggaccaacggggagttcaaaatgacggaccccgatga
ggtggccaggcgctggggcgagcggaaaagcaagcccaacatgaattacgacaagctgagccgggccctccgt
tattactatgataaaaacattatgaccaaagtgcacggcaaaagatatgCttacaaatttgacttccacggca
ttgcccaggctctgcagccacatccgaccgagtcgtccatgtacaagtacccttctgacatctcctacatgcc
ttcctaccatgcccaccagcagaaggtgaactttgtccctccccatccatcctCcatgcctgtcacttcctcc
agcttctttggagccgcatcacaatactggacctcccccacggggggaatctaccccaaccccaacgtccccc
gccatcctaacacccacgtgccttcacacttaggcagctactactag
*Included in Table 1 are RNA nucleic acid molecules (e.g., thymines replaced with
uredines), nucleic acid molecules encoding orthologs of the encoded proteins, as
well as DNA or RNA nucleic acid sequences comprising a nucleic acid sequence having
at least 80%, 81%, 82%,83%, 84%, 85%, 86%, 87%, 88%, 89%, 90%, 91%, 92%, 93%, 94%,
95%, 96%, 97%, 98%, 99%, 99.5%, or more identity across their full length with the
nucleic aicd sequence of any sequence listed in Table 1, or a portion thereof. Such
nucleic acid molecules can have a function of the full-length nucleic acid as
described further herein.
*Included in Table 1 are orthologs of the proteins, as well as polypeptide molecules
comprising an amino acid sequence having at least 80%, 81%, 82%,83%, 84%, 85%, 86%,
87%, 88%, 89%, 90%, 91%, 92%, 93%, 94%, 95%, 96%, 97%, 98%, 99%, 99.5%, or more
identity across their full length with an amino acid sequence of any sequence listed
in Table 1, or a portion thereof. Such polypeptides can have a function of the
full-length polypeptide as described further herein.

### II. Subjects

In one embodiment, the subject for whom an agent that inhibits binding of BAF complexes to a FET-ETS fusion protein or to FET-ETS fusion protein-bound GGAA repeat enhancer elements is administered, or whose predicted likelihood of efficacy of the agent for treating a cancer is determined, is a mammal (e.g., rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to determine the responsiveness to the agent for treating a cancer. In one embodiment, the cancer is Ewing Sarcoma.

### III. Sample Collection, Preparation and Separation

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to cancer therapy (e.g., an agent that inhibits binding of BAF complexes to a FET-ETS fusion protein or to FET-ETS fusion protein-bound GGAA repeat enhancer elements), and/or evaluate a response to a combination cancer therapy (e.g., an agent that inhibits binding of BAF complexes to a FET-ETS fusion protein or to FET-ETS fusion protein-bound GGAA repeat enhancer elements in combination of at least one immunotherapy). A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of cancer therapy. Post-treatment biomarker measurement can be made at any time after initiation of cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of cancer therapy, and even longer toward indefinitely for continued monitoring. Treatment can comprise cancer therapy, such as a therapeutic regimen comprising an agent that inhibits binding of BAF complexes to a FET-ETS fusion protein or to FET-ETS fusion protein-bound GGAA repeat enhancer elements, or in combination with other cancer agents, such as with immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins, Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient, CEC is a hybrid technique between traditional high performance liquid chromatography (HPLC) and CE.

Separation and purification techniques used in the present invention include any chromatography procedures known in the art, Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention. Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an a-anomeric nucleic acid molecule. An a-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak (1993) Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad Sci. USA 93:14670-14675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-3363. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-5988). PNA monomers are then coupled in a step-wise manner to produce a chimeric molecule with a 5′ PNA segment and a 3′ DNA segment (Finn et al. (1996) Nucleic Acids Res. 24:3357-3363). Alternatively, chimeric molecules can be synthesized with a 5′ DNA segment and a 3′ PNA segment (Peterser et al. (1975) Bioorganic Med Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide of the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan (1992) Proc. Natl. Acad. Sci. USA 89:7811-7815; Delgrave et al. 91993) Protein Engineering 6(3):327-331).

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al. (1988) Gene 69:301-315) and pET 11d (Studier et al., p. 60-89, In Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the present invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Camper and Tilghman (1989) Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al (1986) Trendy in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number and/or Genomic Nucleic Acid Mutations

Methods of evaluating the copy number and/or genomic nucleic acid status (e.g., mutations) of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 of a biomarker is predictive of poorer outcome of treatment with the agent inhibiting binding of BAF complexes to a FET-ETS fusion protein or to FET-ETS fusion protein-bound GGAA repeat enhancer elements.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) hound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3; 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-1; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

h. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to a modulator of T cell mediated cytotoxicity alone or in combination with an immunotherapy treatment. Any method known in the art for detecting polypeptides can be used. Such methods include, but are not limited to, immunodiffusion, immunoelectrophoresis, radioimmunoassay (RIA), enzyme-linked immunosorbant assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ²⁵¹I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶ M, 10⁻⁷ M, 10⁻⁸ M, 10⁻⁹ M, 10⁻¹⁰ M, 10⁻¹¹ M, 10⁻¹² M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify the FET-ETS fusion protein/BAF complexes pathway proteins that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J, C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### VI. Cancer Therapies

The efficacy of a cancer therapy with an agent that inhibits binding of BAF complexes to a FET-ETS fusion protein or to FET-ETS fusion protein-bound GGAA repeat enhancer elements is predicted according to biomarker presence, absence, amount and/or activity associated with a cancer in a subject according to the methods described herein. In one embodiment, such cancer therapy (e.g., an agent inhibiting binding of BAF complexes to a FET-ETS fusion protein or to FET-ETS fusion protein-bound GGAA repeat enhancer elements) or combinations of therapies (e.g., an agent inhibiting binding of BAF complexes to a FET-ETS fusion protein or to FET-ETS fusion protein-bound GGAA repeat enhancer elements, in combination with at least one immunotherapy) can be administered to a desired subject or once a subject is indicated as being a likely responder to cancer therapy (e.g., an agent inhibiting binding of BAF complexes to a FET-ETS fusion protein or to FET-ETS fusion protein-bound GGAA repeat enhancer elements). In another embodiment, such cancer therapy (e.g., an agent inhibiting binding of BAF complexes to a FET-ETS fusion protein or to FET-ETS fusion protein-bound GGAA repeat enhancer elements) can be avoided once a subject is indicated as not being a likely responder to the cancer therapy (e.g., an agent inhibiting binding of BAF complexes to a FET-ETS fusion protein or to FET-ETS fusion protein-bound GGAA repeat enhancer elements) and an alternative treatment regimen, such as targeted and/or untargeted cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without the agent inhibiting binding of BAF complexes to a FET-ETS fusion protein or to FET-ETS fusion protein-bound GGAA repeat enhancer elements.

The term “targeted therapy” refers to administration of agents that selectively interact with a chosen biomolecule to thereby treat cancer. One example includes administration of an agent that inhibits binding of BAF complexes to a FET-ETS fusion protein or to FET-ETS fusion protein-bound GGAA repeat enhancers. These agents block or otherwise reduce the interaction between BAF complexes to a FET-ETS fusion protein or to FET-ETS fusion protein-bound GGAA repeat enhancers such that the activation of the FET-ETS fusion protein target genes otherwise induced by the interaction is blocked or otherwise reduced. These agents may inhibit binding of BAF complexes to a FET-ETS fusion protein or to FET-ETS fusion protein-hound GGAA repeat enhancer elements in a direct or indirect way.

Targeted therapy regarding the inhibition of immune checkpoint inhibitor is useful in combination with the methods of the present invention. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). Inhibition of one or more immune checkpoint inhibitors can block or otherwise neutralize inhibitory signaling to thereby upregulate an immune response in order to more efficaciously treat cancer.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Similarly, agents and therapies other than immunotherapy or in combination thereof can be used with in combination with agents inhibiting binding of BAF complexes to a FET-ETS fusion protein or to FET-ETS fusion protein-bound GGAA repeat enhancer elements to treat a cancer that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” referes to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: imercaptopurine and thioguaninc; DNA antimetabolites: 2′-deoxy-5-fluorouridine, aphidicolin glycinatc, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Verloporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Feigner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### VII. Clinical Efficacy

Clinical efficacy can be measured by any method known in the art. For example, the response to a cancer therapy (e.g., an agent inhibiting binding of BAF complexes to a FET-ETS fusion protein or to FET-ETS fusion protein-bound GGAA repeat enhancer elements), relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular agent that inhibits binding of BAF complexes to a FET-ETS fusion protein or FET-ETS fusion protein-bound GGAA repeat enhancer element therapeutic regimen is at least 25%, 30%, 35%, 40%, 45%, 50%, 55%, 60%, 65%, 70%, 75%, 80%, 85%, or more.

Additional criteria for evaluating the response to cancer therapy (e.g., an agent that inhibits binding of BAF complexes to a FET-ETS fusion protein or FET-ETS fusion protein-bound GGAA repeat enhancer element) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent inhibiting binding of BAF complexes to a FET-ETS fusion protein or to FET-ETS fusion protein-bound GGAA repeat enhancer elements can be administered to a population of subjects and the outcome can be correlated to biomarker measurements that were determined prior to administration of any cancer therapy (e.g., an agent inhibiting binding of BAF complexes to a FET-ETS fusion protein or to FET-ETS fusion protein-bound GGAA repeat enhancer elements). The outcome measurement may be pathologic response to therapy given in the neoadjuvant setting. Alternatively, outcome measures, such as overall survival and disease-free survival can be monitored over a period of time for subjects following cancer therapy (e.g., an agent inhibiting binding of BAF complexes to a FET-ETS fusion protein or to FET-ETS fusion protein-bound GGAA repeat enhancer elements) for whom biomarker measurement values are known. In certain embodiments, the same doses of the agent inhibiting binding of BAF complexes to a FET-ETS fusion protein or to FET-ETS fusion protein-bound GGAA repeat enhancer elements are administered to each subject. In related embodiments, the doses administered are standard doses known in the art for the agent inhibiting binding of BAF complexes to a FET-ETS fusion protein or to FET-ETS fusion protein-bound GGAA repeat enhancer elements. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy (e.g., inhibiting binding of BAF complexes to a FET-ETS fusion protein or to FET-ETS fusion protein-bound GGAA repeat enhancer elements) can be determined using methods such as those described in the Examples section.

### VIII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays and xenograft animal model assays. In one embodiment, the assays provide a method for identifying whether a cancer is likely to respond to cancer therapy (e.g., an agent inhibiting binding of BAF complexes to a FET-ETS fusion protein or to FET-ETS fusion protein-bound GGAA repeat enhancer elements), such as in a human by using a xenograft animal model assay, and/or whether an agent can inhibit the growth of or kill a cancer cell that is unlikely to respond to cancer therapy (e.g., an agent inhibiting binding of BAF complexes to a FET-ETS fusion protein or to FET-ETS fusion protein-bound GGAA repeat enhancer elements).

In one embodiment, an assay is a cell-free or cell-based assay, comprising contacting a Ewing sarcoma cancer cell with a test agent, and determining the ability of the test agent to decrease (1) binding of a FET-ETS fusion protein to a BAF complex; (2) binding of a BAF complex to at least one FET-ETS fusion protein-bound GGAA repeat enhancer; and/or (3) expression of at least one a FET-ETS fusion protein target gene.

In another embodiment, an assay is a cell-free or cell-based assay, comprising a) mixing a FET-ETS fusion protein-bound GGAA repeat enhancer element, a FET-ETS fusion protein, and a BAF complex together; b) adding a test agent to the mixture; and c) determining the ability of the test agent to decrease binding of a FET-ETS fusion protein to the BAF complex, and/or binding of the BAF complex to the FET-ETS fusion protein-bound GGAA repeat enhancer element.

For example, in a direct binding assay, one protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product.

Determining the interaction between two molecules (e.g., BAF complexes and a FET-ETS fusion protein) can be accomplished using standard binding or enzymatic analysis assays. These assays may include thermal shift assays (measure of variation of the melting temperature of the protein alone and in the presence of a molecule) (R. Zhang, F. Monsma, Curr. Opin. Drug Discov. Devel., 13 (4) (2010), pp. 389-402), SPR (surface plasmon resonance) (T. Neumann, et al. Curr. Top Med. Chem., 7 (16) (2007), pp. 1630-1642), FRET/BRET (Fluorescence or Bioluminescence Resonance Excitation Transfer) (A. L. Mattheyses, A. I. Marcus, Methods Mol. Biol., 1278 (2015), pp. 329-339; J. Bacart, et al. Biotechnol. J., 3 (3) (2008), pp. 311-324), Elisa (Enzyme-linked immunosorbent assay) (Z. Weng, Q. Zhao, Methods Mol. Biol., 1278 (2015), pp. 341-352), fluorescence polarization (Y. Du, Methods Mol. Biol., 1278 (2015), pp. 529-544), and Far western (U. Mahlknecht, O. G. Ottmann, D. Hoelzer J. Biotechnol., 88 (2) (2001), pp. 89-94) or other techniques. More sophisticated (and lower throughput) biophysical methods that provide structural or thermodynamic details of the molecule binding mode (using isothermal calorimetry (ITC), Nuclear Magnetic Resonance (NMR), and X-ray crystallography) may also be needed for further validation and characterization of potential hits.

Alternatively, high throughput cellular screens measuring the loss of interaction using reverse two hybrid or BRET may be used and offer the advantage of selecting only cell penetrable molecules (A. R. Horswill, S. N. Savinov, S. J. Benkovic Proc. Natl. Acad. Sci. USA, 101 (44) (2004), pp. 15591-15596; A. Hamdi, P. Colas Trends Pharmacol. Sci., 33 (2) (2012), pp. 109-118). The latter approaches require further validation to assess the “on target” effect. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies of the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to inhibit binding of BAF complexes to a FET-ETS fusion protein or FET-ETS fusion protein-bound GGAA repeat enchancers can be accomplished by determining the ability of the test agent to modulate the activity of a polypeptide or other product that functions downstream or upstream of its position within the pathway. For example, it can be accomplished by measuring the activity of the downstream target genes of FET-ETS fusion protein.

The present invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an individual afflicted with a cancer is likely to respond to an agent inhibiting binding of BAF complexes to a FET-ETS fusion protein or to FET-ETS fusion protein-bound GGAA repeat enhancer elements, such as in a cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciate that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to cancer therapy (e.g., an agent that inhibits binding of BAF complexes to a FET-ETS fusion protein or to FET-ETS fusion protein-bound GGAA repeat enhancer elements). In some embodiments, the present invention is useful for classifying a sample (e.g., from a subject) as associated with or at risk for responding to or not responding to cancer therapy (e.g., an agent that inhibits binding of BAF complexes to a FET-ETS fusion protein or to FET-ETS fusion protein-bound GGAA repeat enhancer elements) using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to cancer therapy (e.g., an agent that inhibits binding of BAF complexes to a FET-ETS fusion protein or to FET-ETS fusion protein-bound GGAA repeat enhancer elements) involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely cancer therapy (e.g., an agent that inhibits binding of BAF complexes to a FET-ETS fusion protein or FET-ETS fusion protein-bound GGAA repeat enhancer element) responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptions such as multi-layer perceptions, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to cancer therapy (e.g., an agent that inhibits binding of BAF complexes to a FET-ETS fusion protein or to FET-ETS fusion protein-bound GGAA repeat enhancer elements), a biological sample from the subject during remission, or a biological sample from the subject during treatment for developing a cancer progressing despite cancer therapy (e.g., an agent that inhibits binding of BAF complexes to a FET-ETS fusion protein or to FET-ETS fusion protein-bound GGAA repeat enhancer elements).

d. Prognostic Assays

The diagnostic methods described herein can furthermore be utilized to identify subjects having or at risk of developing a cancer that is likely or unlikely to be responsive to cancer therapy (e.g., an agent that inhibits binding of BAF complexes to a FET-ETS fusion protein or to FET-ETS fusion protein-bound GGAA repeat enhancer elements). The assays described herein, such as the preceding diagnostic assays or the following assays, can be utilized to identify a subject having or at risk of developing a disorder associated with a misregulation of the amount or activity of at least one biomarker described in, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The therapeutic compositions described herein, such as the agent that inhibits binding of BAF complexes to a FET-ETS fusion protein or to FET-ETS fusion protein-bound GGAA repeat enhancer elements, can be used in a variety of in vitro and in vivo therapeutic applications using the formulations and/or combinations described herein. In one embodiment, the therapeutic agents can be used to treat cancers determined to be responsive thereto. For example, single or multiple agents that inhibit binding of BAF complexes to a FET-ETS fusion protein or to FET-ETS fusion protein-bound GGAA repeat enhancer elements can be used to treat cancers in subjects identified as likely responders thereto.

Treatment methods of the present invention involve contacting a cell, such as a cancer cell with an agent that inhibits binding of BAF complexes to a FET-ETS fusion protein or to FET-ETS fusion protein-bound GGAA repeat enhancers. An agent that inhibits binding of BAF complexes to a FET-ETS fusion protein or to FET-ETS fusion protein-bound GGAA repeat enhancer can be an agent as described herein, such as a small molecule, a nucleic acid, a polypeptide, an antibody, or a peptidomimetic. In one embodiment, the agent binds to BAF complexes or the FET-ETS fusion protein at the interaction interface between the BAF complexes and the FET-ETS fusion protein, thereby blocking or competing with the BAF complexes and the FET-ETS fusion protein interaction formation. In another embodiment, the agent binds to another site of the BAF complexes or the FET-ETS fusion protein and capable of inducing a conformational change leading to a loss of interaction with the targeted partner. In yet another embodiment, the agent inhibits the function or activity of a domain or a site of the BAF complexes or the FET-ETS fusion protein that is necessary for the BAF complexes and the FET-ETS fusion protein interaction formation. For example, the agent may inhibit the multimerization and phase transition properties of the N-terminal Prion-like domain of FET protein described herein. In still another embodiment, the agent degrades the BAF complex subunit to which the FET-ETS fusion protein binds, and/or degrades the FET-ETS fusion protein itself, thus breaking the BAF complex and the FET-ETS fusion protein interaction. In one embodiment, the agent is a BAF complex inhibitor. In certain embodiment, the BAF complex inhibitor is Bromosporine, LP99, I-BRD9, BI-9564, BI-7273, GSK-39, dBRD9, or PFI-3. In another embodiment, the agent is a CRISPR/Cas9 reagent that targets the critical residues on the FET-ETS fusion protein or the BAF complexes important for the FET-ETS fusion protein and the BAF complexes interaction, and/or for the multimerization and phase transition properties of the N-terminal Prion-like domain of the FET protein.

These treatment methods can be performed in vitro (e.g., by contacting the cell with the agent) or, alternatively, by contacting an agent with cells in vivo (e.g., by administering the agent to a subject). As such, the present invention provides methods useful for treating an individual afflicted with a condition that would benefit from a decreased activity of FET-ETS target genes by inhibiting binding of BAF complexes to a FET-ETS fusion protein or to FET-ETS fusion protein-bound GGAA repeat enhancer, such as a cancer like Ewing sarcoma. In one embodiment, the method involves administering an agent (e.g., an agent identified by a screening assay described herein), or combination of agents that inhibit FET-ETS target genes expression or activity.

In addition, these inhibitory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### IX. Pharmaceutical Compositions

In another aspect, the present invention provides pharmaceutically acceptable compositions which comprise a therapcutically-cffective amount of an agent that inhibits binding of BAF complexes to a FET-ETS fusion protein or to FET-ETS fusion protein-bound GGAA repeat enhancer elements, formulated together with one or more pharmaceutically acceptable carriers (additives) and/or diluents.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity. These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression. These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a respiration uncoupling agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more respiration uncoupling agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the respiration uncoupling agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

Other embodiments of the present invention are described in the following Examples. The present invention is further illustrated by the following examples which should not be construed as further limiting.

### EXAMPLES

### Example 1: Materials and Methods for Examples 2-8

a. Cell Culture

Mesenchymal stem cells were collected with approval from the Institutional Review Board of the Centre Hospitalier Universitaire Vaudois (CHUV, University of Lausanne). Samples were deidentified prior to our analysis. Primary bone marrow derived mesenchymal stem cells were cultured in Iscove's modified Dulbecco's medium containing 10% fetal calf serum (FCS) and 10 ng/ml platelet-derived growth factor BB (PeproTech), as described previously (Riggi et al., 2010). Cell lines were obtained from ATCC and media from Life Technologies. The Ewing sarcoma cell lines SKNMC and A673 were grown in RPMI. HEK-293-T and U2OS cells were grown in DMEM. All media were supplemented with 10% FBS and cells were cultured at 37° C. with 5% C02. Cells were maintained and split every 2-3 days according to ATCC recommendations. For G1 arrest experiments, A673 cells were treated with Palbociclib (PD 0332991 ISETHIONATE, Sigma-Aldrich) at a final concentration of 1.5 uM for 24 hours.

b. Lentiviral Generation and transfection

Lentivirus was produced in 293T LentiX cells (Clontech) either by polyethylenimine (PEI) (Polysciences Inc.) transfection with gene delivery vector and packaging vectors pspax2 and pMD2.G (Kadoch and Crabtree, 2013) or by LT1 (Mirus Bio) transfection with gene delivery vector and packaging vectors GAG/POL and VSV plasmids (Boulay et al., 2017). Viral supernatants were collected 72 h after transfection and concentrated using either ultracentrifugation (2 hours+4° C. at 20,000 rpm) or LentiX concentrator (Clontech). Virus containing pellets were resuspended in PBS and added dropwise on cells in presence of media supplemented with 6 μg/ml polybrene. Selection of lentivirallyinfected cells was achieved with puromycin used at 0.75-1-2 μg/ml (MSC, SK-N-MC and A673/U20S respectively) and blasticidin used at 7 μg/ml in MSC and U20S. Overexpression or knock-down efficiency was determined by Western blot analysis and RT-qPCR. For transient transfection studies, 293T cells were plated to 80% confluency prior to transfection using LT1 (Mirus Bio) or PEI (Polysciences Inc.) according to the manufacturer recommendations and were collected after 48 h.

c. Real-Time Quantitative RT-PCR

For gene expression assays, total RNA was isolated from cells using NucleoSpin RNA Plus (Clontech). cDNA was obtained using a high-capacity RNA to-cDNA kit (Applied Biosystems). 500 nanograms of template total RNA and random hexamers were used for each reaction. Real-time PCR amplification was performed using fast SYBR Green Master Mix (Life Technologies) and specific PCR primers in a Lightcycler 480 instrument (Roche). Oligonucleotides used are provided in Table 2. Relative quantification of each target, normalized to an endogenous control (GAPDH or HPRT1), was performed using the comparative Ct method (Applied Biosystems). Error bars indicate SD of three technical replicates and represent at least two independent biological experiments. Statistical analyses were performed by Student's t-test. In heatmaps, log 2 qRT-PCR expression values were averaged across biological replicates in each condition and scaled for each gene.

TABLE 2

| | |
|---|---|
| forward | reverse |

RT-qPCR primers

| | | |
|---|---|---|
| KIT | CACCGAAGGAGGCACTTACACA | TGCCATTCACGAGCCTGTCGTA |
| FCGRT | TGGCGATGAGCACCACTACT | GGAGGACTTGGCTGGAGATT |
| CYP4F22 | ATATCCGAGCCGAAGCAGACAC | CGGCATTTCTCCTGGTATTCCG |
| UGT3A2 | TGTTGGAGGCTTGATGGAAAAACC | CGGATTCTGACAGGTGTTCACC |
| CCK | TGAGGGTATCGCAGAGAACGGA | CGGTCACTTATCCTGTGGCTGG |
| NPY1R | CCATCGGACTCTCATAGGTTGTC | GACCTGTACTTATTGTCTCTCATC |
| MAFB | GACGCAGCTCATTCAGCAG | CTCGCACTTGACCTTGTAGGC |
| PPP1R1A | CACAGAAGTGGAGTCAAGGCTG | TTGGCTCCCTTGGAATCCAGTG |
| NR0B1 | AGGGGACCGTGCTCTTTAAC | CTGAGTTCCCCACTGGAGTC |
| NGFR | CCTCATCCCTGTCTATTGCTCC | GTTGGCTCCTTGCTTGTTCTGC |
| FEZF1 | TTCAGCCGAGGCTCTCCTAATG | GCCTGAAACCTTTTCCGCACAC |
| NKX2-2 | CAGCGACAACCCGTACAC | GACTTGGAGCTTGAGTCCTGA |
| PRKCB | GAGGGACACATCAAGATTGCCG | CACCAATCCACGGACTTCCCAT |
| EZH2 | TGGGAAAGTACACGGGGATA | TATTGACCAAGGGCATTCAC |
| GAPDH | GGTCTCCTCTGACTTCAACA | GTGAGGGTCTCTCTCTTCCT |
| HPRT1 | CATTATGCTGAGGATTTGGAAAGG | CTTGAGCACACAGAGGGCTACA |
| LOXHD1 | ACCATCTACGGCGAGGAGTATG | CCCAGGTCAATGGCATAGATGG |
| BAF155 | GAGAATGGACTGAACAGGAGACC | GGGTCCTCAATGGGAAGTCTCA |

ChIP-qPCR primers

| | | |
|---|---|---|
| CCND1 | ACACACCATTTAGTAAAGGCCAA | AGGAATTTGGGGATTTCTCAATCAA |
| SOX2 | GAAGTGCACCCTATGCCAGT | TCCTCTGTGGGGGTTATCCA |
| NR0B1 | GATTCTGTATCAGCTGGTATATACC | GCATCAGGAAGCCTGGATCC |
| LINC00221 | TTCTTTTTGGTCTGCTGGAA | CGTGCCTGACACTCTTCAAC |
| ERRFI1 | AGCCCTTCCAGGAACAGAAC | TTCCCAAATACCCTTCAGCA |
| MYT1 | ATATTGGAGCCCCAAGGGTT | CTCATTAGCAGCGGTGGCAC |

d. Western Blot Analysis

Western blotting was performed using standard protocols. Primary antibodies used for Western blotting are listed in Table 3. Secondary antibodies were goat anti-rabbit and goat anti-mouse immunoglobulin G-horseradish peroxidase-conjugated (Bio-Rad, 1:10,000 dilution). Membranes were developed using Western Lightning Plus-ECL enhanced chemiluminescence substrate (PerkinElmer) and visualized using photographic film. Alternatively, IRDye (Li-COR Biosciences, Lincoln, Nebr., USA) secondary antibodies were used for visualization with the Li-Cor Oddessy Imaging System (Li-COR Biosciences, Lincoln, Nebr., USA).

TABLE 3

| | | | Peptide | | |
|---|---|---|---|---|---|
| | Clone | Type | Region | Source | Catalog # |
| Antibody | | | | | |
| Brg | G7 | mouse monoclonal | aa209-296; | Santa Cruz | sc-17796 |
| | | IgG1 | N-terminus | Biotechnology | |
| Brg | J1 | Rabbit polyclonal | | generated in- | N/A |
| | | | | house | |
| BAF250 | C7 | mouse monoclonal | aa1236-1325 | Santa Cruz | sc-373784 |
| | | IgG1 | | Biotechnology | |
| BAF170 | H-116 | Rabbit polyclonal | aa1093-1208 | Santa Cruz | sc-10757 |
| | | | | Biotechnology | |
| BAF170 | G-12 | mouse monoclonal | C-terminus | Santa Cruz | sc-166237 |
| | | IgG1 | | Biotechnology | |
| BAF155 | | Rabbit polyclonal | aa924-1004; | generated in- | N/A |
| | | | C-terminus | house | |
| BAF155 | H-76 | Rabbit polyclonal | aa978-1073 | Santa Cruz | sc-10756 |
| | | | | Biotechnology | |
| BAF47 | A-5 | mouse monoclonal | aa1-300 | Santa Cruz | sc-166165 |
| | | IgG1 | | Biotechnology | |
| EWS | G-5 | mouse monoclonal | N-Terminus | Santa Cruz | sc-28327 |
| | | IgG1 | | Biotechnology | |
| EWS | | Rabbit polyclonal | aa600; | Bethyl | A300.418A |
| | | | C-terminus | | |
| Fli1 | | Rabbit polyclonal | | abcam | abl5289 |
| BAF60a | 23 | mouse monoclonal | | Santa Cruz | sc-15483 |
| | | IgG1 | | Biotechnology | |
| BRM | | Rabbit polyclonal | aa100-150 | Bethyl | A301.015A |
| GAPDH | FL335 | Rabbit polyclonal | FL (h) | Santa Cruz | sc-25778 |
| | | | | Biotechnology | |
| TBP | | mouse monoclonal | aa1-20 | abcam | ab818 |
| | | IgG1 | | | |
| V5 | | mouse monoclonal | | Abcam | ab27671 |
| LAMIN | | rabbit polyclonal | | Santa Cruz | sc-20681 |
| A/C | | | | | |
| GAPDH | | mouse monoclonal | | Millipore | MAB374 |
| ACTIN | | mouse monoclonal | | Abcam | ab6276 |
| HA | | rat monoclonal | | ROCHE | 11867423001 |
| SS18 | | rabbit polyclonal | | Santa Cruz | sc-28698 |
| GFP | | IgG | | Life | A11122 |
| | | | | Technologies | |
| ChIP | | | | | |
| Antibodies | | | | | |
| Fli1 | | Rabbit polyclonal | | abcam | ab15289 |
| BAF155 | | Rabbit polyclonal | aa924-1004; | generated in- | N/A |
| | | | C-terminus | house | |
| H3K4me1 | | rabbit polyclonal | | abcam | ab8895 |
| H3K27ac | | rabbit polyclonal | | ACTIVE | 39133 |
| | | | | MOTIF | |
| SMARCA2 | | rat monoclonal | | ACTIVE | 39805 |
| | | | | MOTIF | |
| HA | | rat monoclonal | | ROCHE | 11867423001 |

e. Nuclear Extract Preparation

Cells were homogenized in Buffer A (10 mM HEPES (pH 7.6), 25 mM KCL, 1 mM EDTA, 10% glycerol, 1 mM DTT, and protease inhibitors (Roche) supplemented with 1 mM PMSF) on ice. Nuclei were sedimented by centrifugation (1,200 rpm), resuspended in Buffer C (10 mM HEPES (pH 7.6), 3 mM MgCl2, 100 mM KCL, 0.1 mM EDTA, 10% glycerol, 1 mM DTT and protease inhibitors), and lysed by the addition of ammonium sulfate to a final concentration of 0.3M. Soluble nuclear proteins were separated by ultracentrifugation (100,000×g) and precipitated with 0.3 mg/ml ammonium sulfate for 20 mins on ice. Protein precipitate was isolated by ultracentrifugation (100,000×g) and resuspended in IP buffer 1 (300 mM NaCl, 25 mM Hepes [pH 8.0], 0.1% Tween-20, 10% Glycerol, 1 mM DTT, 1 mM PMSF with protease inhibitors) or IP buffer 2 (150 mM NaCl, 50 mM Tris-HCl, 1 mM EDTA, 1% Triton X100, 1 mM DTT, 1 mM PMSF with protease inhibitors) for immunoprecipitation analyses or HEMG-0 buffer (25 mM HEPES [pH 7.9], 0.1 mM EDTA, 12.5 mM MgCl2, 100 mM KCl, supplemented with DTT and PMSF) for analyses on glycerol gradient.

f. Immunoprecipitation

Nuclear extracts were resuspended in IP buffer and placed in protein lo-bind tubes (Eppendorf). Protein concentration was determined using Bradford assay and adjusted to the final volume of 200 μl at a final concentration of 1 mg/ml with IP buffer. Each IP was incubated with 2 μg of antibody (Antibody specifications are found in Table 3) overnight at 4° C. and then for 1 h with 20 μl Protein G Dynabeads (Life Technologies) or Protein G Sepharose beads (GE Heathcare). The beads were then washed five times at 4° C. with IP buffer and resuspended in 20 μl 2×gel loading buffer (4×LDS buffer; Invitrogen)+DTT and water. Alternatively, immunoprecipitations were performed as previously described (Boulay et al., 2017): cells were resuspended in IPH Buffer (50 mM Tris-HCl pH 8, 150 mM NaCl, 5 mM EDTA, 0.5% NP-40 and 10% glycerol supplemented with protease and phosphatase inhibitors (Pierce) and 1 mM PMSF) before sonication in a QSONICA 800 R instrument. Protein supernatant was then collected after centrifugation for 15 min at 14 000 rpm and 4° C. Proteins were quantified using a Bradford assay (Pierce) and 500 μg of lysate were diluted in IPH buffer to a final volume of 1 mL and incubated overnight at +4° C. with 2 μg of the indicated antibodies in the presence of magnetic G-Dynabeads (Life Technologies) and 100 μg/mL Ethidium Bromide (SIGMA-ALDRICH). 100 μg/mL RNase A (Life Technologies) was used for RNase treatment experiments. Beads were washed 5 times with IPH buffer and eluted by boiling in loading Laemmli buffer.

g. Depletion Studies

Nuclear extracts were prepared to a final concentration of 2.5 mg/ml with IP buffer 2. Each IP was incubated with 2.5 μg of antibody overnight at 4° C. and then for 1 h with 15 μl pre-washed Protein G Sepharose beads. After centrifugation (10,000 rpm for Imin) 45 μl of the supernatant was either saved or used for another round of IP. In total 3 rounds of IP were performed. Quantitative densitometry analyses were performed with the Li-Cor Oddessy Imaging System (Li-COR Biosciences, Lincoln, Nebr., USA).

h. Urea Denaturation Studies

Nuclear extracts (150 μg) were subjected to partial urea denaturation, ranging from 0.25 to 2.5 M urea (in IP buffer), for 30 min at room temperature (RT) prior to anti-BRG1 or anti-EWSR1 IP. The co-precipitated proteins were analyzed by immunoblot.

i. Density Sedimentation Analyses

Nuclear extracts (500 μg) were resuspended in 200 ml of 0% glycerol HEMG buffer and carefully overlaid onto a 10 ml 10%-30% glycerol (in HEMG buffer) gradient prepared in a 14 3 89 mm polyallomer centrifuge tube (331327, Beckman Coulter, Brea, Calif., USA). Tubes were centrifuged in an SW40 rotor at 4° C. for 16 hr at 40,000 rpm. Fractions (0.5 ml) were collected and used in analyses.

j. Mass Spectrometry

Immunoprecipitation using either IgG (Cell Signaling Technology) or anti-EWSR1 (Bethyl) crosslinked antibodies was performed in SK-N-MC nuclear extracts. Immunoprecipitation samples were then run on a 4%-12% Bis-Tris Gel (Thermo Scientific) and subjected to Coomassie staining. Bands were then cut from each IP and submitted to the Taplin Biological Mass Spectrometry Facility (Harvard Medical School) for analysis.

k. ChIP-seq and ChIP-qPCR

ChIP assays were carried out on A673, SKNMC and MSCs cultures of approximately 2-5 million cells per sample and per epitope, following the procedures described previously (Ku et al., 2008; Mikkelsen et al., 2007). In brief, chromatin from formaldehyde-fixed cells was fragmented to a size range of 200-700 bases with a Branson 250 sonifier. Solubilized chromatin was immunoprecipitated with the indicated antibodies overnight at 4° C. Antibody-chromatin complexes were pulled down with protein G-Dynabeads (Life Technologies), washed, and then eluted. After crosslink reversal, RNase A, and proteinase K treatment, immunoprecipitated DNA was extracted with AMP Pure beads (Beckman Coulter). ChIP DNA was quantified with Qubit. 1-5 ng ChIP DNA samples were used to prepare sequencing libraries, and ChIP DNA and input controls were sequenced with the Nextseq 500 Illumina genome analyzer.

Reads were aligned to hg19 using bwa (Li and Durbin, 2009). Aligned reads were then filtered to exclude PCR duplicates and were extended to 200 bp to approximate fragment sizes. Density maps were generated by counting the number of fragments, overlapping each position using igvtools, and normalized to 10 million reads. We used macs2 (Zhang et al., 2008) to call peaks using matching input controls with a q-value threshold of 0.01. BAF155 peaks were called using the—broad parameter and were considered promoter peaks if they were within 1 kb of transcriptional start sites (TSSs) in the Refseq database. Peaks were filtered to exclude blacklisted regions as defined by the ENCODE consortium (Consortium, 2012). Peaks within 200 bp of each other were merged. Peak intersections were identified using bedtools (Quinlan and Hall, 2010). Average ChIP-seq signals across intervals were calculated using bwtool (Pohl and Beato, 2014). Total ChIP-seq signals were calculated by multiplying average signals by peak length. For motif discovery and peak annotations, we used Homer suite of tools (Heinz et al., 2010), findMotifsGenome.pl was used to identify de novo motifs within 500 bp flanking centers of distal peaks. The annotatePeaks.pl script was used to assign peaks to annotations. Signals shown in heatmaps (100 bp windows) and composite plots (10 bp window) were calculated using bwtool (Pohl and Beato, 2014). Heatmap signals are in log 2 scale, centered around EWS-FLI1-bound GGAA repeat enhancers previously described (Riggi et al., 2014) (N=812) and are capped at the 99^{th} percentile.

ChIP-qPCR was performed using fast SYBR Green Master Mix (Life Technologies) and specific PCR primers in a Lightcycler 480 instrument (Roche). Oligonucleotides used are provided in Table 2. Relative quantification of each target, normalized to INPUT control, was performed using the comparative Ct method (Applied Biosystems). Error bars indicate SD of three technical replicates and represent at least two independent biological experiments.

1. RNA-Seq

Total RNA was isolated from cells using NucleoSpin RNA Plus (Clontech). 0.5-1 ug of total RNA was treated with Ribogold zero to remove ribosomal RNA. Illumina sequencing libraries were constructed using random primers according to the manufacturer's instructions using the Truseq Stranded RNA LT Kit. Reads were aligned to hg19 using STAR (Dobin et al., 2013). Mapped reads were filtered to exclude PCR duplicates and reads mapping to known ribosomal RNA coordinates, obtained from rmsk table in the UCSC database (http://genome.ucsc.edu). Gene expression was calculated using featureCounts (Liao et al., 2014). Only primary alignments with mapping quality of 10 or more were counted. Counts were then normalized to 1 million reads. Signal tracks were generated using bedtools (Quinlan and Hall, 2010). Differential expression was calculated using DESeq2 (Love et al., 2014). To compare changes in expression of GGAA-repeat associated EWS-FLI1 target genes in MSC experiments, we selected genes based on down regulation upon EWS-FLI1 knockdown in both A673 and SKNMC Ewing sarcoma cells (greater than 2 fold) and a maximum distance of 1 megabase from a EWS-FLI1-bound GGAA repeat (Riggi et al., 2014). Genes were included in heatmaps if they were significantly upregulated (2 fold change and corrected p-value <0.05) in the positive control EWS-FLI1 infection in a given experiment. Normalized expression values were averaged across biological replicates in each condition and scaled for each gene. Principal Component Analysis (PCA) was performed using the R package “prcomp”. Principal components were calculated separately for repeat-associated genes (as described above) and non-repeat-associated genes with reciprocal changes in EWSFLI1 knockdowns (2 fold in A673 and SKNMC cells) and MSCs infected with EWS-FLI1 (2 fold and corrected p-value <0.05). PC rotations obtained by evaluating MSCs infected with EWS-FLI1 and controls were used to calculate PC1 scores for other conditions.

m. ATAC-Seq Genome-Wide DNA Accessibility Assay

ATAC-seq analysis on human pediatric MSCs was performed as recently described (Buenrostro et al., 2013). Briefly, 5×104 cells for each condition were first incubated in hypotonic buffer then resuspended in lysis buffer, centrifugated and resuspended in Transposase reaction mix for additional 30 min at 37° C., following manufacturer recommendations (Nextera DNA sample Prep Kit, Illumina). After DNA purification, adaptor sequences were added to the fragmented DNA by PCR. Purified PCR products were sequenced using the Illumina Nextseq device. Paired end reads were aligned to hg19 using bwa (Li and Durbin, 2009). Reads that aligned in the proper orientation and on the same chromosome were then filtered to exclude PCR duplicates and processed as previously described (Buenrostro et al., 2013). Briefly, read start sites were adjusted to represent the center of the transposon binding event (+4 bp in the plus strand and −5 bp in the minus strand). Signal densities were calculated over a sliding 150 bp window at 20 bp steps and normalized to 10 million reads in each experiment using bedtools (Quinlan and Hall, 2010). Average ATAC-seq signals across intervals was calculated using bwtool (Pohl and Beato, 2014). To test chromatin opening in non-GGAA repeat sites after BAF47-FLI1 expression, ATAC-seq signals were measured at the top 10,000 new peaks detected with the FLI1 antibody after introduction of BAF47-FLI1.

n. Immunofluorescence Staining

Staining was performed using standard protocols. Briefly, cells were fixed in a 1×PBS solution containing 4% paraformaldehyde 15 minutes at room temperature (RT), washed with 1×PBS and stored at 4° C. Cells were permeabilized 10 minutes at RT in 1×PBS containing 0.5% Triton X-100 then blocked for 30 minutes at RT, stained with the relevant antibody for 2 hours at RT and with Alexa-Fluor 546-conjugated secondary antibody (Life Technologies) for 1 h at RT in the dark. Cells were washed with IX PBS between each step of the protocol. Nuclei were stained with DAPI solution.

o. Cell Viability Assays

Cells were seeded in triplicates and grown under log phase growth conditions in cell culture treated 96 well plate. After the indicated incubation times, cell viability was measured using the CellTiter-Glo luminescent assay (Promega) as described by the manufacturer. Endpoint luminescence was measured on a SpectraMax M5 plate reader (Molecular Devices). The data displayed are representative of at least two biological experiments. Statistical analyses were performed by Student's t-test.

p. Biotinylated Isoxazole-Mediated Precipitation

These assays were performed as previously described (Kato et al., 2012) with slight modifications. Biotinylated isoxazole (b-isox, Sigma-Aldrich) was reconstituted in DMSO. Briefly, 5-10 million cells were resuspended in 1 mL lysis buffer (20 mM Tris-HCl pH 7.4, 150 mM NaCl, 5 mM MgCl2, 0.5% NP-40 and 10% glycerol supplemented with 1× Protease/Phosphatase inhibitors (Pierce), 0.1 mM PMSF and 20 mM betamercaptoethanol) and incubated for 30 minutes with rotation at +4° C. Protein supernatant was then collected after centrifugation for 15 min at 14000 rpm+4° C. A 5% whole cell extract (WCE) control was collected and the remaining proteins were divided into four aliquots before addition of biotinylated isoxazole at 0, 10, 30 100 uM final concentrations. 100 μg/mL RNase A (Life Technologies) was used for RNase treatment experiments. The Reaction solutions were incubated at +4° C. for 1 h with rotation and centrifuged for 15 min at 14000 rpm+4° C. Supernatant was saved for further analysis and pellets were washed twice in supplemented Lysis buffer before resuspension in Laemmli buffer. WCE, pellets and supernatants were analyzed by 4%-12% Tris-Glycine gradient gels (Life Technologies) and Western blotting was performed using standard protocols.

q. Protein Purification and Sedimentation Assays

Recombinant proteins were purified as previously described (Couthouis et al., 2011; Sun et al., 2011) with minor modifications. Wild type and mutant constructs were cloned in pGEX-6P1, and expressed in E. coli BL21 (DE3) pLysS (Fisher Scientific). Bacteria were grown in LB medium supplemented with 100 μg/ml ampicillin and 25 μg/ml chloramphenicol. At absorbance A600=0.8, protein expression in E. coli cultures was induced by adding 0.1 mM IPTG and incubated overnight at 16° C. with agitation. Cells were harvested by centrifugation and cell pellets were resuspended in 40 ml GST buffer (1×PBS, 150 mM NaCl, 1.5 mM EDTA, 1 mM DTT, 0.2% Triton X100 and Protease/phosphatase inhibitors (Sigma)). The homogenized suspension was sonicated 3 times on ice for 30 sec. The insoluble materials were removed by centrifugation (15,000 rpm 20 min at 4° C.) and the clarified supernatants were incubated with glutathione sepharose beads (GE Healthcare) or glutathione magnetic agarose beads (Pierce) for 2 h at 4° C. The beads were washed 3 times with GST washing buffer (1×PBS, 350 mM NaCl, 1 mM EDTA, and 1 mM DTT) and once with equilibration buffer (50 mM.Tris-HCl pH 8, 100 mM KAc, 200 mM trehalose, 0.5 mM EDTA). Recombinant proteins were eluted with GST elution buffer (50 mM Tris-HCl pH 8, 100 mM KAc, 200 mM trehalose, 0.5 mM EDTA, 20 mM glutathione) for 2 h at 4° C. Eluted proteins were centrifuged (16,000 g 10 min at 4° C.) prior to quantification by Bradford assay. Recombinant proteins (2.5 μM) were then resuspended in reaction buffer (50 mM Tris.HCl, pH 8, 100 mM potassium acetate, 200 mM trehalose, 0.5 mM EDTA, 20 mM glutathione, 60 U/mL Scission Protease) and incubated at 25° C. for 60 minutes with agitation (1,200 rpm) in an Eppendorf Thermomixer before centrifugation at 16,000 g for 10 minutes at +25° C. Recombinant GST-EWS-FLI1 bound to magnetic beads was quantified against a bovine serum albumin standard dilution by SDS page and Coomassie staining and magnetic beads were removed before final centrifugation. Supernatant and pellet fractions were then resolved by SDS PAGE and stained with Coomassie Blue. The amount in either fraction was determined by densitometry in comparison to an input reaction control using ImageJ software.

r. Accession Numbers

The data accompanying this paper have been deposited into GEO under accession number GSE94278.

TABLE 4

| shRNA | position | |
|---|---|---|
| target | on target | sequence |
| FLI1 | 1917 | CGTCATGTTCTGGTTTGAGAT |
| BAF155 #1 | 727 | CCCACCACATTTACCCATATT |
| BAF155 #2 | 4563 | GCAGGATATTAGCTCCTTATA |
| BRG1 | 5279 | GGCATAGGCCTTAGCAGTAAC |

### Example 2: mSWI/SNF (BAF) Complexes Interact with Wild-Type EWSR1 and the Fusion Protein EWS-FLI1

BAF complexes are combinatorially assembled from a set of ubiquitously expressed core subunits as well as many cell type- and context-specific subunits that give rise to a diversity of supra-molecular configurations. In order to identify the constellation of BAF complex subunits and associated proteins, endogenous capture of BAF complexes was performed via anti-BRG 1 immunoprecipitation followed by proteomic mass-spectrometry in several cell types. Notably, these experiments revealed substantial enrichment of peptides corresponding to the EWSR1 protein, among several other previously unidentified proteins (FIG. 1A). EWSR1 has been linked to several cellular processes but most notably it has been shown to be directly involved in gene regulation as a frequent partner in oncogenic fusion proteins with transcription factors such as the EWS-FLI1 protein in Ewing sarcoma (Mertens et al., 2016). Given the strong connection between BAF and gene regulation in cancer whether EWSR1 as well as the EWS-FLI1 fusion protein can interact with BAF complexes was proceeded to confirm. Immunoprecipitation experiments using antibodies specific for BRG1 performed on nuclear extracts isolated from EWSR1 wild-type cells (SAOS2 and U2OS) or EWS-FLI1-positive Ewing sarcoma cell lines revealed that both EWSR1 and the EWS-FLI1 fusion protein interact with BAF complexes (FIG. 13). Reciprocal immunoprecipitation experiments using an antibody specific for EWSR1 confirmed these interactions (FIG. 2A). Similar experiments using antibodies specific to additional BAF complex subunits, BAF155 and SS18, also confirmed these interactions with EWSFLI1 in Ewing sarcoma (FIG. 2B).

To further characterize the interaction of EWS-FLI1 with BAF complexes, immunodepletion experiments using an anti-BRG1 antibody in SK-N-MC Ewing sarcoma cell nuclear extracts was performed to determine the relative fraction of EWS-FLI1 bound to BAF complexes. EWS-FLI1 was significantly depleted from nuclear lysates over three rounds of anti-BRG1 immunoprecipitation, suggesting that a high percentage of total nuclear EWS-FLI1 can associate with BAF complexes (FIG. 1C). Reciprocal immunodepletion experiments using an EWSR1 antibody were only able to slightly deplete BAF complex components, indicating that only a small percentage of total BAF complexes are bound to EWS-FLI1 and wild-type EWSR1 (FIG. 2C), consistent with genome-wide activities for this chromatin remodeling complex that are independent of these proteins. This was further substantiated by density sedimentation experiments, which showed that neither EWSR1 nor EWS-FLI1 were core members of the BAF complex (FIG. 2D). Similarly, urea denaturation studies showed that EWSR1 interactions with BAF complex subunits were decreased at ˜0.5 M urea (FIG. 2E) and were thus weaker than those observed between core BAF complex members (≥2.5 M urea)(Kadoch and Crabtree, 2013). Taken together, these data demonstrate that both wild-type EWSR1 and the oncogenic fusion protein EWS-FLI1 interact with BAF complexes in a transient manner.

### Example 3: EWS-FLI1 Recruits BAF Complexes to Tumor-Specific GGAA Microsatellite Repeat Enhancers to Activate Target Gene Expression

Owing to the biochemical interaction between BAF complexes and EWS-FLI1, whether BAF complexes cooperate with EWS-FLI1 to regulate gene expression in Ewing sarcoma was next sought to determine. EWS-FLI1 operates as a pioneer factor to induce tumor-specific de novo enhancers at GGAA microsatellite repeats was recently demonstrated (Riggi et al., 2014), a process that may involve significant redistribution of chromatin remodeling complexes which directly govern DNA accessibility such as BAF (Phelan et al., 1999). To address a potential collaborative role in this process, ChIP-seq studies were performed to localize BAF complexes and EWSFLI1 occupancy in the Ewing sarcoma cell line SK-N-MC, using BAF155 and FLI1 antibodies, respectively. The majority (95.4%) of BAF155 MACS-called peaks were detected at putative enhancer regions (FIG. 1D and FIG. 2F). Moreover, a striking degree of overlap between BAF155 and EWS-FLI1 sites genome-wide was detected for which the GGAA repeat was the top-ranked DNA motif (FIG. 1E). In addition to substantial co-localization, median BAF155 peak occupancy was two-fold higher at EWS-FLI1-bound GGAA repeats compared to all other genomic locations (FIG. 1F), and was centered on EWS-FLI1 peaks (FIGS. 1G-I and FIG. 2G), suggesting that the localization of BAF complexes may be dependent on EWS-FLI1 binding. To test this hypothesis, EWS-FLI1 in SK-N-MC Ewing sarcoma cells was depleted using shRNAs and found that suppression of EWS-FLI1 led to an almost complete disappearance of BAF complex occupancy at GGAA repeats (FIGS. 2A-C and FIG. 4A), while other BAF155 peaks outside these GGAA enhancer regions remained unaffected (FIG. 2D). These results were further validated by ChIP-qPCR for BAF155 occupancy over selected loci containing GGAA repeats, including CCND1, SOX2, NR0B1, and LINC00221 (FIG. 2E and FIG. 4B), as well as by using an antibody that recognizes the alternative BAF ATPase core subunits BRG1/BRM (FIG. 4C-E). To account for possible effects of the cell cycle arrest observed after EWS-FLI1 knockdown (Tanaka et al., 1997), A673 cells were arrested in the G1 phase prior to performing ChIP-qPCR (FIG. 4F) and co-immunoprecipitation assays (FIG. 4G). Interactions and recruitment at chromatin were stable in these conditions, further confirming the specificity of decreased BAF complex occupancy at GGAA repeats observed in the absence of EWS-FLI1. These observations thus indicate that the presence of BAF complexes at GGAA repeat microsatellites is dependent on the EWS-FLI1 fusion protein.

To evaluate the potentially instructive nature of EWS-FL1l in targeting BAF complexes, whether EWS-FLI1 is capable of site-specific recruitment of BAF complexes in mesenchymal stem cells (MSCs) was assessed. Stable introduction of EWS-FLI1 into MSCs has been demonstrated to be a suitable model for functional studies as these cells represent a likely cell of origin of Ewing sarcoma (Riggi et al., 2005; Riggi et al., 2008). In this system, EWS-FLI1 has been shown to operate as a pioneer factor at GGAA repeats (Riggi et al., 2014). Whereas in naïve MSCs, BAF complex occupancy over GGAA repeats was undetectable, a substantial increase in BAF complex localization upon introduction of EWS-FLI1 (FIGS. 2F-G) was observed. Notably, GGAA repeats were the highest ranking DNA motif at newly created BAF complex sites, consistent with a major EWS-FLI1-mediated redistribution of BAF complexes (FIG. 2H).

Whether BAF complexes are required for the induction of EWS-FLI1-mediated gene expression was next assessed. ShRNA-mediated depletion of the BRG1 ATPase prior to expression of EWS-FLI1 in MSCs caused a striking reduction in target gene activation (FIG. 2I). Furthermore, knockdown of the subunit BAF155 in SK-N-MC Ewing sarcoma cells, using two independent BAF155-specific shRNAs, resulted in decreased expression of EWS-FLI1 target genes (FIG. 2J and FIG. 4H) and significantly impaired viability of Ewing sarcoma cells in culture (FIG. 4I). A similar decrease in viability was observed using shRNAs targeting the BRG1 ATPase subunit (FIG. 4J). These studies performed in both MSCs and Ewing sarcoma cell lines indicate that BAF complexes play a major role in EWS-FLI1-mediated oncogenic gene regulation.

### Example 4: Recruitment of BAF Complexes to GGAA Repeats is a Neomorphic Property of the EWS-FLI1 Fusion Compared to Wild-Type FLI1

The respective contributions of EWS and FLI1 components of the EWS-FLI1 fusion protein to chromatin reorganization have not to date been fully delineated. As such, whether expression of the FLI1 transcription factor alone could recruit BAF complexes to GGAA repeats was aimed to explore. In contrast to expression of EWS-FLI1, expression of the wild-type FLI1, which provides the ETS DNA binding domain to the fusion protein, did not induce de novo activation of enhancers at GGAA repeats in MSCs and was not associated with recruitment of BAF complexes (FIGS. 3A-B and FIG. 6A). Furthermore, while FLI1 binding was readily detected at non-repeat canonical ETS binding motifs (FIG. 6B-C), significant binding for FLI1 at GGAA repeat sites was not observe (FIGS. 3A-B and FIG. 6D). Accordingly, FLI1 failed to induce the expression of known EWS-FLI1 target genes (FIG. 6E). Tethering to GGAA repeats and the recruitment of BAF complexes to these sites is thus a neomorphic property of the EWSFLI1 fusion protein compared to wild-type FLI1.

In order to test whether these striking differences between EWS-FLI1 and wildtype FLI1 were linked to their differential ability to bind BAF complexes, immunoprecipitation experiments was performed comparing FLI1 and EWS-FLI1. Immunoprecipitation of lentivirally-expressed FLI1 or EWS-FLI1 using an anti-FLI1 antibody showed that both proteins were capable of pulling-down the BAF complex subunit BRG1 (FIG. 3C). Consistent with these observations, further characterization showed that both the EWS N-terminal and FLI1 C-terminal fragments of EWS-FLI1 were able to coimmunoprecipitate BRG1 (FIG. 3D). To test whether an additional interaction domain could account for the neomorphic properties of EWS-FLI1, a fusion protein between the BAF complex subunit BAF47 and the FLI1 C-terminal domain was generated (FIG. 3E). This fusion protein was able to interact with the BAF complex, as demonstrated by immunoprecipitation (FIG. 3F), and had the ability to hind and increase chromatin opening when expressed in MSCs (FIG. 7A-D). However, in contrast to EWS-FLI1, the BAF47-FLI1 fusion protein failed to display significant binding to GGAA repeats (FIG. 7E), to induce de novo enhancer formation (FIG. 3G and FIG. 7F), or to activate target gene expression (FIG. 3H and FIG. 7G). Thus, fusion of the FLI1 C-terminus directly to the BAF chromatin remodeling complex is insufficient to replicate the pioneer function of the EWS-FLI1-bound BAF complexes at GGAA repeats, suggesting additional critical properties of the EWSR1 fragment of the fusion protein.

### Example 5: Fusion of EWSR1 to FLI1 Confers Prion-Like Phase Transition Properties

Phase transition is defined as the ability of a biological system to undergo a change of phase or state, including transitions from protein solutions to liquid-like phase separated compartments that constitute membrane-less organelles (Aguzzi and Altmeyer, 2016). EWSR1 belongs to the FET family of proteins, which also includes both FUS and TAF 15, and is characterized by intrinsically disordered low-complexity prion like domains that have been shown to mediate multimerization, physiological liquid-liquid phase separation and pathological protein aggregation (Couthouis et al., 2012; Couthouis et al., 2011; Kato et al., 2012; Kwon et al., 2013; Schwartz et al., 2013; Thomsen et al., 2013). In keeping with these findings, the multimerization potential of EWSR1 was confirmed by introducing GFP-tagged EWSR1 into U2OS cells and performing anti-GFP immunoprecipitation, which captured both the exogenously introduced variant (GFP-EWSR1) and endogenous EWSR1 (FIG. 9A).

Given that the FET prion-like domains can undergo multimerization and concentration-dependent phase transition (Kato et al., 2012; Patel et al., 2015) and are retained in EWS-FLI1 as well as in other oncogenic fusions between FET proteins and transcription factors (Mertens et al., 2016), whether EWS-FLI1 can multimerize in the setting of Ewing sarcoma cells was first tested. In keeping with previous studies (Embree et al., 2009; Spahn et al., 2003), immunoprecipitation of tagged EWS-FLI1 showed a strong interaction with endogenous EWSR1 (FIG. 8A). In contrast, tagged wild-type FLI1 only produced weak signals in these assays (FIG. 8A). This suggests that a major difference between FLI1 and the fusion protein EWS-FLI1 is the ability to interact with wild-type EWSR1. In further support of this finding, immunoprecipitation followed by mass spectrometry of endogenous EWSR1 in SK-N-MC Ewing sarcoma cells, using an antibody targeting the C-terminus of EWSR1 not present in EWS-FLI1, showed strong reciprocal interactions with EWS-FLI1 and also with other FET family proteins (TAF15 and FUS) as well as several members of the BAF complex (FIG. 8B).

In order to assess the phase transition potential of EWS-FLI1, its ability to precipitate was first tested in presence of biotinylated isoxazole (b-isox), a recently identified compound with the ability to precipitate proteins with low complexity domains including FET family proteins (Han et al., 2012; Kato et al., 2012). These experiments show that EWS-FLI1 exhibits robust, concentration-dependent precipitation, comparable to and even higher than wild-type EWSR1 in SK-N-MC Ewing sarcoma cells (FIG. 8C-D). This observation was recapitulated in U2OS cells stably expressing EWS-FLI1, but not in U2OS cells expressing wild-type FLI1 using the same conditions (FIG. 8E).

Given that the presence of RNA has previously been shown to affect some EWSR1 interactions (Spahn et al., 2003), the effects of RNase treatment in the experiments was tested. RNase reduced b-isox induced precipitation of EWSR1 but had small effects on EWS-FLI1 (FIG. 9B). Similarly, interactions between EWS-FLI1, EWSR1 and BAF complexes (BRG1) were not significantly affected by RNase treatment (FIG. 9C-D). These results suggest distinct properties for EWS-FLI1 containing complexes and match previous in vitro results showing decreased EWSR1 homotypic interactions and stable heterotypic interactions with EWS-FLI1 upon RNase treatment (Spahn et al., 2003).

Wild-type EWSR1 has been previously shown to spontaneously precipitate in sedimentation experiments used to measure the intrinsic phase transition potential of purified proteins in vitro (Couthouis et al., 2012), bacterially expressed GST-tagged EWS-FLI1 and wild-type FLI1 were thus purified to test their intrinsic phase transition potential. Sedimentation assays revealed that most EWS-FLI1 spontaneously precipitated, while wild-type FLI1 remained soluble in these conditions (FIGS. 8F-G and FIG. 9E). These results demonstrate that the EWSR1 prion-like domain confers neomorphic intrinsic phase transition properties to the EWS-FLI1 oncogenic fusion protein. Supporting these observations, confocal imaging showed that EWS-FLI1 was detectable as nuclear dot-like structures after lentiviral expression in MSCs while wildtype LI1 exhibited a more diffuse pattern (FIG. 9F).

Having demonstrated interactions between EWS-FLI1 and EWSR1 and similar phase transition properties conferred by their prion-like domain in vitro, these proteins in the same complexes at GGAA repeats in Ewing sarcoma cells was expected to find. To assess this, HA-tagged EWSR1 was introduced into A673 Ewing sarcoma cells, confirmed its nuclear localization and its ability to interact with endogenous EWS-FLI1 and the BAF complex ATPase subunit BRG 1 (FIGS. 9G-H) and assessed its presence at EWS-FLI1-bound GGAA microsatellite repeats. Importantly, substantial co-enrichment of HA-EWSR1 and EWS-FLI1 at GGAA microsatellite repeats was observed, both by ChIP-seq and by validation ChIP-qPCR over selected target sites (FIGS. 8H-J and FIG. 9I-J). Furthermore, occupancy of HA-EWSR1 was decreased at these loci upon shRNA-mediated suppression of EWS-FLI1 (FIG. 9K). Taken together, these observations suggest that EWS-FLI1 and wild-type EWSR1 are both present in the same macromolecular complexes at GGAA repeats in Ewing sarcoma.

### Example 6: Tyrosine Residues in the EWS-FLI1 Prion-Like Domain are Necessary for DNA Binding at GGAA Micro-Satellites and De Novo Enhancer Activation

whether phase transition mediated by the prion-like domain of EWS-FLI1 is necessary for DNA binding, BAF complex recruitment, and de novo enhancer activation at GGAA microsatellite repeats was next determined by generating a series of V5-tagged EWS-FLI1 mutant proteins lacking the ability to precipitate in vitro. The EWSR1 prion like domain is rich in [G/S]Y[G/S] motifs (FIG. 11A), and the substitution of these tyrosine residues with serine has been shown to abrogate phase transitions to hydrogels observed for the FET protein FUS (Kato et al., 2012; Kwon et al., 2013). Thus, two EWS-FLI1 mutant proteins with point mutations altering either 12 or all 37 tyrosines in the prion-like domain (namely EWS(YS12)-FLI1 and EWS(YS37)-FLI1) were generated (FIG. 10A and FIG. 11A). Both mutants were expressed and localized to the nucleus of lentivirally transduced MSCs as assessed by immunofluorescence in a manner comparable to that of EWS-FLI1 (FIG. 11B-C). The EWS(YS12)-FLI1 mutant protein maintained significant interactions with wild-type EWSR1 and BRG1, and although diminished, b-isox induced precipitation remained significantly higher than wild-type FLI1 (FIGS. 10B-C and 8E). In contrast, EWS(YS37)-FLI1 displayed reduction in binding to wild-type EWSR1 and BRG1 as well as a profound loss of b-isox induced precipitation to levels comparable to those of wild-type FLI1 (FIGS. 10B-C and 8E). EWS(YS37)-FLI1, however, maintained the ability to homodimerize, a hallmark of ETS family transcription factors, as assessed by reciprocal immunoprecipitation experiments using overexpressed HA- and V5-tagged variants in HEK-293-T cells (FIG. 11D). In keeping with the results obtained with b-isox, in vitro sedimentation assays performed with purified GST-tagged EWS(YS37)-FLI1 showed a complete loss of its ability to spontaneously precipitate (FIG. 10D and FIG. 11E).

Based on these findings, the ability of the EWS(YS37)-FLI1 mutant protein to bind GGAA microsatellite repeats and to create active enhancers once expressed in MSCs was further tested (FIG. 11F). ChIP-seq experiments clearly demonstrated a dramatic reduction in binding of the EWS(YS37)-FLI1 mutant at these sites, as well as impaired BAF complex recruitment (FIG. 10E and FIG. 11G). In line with this observation, DNA accessibility and marks of enhancer activity assessed by, respectively, ATAC-seq and ChIP-seq for H3K27ac were undetectable for the EWS(YS37)-FLI1 mutant (FIGS. 10E-G and FIG. 11H-I). Finally, consistent with the impairment of its biochemical properties, EWS(YS37)-FLI1 was not able to induce expression of GGAA microsatellite target genes after introduction in MSCs, while EWS(YS12)-FLI1 retained nearly full activity (FIG. 10H). Taken together, these results demonstrate that the tyrosine residues in the EWS-FLI1 prion-like domain are necessary to mediate phase transitions and are required for its pioneer activity by allowing stable DNA binding, BAF complex recruitment at GGAA repeat microsatellites, and target gene activation.

### Example 7: Fusion of EWSR1 Prion-Like Domain Fragments to the FLI1 C-Terminus is Sufficient to Recapitulate EWS-FLI1 Activity

Given that prion-like domains are unstructured, low-complexity protein sequences, whether any specific region within this domain is critical for EWS-FLI1 function was next tested. To this end, a series of EWS-FLI1 internal deletion mutants was generated (FIG. 13A). While the tyrosine residues that were shown to be critical are mostly evenly distributed over the EWSR1 prion-like domain, there are two regions that contain exact [G/S]Y[G/S] motifs followed by a glutamine (SYGQ), which have been designated as SYGQ1 (also called FETBM1)(Thomsen et al., 2013) and SYGQ2. These regions were thus deleted either independently or in combination (FIG. 13A). All EWS-FLI1 deletion mutant proteins accumulated in the nucleus of MSCs and exhibited comparable binding to wild-type EWSR1 and BRG1 (FIG. 13B-D). Similarly, EWSFLI1 deletion mutants maintained b-isox induced precipitation properties that were diminished when compared to EWS-FLI1 but significantly higher than those of wild-type FLI1 (FIG. 13E). Changes in target gene expression after introduction of the different EWS-FLI1 mutants in MSCs were next assessed and a large set of target genes activated by GGAA microsatellites was still strongly induced by all constructs was observed (FIG. 13F). Thus, these experiments did not identify any singular EWSR1 subdomain necessary for EWS-FLI1 activity, suggesting that there is significant functional redundancy between different parts of the EWSR1 prion-like domain.

Given these results, whether small EWSR1 fragments fused to the FLI1 C-terminal region were sufficient to recapitulate EWS-FLI1 function was tested (FIG. 12A). Strikingly, the fusion of the SYGQ1 fragment (37 amino acids) was sufficient to confer binding to wild-type EWSR1 and BRG1 (FIG. 13G), b-isox induced precipitation (FIG. 12B), and induction of expression of EWS-FLI1 target genes in MSCs (FIG. 12C). In line with these observations, the addition of a short EWSR1 fragment was sufficient to induce precipitation of purified GST-tagged SYGQ1-FLI1 in the in vitro sedimentation assays (FIG. 12D and FIG. 13H). Fusion of the SYGQ2 fragment (64 amino acids) to the FLI1 C-terminal region was also sufficient to recapitulate EWS-FLI1 function (FIGSS. 12A-C and FIG. 13G). In addition, a more detailed analysis of the SYGQ2 fusion showed that this mutant protein was able to bind GGAA repeat microsatellites and to recruit BAF complexes, leading to DNA accessibility and enhancer activation, as assessed by ATAC-seq and ChIP-seq for H3K27ac, respectively (FIG. 12E-G).

Finally, the distinct abilities of mutant EWS-FLI1 proteins to recapitulate EWSFLI1 mediated gene expression programs were evident by comparing RNA-seq expression profiles. MSCs expressing the short fragment fusion SYGQ2-FLI1 demonstrated clustering with EWS-FLI1 expressing cells (FIG. 12H) while MSCs expressing the tyrosine mutant EWS(YS37)-FLI1 clustered with control cells infected with an empty vector (FIG. 12H). In agreement with these results, cell growth arrest and phenotypic changes induced by EWS-FLI1 knockdown were rescued by the SYGQ2-FLI1 mutant protein but not by EWS(YS37)-FLI1 (FIG. 13I). These results thus demonstrate that even small isolated fragments of the EWSR1 prion-like domain are sufficient to recapitulate the function of the full EWS-FLI1 fusion on chromatin and to induce gene expression programs associated with GGAA microsatellites in Ewing sarcoma tumors.

### Example 8

Taken together, these studies elucidate critical mechanisms whereby EWSR1 contributes to the oncogenic activity of EWS-FLI1. They show that the EWSR1 prion-like domain mediates phase transition and leads to de novo activation of tumor specific enhancers through stable EWS-FLI1 binding to GGAA microsatellite repeats and BAF complex recruitment. These activities establish an oncogenic gene regulation program and represent a neomorphic property of the EWS-FLI1 fusion protein that is provided by EWSR1 and is not shared by the wild-type ETS factor FLI1 (FIGS. 14A and B). Remarkably, the fusion of even small, truncated fragments of the EWSR1 prion-like domain to FLI1 is sufficient to recapitulate the pioneer activity of EWS-FLI11, underscoring the critical and powerful role of this low-complexity domain in oncogenesis.

Low-complexity domains have been proposed to play a variety of roles in normal cellular functions and in disease states (Aguzzi and Altmeyer, 2016; March et al., 2016). In normal cells, proteins containing intrinsically disordered domains are believed to have the ability to form liquid-like compartments which, in the case of the FUS protein, have been observed in vivo in the cytoplasm upon stress and at sites of DNA damage in the nucleus (Patel et al., 2015). This process can be altered by mutations in the prion-like domain or local protein accumulation, as observed in Amyotrophic Lateral Sclerosis (ALS) where the pathological aggregation of low complexity proteins may be the result of the conversion from liquid to solid states (Couthouis et al., 2012; Couthouis et al., 2011; Patel et al., 2015; Sun et al., 2011). The results shown herein suggest that a similar phase transition mechanism could allow protein accumulation and stabilization at tumor specific DNA binding sites in Ewing sarcoma as well as in other tumor types involving fusions of FET family proteins with transcription factors.

The ability of EWS-FLI1 to bind and recruit BAF complexes to GGAA microsatellites represents a powerful mechanism for re-targeting these key chromatin remodeling complexes. It has previously been showed that EWS-FLI1 co-localizes with the acetyl-transferase P300 and the MLL complex at these sites (Riggi et al., 2014). While the interplay between these chromatin modifying complexes and the precise timing of events taking place at GGAA repeats remain to be determined, these results show that EWS-FLI1 binding and recruitment of the machinery necessary for de novo enhancer activation is dependent on the phase transition properties of the EWSR1 prion-like domain. Further characterization will be necessary to pinpoint the exact nature of these transitions at chromatin. Indeed, it will be of great interest to establish whether EWSFLI1 forms fibrils, amorphous aggregates or undergoes liquid-liquid phase separation in vivo, and to what extent the disruption of these neomorphic properties may be exploited from a therapeutic standpoint.

Mutations in the genes encoding BAF complex subunits are observed in many tumor types, suggesting that tumor-specific changes in BAF complex composition and function play important roles in human cancer (Kadoch et al., 2013). While the functional implications of these alterations are only beginning to be understood (Hodges et al., 2016; Kadoch et al., 2016), data shown herein suggest that one important consequence may be the redistribution of BAF complex activity toward genomic locations that promote oncogenesis (Kadoch and Crabtree, 2013). It is now shown that, in the absence of genetic alterations in BAF complex components, the EWS-FLI1 fusion protein utilizes an alternate mechanism to retarget of BAF complexes to distal regulatory elements. This in turn leads to the activation of target genes and the establishment of a tumor-specific regulatory network and transcriptional program. A similar neomorphic retargeting mechanism may also be operative in tumors driven by translocations involving other FET proteins and low complexity domains. Tumor specific re-targeting of BAF complexes by transcription factors and other proteins may thus play an important role in many tumor types in which BAF complexes are not genetically altered, thereby expanding the already wide-spanning role of BAF complexes in human cancer.

Alterations in BAF subunits may also be associated with changes in both subunit composition and configuration of the BAF complex. This has been demonstrated in synovial sarcoma, where the SS18-SSX translocation leads to the formation of a modified version of the BAF complex that incorporates the translocation protein (Kadoch and Crabtree, 2013). These results show that, despite strong interactions with BAF, EWSFLI1 is not incorporated into the core BAF complex and therefore does not affect BAF composition directly. Nevertheless, the recruitment of BAF complexes to GGAA repeats in association with the potential phase transitions induced by the EWSR1 prion-like motif may involve an unusual configuration that could be exploited for the development of new therapeutic approaches.

The experiments comparing EWS-FLI1, FLI1 and various mutant proteins in MSCs establish a direct link between the neomorphic properties conferred by the EWS prion-like domain and a tumor specific gene regulation program mediated by de novo enhancer activation at GGAA microsatellites. Indeed, FLI1 and the EWS(YS37)-FLI1 mutant protein exhibited substantial occupancy at canonical ETS binding sites, but in contrast to EWS-FLI1, were not able to bind GGAA repeats. This is in agreement with the observation that Ewing sarcoma cell lines were the only cell types with open, chromatin at EWS-FLI1-bound GGAA microsatellite repeats in a survey of 112 cell types analyzed by DNAse hypersensitivity (Riggi et al., 2014). These data included profiles for cells which express high levels of endogenous FLI1 and yet did not show signals at GGAA repeat sites. This is also consistent with recent studies suggesting that various types of stem cells may contain destabilized nucleosomes detected by FAIRE (formaldehyde-assisted isolation of regulatory elements) at repetitive elements but not open chromatin (Gomez et al., 2016). Importantly, prior electrophoretic mobility shift (EMSA) assays showed binding of both EWS-FLI1 and FLI1 to GGAA repeats in vitro (Gangwal et al., 2008), suggesting that the difference between these two proteins may only be evident in the appropriate in vivo chromatin context. The application of methods for direct high-throughput genome-wide analysis of chromatin thus provides opportunities for new insights into critical mechanisms of oncogenic gene regulation. In conclusion, this study demonstrates that a prion-like domain can confer neomorphic properties to fusion proteins that lead to re-targeting of key chromatin regulators and the establishment of an oncogenic gene regulatory program. Similar events mediated by FET family proteins or other intrinsically disordered proteins are likely to play important roles in generating tumor-specific regulatory elements in a variety of tumor types and may constitute attractive targets for therapeutic development.

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### REFERENCES

Aguzzi, A., and Altmeyer, M. (2016). Phase Separation: Linking Cellular Compartmentalization to Disease. Trends Cell Biol 26, 547-558.
Brohl, A. S., Solomon, D. A., Chang, W., Wang, J., Song, Y., Sindiri, S., Patidar, R., Hurd, L., Chen, L., Shern, J. F., et al. (2014). The genomic landscape of the Ewing Sarcoma family of tumors reveals recurrent STAG2 mutation. PLoS Genet 10, e1004475.
Couthouis, J., Hart, M. P., Erion, R., King, O. D., Diaz, Z., Nakaya, T., Ibrahim, F., Kim, H. J.,
Mojsilovic-Petrovic, J., Panossian, S., et al. (2012). Evaluating the role of the FUS/TLS-related gene EWSR1 in amyotrophic lateral sclerosis. Hum Mol Genet 21, 2899-2911.
Couthouis, J., Hart, M. P., Shorter, J., DeJesus-Hernandez, M., Erion, R., Oristano, R., Liu, A. X., Ramos, D., Jethava, N., Hosangadi, D., et al. (2011). A yeast functional screen predicts new candidate ALS disease genes. Proc Natl Acad Sci USA 108, 20881-20890.
Crompton, B. D., Stewart, C., Taylor-Weiner, A., Alexe, G., Kurek, K. C., Calicchio, M. L., Kiezun, A., Carter, S. L., Shukla, S. A., Mehta, S. S., et al. (2014). The genomic landscape of pediatric Ewing sarcoma. Cancer Discov 4, 1326-1341.
Delattre, O., Zucman, J., Plougastel, B., Desmaze, C., Melot, T., Peter, M., Kovar, H., Joubert, I., de Jong, P., Rouleau, G., et al. (1992). Gene fusion with an ETS DNA binding domain caused by chromosome translocation in human tumours. Nature 359, 162-165.
Embree, L. J., Azuma, M., and Hickstein, D. D. (2009). Ewing sarcoma fusion protein EWSR1/FLI1 interacts with EWSR1 leading to mitotic defects in zebrafish embryos and human cell lines. Cancer Res 69, 4363-4371.
Gangwal, K., Sankar, S., Hollenhorst, P. C., Kinsey, M., Haroldsen, S. C., Shah, A. A., Boucher, K. M., Watkins, W. S., Jorde, L. B., Graves, B. J., et al. (2008). Microsatellites as EWS/FLI response elements in Ewing's sarcoma. Proc Natl Acad Sci USA 105, 10149-10154.
Gomez, N. C., Hepperla, A. J., Dumitru, R., Simon, J. M., Fang, F., and Davis, I. J. (2016). Widespread Chromatin Accessibility at Repetitive Elements Links Stem Cells with Human Cancer. Cell Rep 17, 1607-1620.
Guillon, N., Tirode, F., Boeva, V., Zynovyev, A., Barillot, E., and Delattre, O. (2009). The oncogenic EWS-FLI1 protein binds in vivo GGAA microsatellite sequences with potential transcriptional activation function. PLoS One 4, e4932.
Han, T. W., Kato, M., Xie, S., Wu, L. C., Mirzaei, H., Pei, J., Chen, M., Xie, Y., Allen, J., Xiao, G., et al. (2012). Cell-free formation of RNA granules: bound RNAs identify features and components of cellular assemblies. Cell 149, 768-779.
Herrero-Martin, D., Fourtouna, A., Niedan, S., Riedmann, L. T., Schwentner, R., and Aryee, D. N. (2011). Factors Affecting EWS-FLI1 Activity in Ewing's Sarcoma. Sarcoma 2011, 352580.
Hodges, C., Kirkland, J. G., and Crabtree, G. R. (2016). The Many Roles of BAF (mSWI/SNF) and PBAF Complexes in Cancer. Cold Spring Harb Perspect Med 6.
Kadoch, C., and Crabtree, G. R. (2013). Reversible disruption of mSWI/SNF (BAF) complexes by the SS18-SSX oncogenic fusion in synovial sarcoma. Cell 153, 71-85.
Kadoch, C., and Crabtree, G. R. (2015). Mammalian SWI/SNF chromatin remodeling complexes and cancer: Mechanistic insights gained from human genomics. Sci Adv 1, e1500447.
Kadoch, C., Hargreaves, D. C., Hodges, C., Elias, L., Ho, L., Ranish, J., and Crabtree, G. R. (2013). Proteomic and bioinformatic analysis of mammalian SWI/SNF complexes identifies extensive roles in human malignancy. Nat Genet 45, 592-601.
Kadoch, C., Williams, R. T., Calarco, J. P., Miller, E. L., Weber, C. M., Braun, S. M., Pulice, J. L., Chory, E. J., and Crabtree, G. R. (2016). Dynamics of BAF-Polycomb complex opposition on heterochromatin in normal and oncogenic states. Nat Genet.
Kato, M., Han, T. W., Xie, S., Shi, K., Du, X., Wu, L. C., Mirzaei, H., Goldsmith, E. J., Longgood, J., Pei, J., et al. (2012). Cell-free formation of RNA granules: low complexity sequence domains form dynamic fibers within hydrogels. Cell 149, 753-767.
Kovar, H. (2011). Dr. Jekyll and Mr. Hyde: The Two Faces of the FUS/EWS/TAF15 Protein Family. Sarcoma 2011, 837474.
Kwon, I., Kato, M., Xiang, S., Wu, L., Theodoropoulos, P., Mirzaei, H., Han, T., Xie, S., Corden, J. L., and McKnight, S. L. (2013). Phosphorylation-regulated binding of RNA polymerase II to fibrous polymers of low-complexity domains. Cell 155, 1049-1060.
Lander, E. S. (2011). Initial impact of the sequencing of the human genome. Nature 470, 187-197.
Lawrence, M. S., Stojanov, P., Polak, P., Kryukov, G. V., Cibulskis, K., Sivachenko, A., Carter, S. L., Stewart, C., Mermel, C. H., Roberts, S. A., et al. (2013). Mutational heterogeneity in cancer and the search for new cancer-associated genes. Nature 499, 214-218.
March, Z. M., King, O. D., and Shorter, J. (2016). Prion-like domains as epigenetic regulators, scaffolds for subcellular organization, and drivers of neurodegenerative disease. Brain Res 1647, 9-18.
Mertens, F., Antonescu, C. R., and Mitelman, F. (2016). Gene fusions in soft tissue tumors: Recurrent and overlapping pathogenetic themes. Genes Chromosomes Cancer 55, 291-310.
Patel, A., Lee, H. O., Jawerth, L., Maharana, S., Jahnel, M., Hein, M. Y., Stoynov, S., Mahamid, J., Saha, S., Franzmann, T. M., et al. (2015). A Liquid-to-Solid Phase Transition of the ALS Protein FUS Accelerated by Disease Mutation. Cell 162, 1066-1077.
Patel, M., Simon, J. M., Iglesia, M. D., Wu, S. B., McFadden, A. W., Lieb, J. D., and Davis, I. J. (2012). Tumor-specific retargeting of an oncogenic transcription factor chimera results in dysregulation of chromatin and transcription. Genome Res 22, 259-270.
Phelan, M. L., Sif, S., Narlikar, G. J., and Kingston, R. E. (1999). Reconstitution of a core chromatin remodeling complex from SWI/SNF subunits. Mol Cell 3, 247-253.
Riggi, N., Cironi, L., Provero, P., Suva, M. L., Kaloulis, K., Garcia-Echeverria, C., Hoffmann, F., Trumpp, A., and Stamenkovic, I. (2005). Development of Ewing's sarcoma from primary bone marrow-derived mesenchymal progenitor cells. Cancer Res 65, 11459-11468.
Riggi, N., Knoechel, B., Gillespie, S. M., Rheinbay, E., Boulay, G., Suva, M. L., Rossetti, N. E., Boonseng, W. E., Oksuz, O., Cook, E. B., et al. (2014). EWS-FLI1 utilizes divergent chromatin remodeling mechanisms to directly activate or repress enhancer elements in Ewing sarcoma. Cancer Cell 26, 668-681.
Riggi, N., Suva, M. L., Suva, D., Cironi, L., Provero, P., Tercier, S., Joseph, J. M., Stehle, J. C., Baumer, K., Kindler, V., et al. (2008). EWS-FLI-1 expression triggers a Ewing's sarcoma initiation program in primary human mesenchymal stem cells. Cancer Res 68, 2176-2185.
Roberts, C. W., Leroux, M. M., Fleming, M. D., and Orkin, S. H. (2002). Highly penetrant, rapid tumorigenesis through conditional inversion of the tumor suppressor gene Snf5. Cancer Cell 2, 415-425.
Schwartz, J. C., Wang, X., Podell, E. R., and Cech, T. R. (2013). RNA seeds higher-order assembly of FUS protein. Cell Rep 5, 918-925.
Spahn, L., Siligan, C., Bachmaier, R., Schmid, J. A., Aryee, D. N., and Kovar, H. (2003). Homotypic and heterotypic interactions of EWS, FLI1 and their oncogenic fusion protein. Oncogene 22, 6819-6829.
Sun, Z., Diaz, Z., Fang, X., Hart, M. P., Chesi, A., Shorter, J., and Gitler, A. D. (2011). Molecular determinants and genetic modifiers of aggregation and toxicity for the ALS disease protein FUS/TLS. PLoS Biol 9, e1000614.
Tanaka, K., Iwakuma, T., Harimaya, K., Sato, H., and Iwamoto, Y. (1997). EWS-FLI1 antisense oligodeoxynucleotide inhibits proliferation of human Ewing's sarcoma and primitive neuroectodermal tumor cells. J Clin Invest 99, 239-247.
Thomsen, C., Grundevik, P., Elias, P., Stahlberg, A., and Aman, P. (2013). A conserved N-terminal motif is required for complex formation between FUS, EWSR1, TAF15 and their oncogenic fusion proteins. FASEB J 27, 4965-4974.
Tirode, F., Surdez, D., Ma, X., Parker, M., Le Deley, M. C., Bahrami, A., Zhang, Z., Lapouble, E., Grossetete-Lalami, S., Rusch, M., et al. (2014). Genomic landscape of Ewing sarcoma defines an aggressive subtype with co-association of STAG2 and TP53 mutations. Cancer Discov 4, 1342-1353.
Tomazou, E. M., Sheffield, N.C., Schmidl, C., Schuster, M., Schonegger, A., Datlinger, P., Kubicek, S., Bock, C., and Kovar, H. (2015). Epigenome mapping reveals distinct modes of gene regulation and widespread enhancer reprogramming by the oncogenic fusion protein EWS-FLI1. Cell Rep 10, 1082-1095.
Versteege, I., Sevenet, N., Lange, J., Rousseau-Merck, M. F., Ambros, P., Handgretinger, R., Aurias, A., and Delattre, O. (1998). Truncating mutations of hSNF5/INI1 in aggressive paediatric cancer. Nature 394, 203-206.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject afflicted with cancer comprising administering to the subject a therapeutically effective amount of an agent that inhibits binding of a FET-ETS fusion protein to a BAF complex.

2. The method of claim 1, wherein the agent is a small molecule inhibitor, a small molecule degrader, CRISPR guide RNA (gRNA), RNA interfering agent, oligonucleotide, peptide or peptidomimetic inhibitor, aptamer, antibody, or intrabody.

3. The method of claim 2, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

4. The method of claim 2, wherein the agent comprises an antibody and/or intrabody, or an antigen binding fragment thereof, which specifically binds to the FET-ETS fusion protein or the BAF complex.

5. The method of claim 4, wherein the antibody and/or intrabody, or antigen binding fragment thereof, is chimeric, humanized, composite, or human.

6. The method of claim 4 or 5, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

7. The method of any one of claims 1-6, further comprising administering to the subject an immunotherapy and/or cancer therapy, optionally wherein the immunotherapy and/or cancer therapy is administered before, after, or concurrently with the agent.

8. The method of claim 7, wherein the immunotherapy is cell-based.

9. The method of claim 7, wherein the immunotherapy comprises a cancer vaccine and/or virus.

10. The method of claim 7, wherein the immunotherapy inhibits an immune checkpoint.

11. The method of claim 10, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

12. The method of claim 7, wherein the cancer therapy is selected from the group consisting of radiation, a radiosensitizer, and a chemotherapy.

13. The method of any one of claims 1-12, wherein the agent reduces the number of viable or proliferating cells in the cancer, and/or reduces the volume or size of a tumor comprising the cancer cells.

14. The method of any one of claims 1-13, further comprising administering to the subject at least one additional therapeutic agent or regimen for treating the cancer.

15. A method of reducing viability or proliferation of cancer cells comprising contacting the cancer cells with an agent that inhibits binding of a FET-ETS fusion protein to a BAF complex.

16. The method of claim 15, wherein the agent is a small molecule inhibitor, a small molecule degrader, CRISPR guide RNA (gRNA), RNA interfering agent, oligonucleotide, peptide or peptidomimetic inhibitor, aptamer, antibody, or intrabody.

17. The method of claim 16, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

18. The method of claim 17, wherein the agent comprises an antibody and/or intrabody, or an antigen binding fragment thereof, which specifically binds to the FET-ETS fusion protein or the BAF complex.

19. The method of claim 18, wherein the antibody and/or intrabody, or antigen binding fragment thereof, is chimeric, humanized, composite, or human.

20. The method of claim 18 or 19, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

21. The method of any one of claims 15-20, further comprising contacting the cancer cells with an immunotherapy and/or cancer therapy, optionally wherein the immunotherapy and/or cancer therapy is administered before, after, or concurrently with the agent.

22. The method of claim 21, wherein the immunotherapy is cell-based.

23. The method of claim 21, wherein the immunotherapy comprises a cancer vaccine and/or virus.

24. The method of claim 21, wherein the immunotherapy inhibits an immune checkpoint.

25. The method of claim 24, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

26. The method of claim 21, wherein the cancer therapy is selected from the group consisting of radiation, a radiosensitizer, and a chemotherapy.

27. The method of any one of claims 1-26, wherein the agent decreases binding of the BAF complex to at least one FET-ETS fusion protein-bound GGAA repeat enhancer.

28. The method of claim 27, wherein the FET-ETS fusion protein-bound GGAA repeat enhancer is associated with a gene selected from the group consisting of KIT, CCND1, NKX2-2, SOX2, NR0B1, EZH2, and LINC00221.

29. The method of any one of claims 1-28, wherein the agent decreases expression of at least one target gene of the FET-ETS fusion protein.

30. The method of claim 29, wherein the target gene of the FET-ETS fusion protein is selected from the group consisting of NKX2-2, NPY1R, PPP1RIA, KIT, LOXHD 1, MAFB, and NGFR.

31. A method of treating a subject afflicted with cancer comprising administering to the subject a therapeutically effective amount of an agent that inhibits binding of a BAF complex to at least one FET-ETS fusion protein-bound GGAA repeat enhancer.

32. The method of claim 31, wherein the agent is a small molecule inhibitor, a small molecule degrader, CRISPR guide RNA (gRNA), RNA interfering agent, oligonucleotide, peptide or peptidomimetic inhibitor, aptamer, antibody, or intrabody.

33. The method of claim 32, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

34. The method of claim 32, wherein the agent comprises an antibody and/or intrabody, or an antigen binding fragment thereof, which specifically binds to the FET-ETS fusion protein or the BAF complex.

35. The method of claim 34, wherein the antibody and/or intrabody, or antigen binding fragment thereof, is chimeric, humanized, composite, or human.

36. The method of claim 34 or 35, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

37. The method of any one of claims 31-36, further comprising administering to the subject an immunotherapy and/or cancer therapy, optionally wherein the immunotherapy and/or cancer therapy is administered before, after, or concurrently with the agent.

38. The method of claim 37, wherein the immunotherapy is cell-based.

39. The method of claim 37, wherein the immunotherapy comprises a cancer vaccine and/or virus.

40. The method of claim 37, wherein the immunotherapy inhibits an immune checkpoint.

41. The method of claim 40, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

42. The method of claim 37, wherein the cancer therapy is selected from the group consisting of radiation, a radiosensitizer, and a chemotherapy.

43. The method of any one of claims 31-42, wherein the agent reduces the number of viable or proliferating cells in the cancer, and/or reduces the volume or size of a tumor comprising the cancer cells.

44. The method of any one of claims 31-43, further comprising administering to the subject at least one additional therapeutic agent or regimen for treating the cancer.

45. A method of reducing viability or proliferation of cancer cells comprising contacting the cancer cells with an agent that inhibits binding of a BAF complex to at least one FET-ETS fusion protein-bound GGAA repeat enhancer.

46. The method of claim 45, wherein the agent is a small molecule inhibitor, a small molecule degrader, CRISPR guide RNA (gRNA), RNA interfering agent, oligonucleotide, peptide or peptidomimetic inhibitor, aptamer, antibody, or intrabody.

47. The method of claim 46, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

48. The method of claim 46, wherein the agent comprises an antibody and/or intrabody, or an antigen binding fragment thereof, which specifically binds to the FET-ETS fusion protein or the BAF complex.

49. The method of claim 48, wherein the antibody and/or intrabody, or antigen binding fragment thereof, is chimeric, humanized, composite, or human.

50. The method of claim 48 or 49, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

51. The method of any one of claims 45-50, further comprising contacting the cancer cells with an immunotherapy and/or cancer therapy, optionally wherein the immunotherapy and/or cancer therapy is administered before, after, or concurrently with the agent.

52. The method of claim 51, wherein the immunotherapy is cell-based.

53. The method of claim 51, wherein the immunotherapy comprises a cancer vaccine and/or virus.

54. The method of claim 51, wherein the immunotherapy inhibits an immune checkpoint.

55. The method of claim 54, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

56. The method of claim 51, wherein the cancer therapy is selected from the group consisting of radiation, a radiosensitizer, and a chemotherapy.

57. The method of any one of claims 31-56, wherein the FET-ETS fusion protein-bound GGAA repeat enhancer is associated with a gene selected from the group consisting of KIT, CCND1, NKX2-2, SOX2, NR0B1, EZH2, and LINC00221.

58. The method of any one of claims 31-57, wherein the agent inhibits the binding of the FET-ETS fusion protein to the BAF complex.

59. The method of any one of claims 31-58, wherein the agent decreases expression of at least one target gene of the FET-ETS fusion protein.

60. The method of claim 59, wherein the target gene of the FET-ETS fusion protein is selected from the group consisting of NKX2-2, NPY1R, PPP1R1A, KIT, LOXHD1, MAFB, and NGFR.

61. The method of any one of claims 1-60, wherein the agent is administered in a pharmaceutically acceptable formulation.

62. A method of assessing the efficacy of the agent of claim 1 or claim 31 for treating cancer in a subject, comprising:
a) detecting in a subject sample at a first point in time the amount of at least one gene selected from the group consisting of NKX2-2, NPY1R, PPP1R1A, KIT, LOXHD1, MAFB, and NGFR;
b) repeating step a) during at least one subsequent point in time after administration of the agent; and
c) comparing the amount detected in steps a) and b), wherein the absence of, or a significant decrease in amount of at least one gene selected from the group consisting of NKX2-2, NPY1R, PPP1R1A, KIT, LOXHD1, MAFB, and NGFR in the subsequent sample as compared to the amount in the sample at the first point in time, indicates that the agent treats cancer in the subject.

63. The method of claim 62, wherein between the first point in time and the subsequent point in time, the subject has undergone treatment, completed treatment, and/or is in remission for the cancer.

64. The method of claim 62 or 63, wherein the first and/or at least one subsequent sample is selected from the group consisting of ex vivo and in vivo samples.

65. The method of any one of claims 62-64, wherein the first and/or at least one subsequent sample is obtained from an animal model of the cancer.

66. The method of any one of claims 62-65, wherein the first and/or at least one subsequent sample is a portion of a single sample or pooled samples obtained from the subject.

67. The method of any one of claims 62-66, wherein the sample comprises cells, serum, peritumoral tissue, and/or intratumoral tissue obtained from the subject.

68. The method of any one of claims 62-67, further comprising determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

69. The method of any one of claims 1-68, wherein the agent is a BAF complex inhibitor.

70. The method of claim 69, wherein the BAF complex inhibitor is selected form the group consisting of Bromosporine, LP99, I-BRD9, BI-9564, BI-7273, GSK-39, dBRD9, and PFI-3.

71. A cell-based assay for screening for agents that reduce viability or proliferation of a cancer cell comprising contacting the cancer cell with a test agent, and determining the ability of the test agent to decrease (1) binding of a FET-ETS fusion protein to a BAF complex; (2) binding of a BAF complex to at least one FET-ETS fusion protein-bound GGAA repeat enhancer; and/or (3) expression of at least one target gene of the FET-ETS fusion protein.

72. The cell-based assay of claim 71, wherein the step of contacting occurs in vivo, ex vivo, or in vitro.

73. The cell-based assay of claim 71 or 72, wherein the FET-ETS fusion protein-bound GGAA repeat enhancer is associated with a gene selected from the group consisting of KIT, CCND1, NKX2-2, SOX2, NR0B1, EZH2, and LINC00221.

74. The cell-based assay of any one of claims 71-73, wherein the target gene of the FET-ETS fusion protein is selected from the group consisting of NKX2-2, NPY1R, PPP1R1A, KIT, LOXHD1, MAFB, and NGFR.

75. The cell-based assay of any one of claims 71-74, further comprising determining a reduction in the viability or proliferation of the cancer cells.

76. An in vitro assay for screening for agents that reduce viability or proliferation of cancer cells comprising:
a) mixing a FET-ETS fusion protein-bound GGAA repeat enhancer, a FET-ETS fusion protein, and a BAF complex together;
b) adding a test agent to the mixture; and
c) determining the ability of the test agent to decrease binding of the FET-ETS fusion protein to the BAF complex, and/or binding of the BAF complex to the FET-ETS fusion protein-bound GGAA repeat enhancer.

77. The in vitro assay of claim 76, wherein the FET-ETS fusion protein-bound GGAA repeat enhancer is associated with a gene selected from the group consisting of KIT, CCND1, NKX2-2, SOX2, NR0B1, EZH2, and LINC00221.

78. The method or assay of any one of claims 1-77, wherein the BAF complex is a human BAF complex.

79. The method or assay of any one of claims 1-78, wherein the FET-ETS fusion protein binds to at least one subunit of the BAF complex, wherein the subunit is selected from the group consisting of SMARCA2, SMARCA4/BRG1, SMARCB1/BAF47, SMARCC1/BAF155, SMARCC2/BAF170, SMARCD1/BAF60A, SMARCD2, SMARCE1/BAF 157, DPF2/BAF45D, ARID1A/BAF250A, ARID1B/BAF250B, SS18, and ACTL6A/BAF53A.

80. The method or assay of any one of claims 1-79, wherein the FET-ETS fusion protein consists of an N-terminal portion of a FET protein and a C-terminal portion of a ETS protein.

81. The method or assay of any one of claims 1-80, wherein the binding of the FET-ETS fusion protein to the BAF complex is dependent on a prion-like domain of the N-terminal portion of the FET protein.

82. The method or assay of any one of claims 1-81, wherein the binding of the FET-ETS fusion protein to the BAF complex is dependent on the tyrosine residues in the prion-like domain.

83. The method or assay of claim 80, wherein the FET protein is selected from the group consisting of FUS, TAF15, and EWSR1.

84. The method or assay of claim 80, wherein the ETS protein is selected from the group consisting of FLI1, ERG, ETV1, and ETS1.

85. The method or assay of any one of claims 1-84, wherein the subject is an animal model of the cancer.

86. The method or assay of any one of claims 1-85, wherein the subject is a mammal.

87. The method or assay of claim 86, wherein the mammal is a human.

88. The method or assay of any one of claims 1-87, wherein the cancer is selected from the group consisting of leukemia, Ewing sacoma and primitive neuroectodermal tumor (PNET).

89. The method or assay of any one of claims 1-88, wherein the FET-ETS fusion protein is EWS-FLI1, and the cancer is Ewing sacoma or primitive neuroectodermal tumor (PNET).


SEQUENCE LISTING (76 sequences)

SEQ ID NO: 1 - protein, 498 aa, Homo sapiens
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 2 - protein, 520 aa, Homo sapiens
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQSSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 3 - protein, 264 aa, Homo sapiens
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQ

SEQ ID NO: 4 - protein, 233 aa, Homo sapiens
PSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 5 - protein, 498 aa, Homo sapiens
MASTDYSTYSQAAAQQGSSAYTAQPTQGYAQTTQAYGQQSSGTYGQPTDVSYTQAQTTATYGQTAYATSSGQPPTGYTTPTAPQAYSQPVQGSGTGAYDTTTATVTTTQASYAAQSAYGTQPAYPAYGQQPAATAPTRPQDGNKPTETSQPQSSTGGYNQPSLGSGQSNYSYPQVPGSYPMQPVTAPPSYPPTSSSSTQPTSYDQSSSSQQNTYGQPSSSGQQSSSGQQSSSGQQPPTSYPPQTGSSSQAPSQYSQQSSSSGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 6 - protein, 498 aa, Homo sapiens
MASTDSSTSSQAAAQQGSSASTAQPTQGSAQTTQASGQQSSGTSGQPTDVSSTQAQTTATSGQTASATSSGQPPTGSTTPTAPQASSQPVQGSGTGASDTTTATVTTTQASSAAQSASGTQPASPASGQQPAATAPTRPQDGNKPTETSQPQSSTGGSNQPSLGSGQSNSSSPQVPGSSPMQPVTAPPSSPPTSSSSTQPTSSDQSSSSQQNTSGQPSSSGQQSSSGQQSSSGQQPPTSSPPQTGSSSQAPSQSSQQSSSSGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 7 - protein, 271 aa, Homo sapiens
YGQQSYGTYGQPTDVSYTQAQTTATYGQTAYATSYGQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 8 - protein, 298 aa, Homo sapiens
TSYDQSSYSQQNTYGQPSSYGQQSSYGQQSSYGQQPPTSYPPQTGSYSQAPSQYSQQSSSYGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 9 - protein, 623 aa, Homo sapiens
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAWGKPIPNPLLGLDSTPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 10 - DNA, 1497 bp, Homo sapiens
atggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactaa

SEQ ID NO: 11 - DNA, 1563 bp, Homo sapiens
atggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcactgctggcctataatacaacctcccacaccgaccaatcctcacgattgagtgtcaaagaagacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactag

SEQ ID NO: 12 - DNA, 702 bp, Homo sapiens
ccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactag

SEQ ID NO: 13 - DNA, 1497 bp, Homo sapiens
atggcgtccacggattacagtacctatagccaagctgcagcgcagcagggctccagtgcttacaccgcccagcccactcaaggatatgctcaaactactcaagcctatggtcagcagtcttccggcacctatggccagcctaccgacgtgagctatacacaggctcaaacaacggcgacttatgggcaaacagcctatgcgacaagttcaggacaaccgcctaccggatatactacgccgaccgccccacaggcctattcccagcctgtccaaggttctgggactggcgcttatgacactactaccgccacggtaactaccacccaagcgtcctatgccgcccagagcgcgtatggcacccaacctgcgtatccggcatatggacaacaacccgcagccaccgcccccacgcgccctcaggatggcaataaacccacggaaacatcccaaccccaatcatccacgggcggatacaaccagccatcactcggctcagggcaaagtaattacagctatcctcaagtgccgggttcctacccgatgcaacccgtcactgcaccgccgtcttacccgcccactagctcttcctctacacagccgactagttatgatcagagcagttcctctcagcagaacacctatgggcaaccgagcagctctggacagcagagtagctctggtcaacaaagcagctctgggcagcagcctcccactagttacccaccccaaactggatcctccagccaagctccaagtcaatatagccaacagagcagcagctccgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactaa

SEQ ID NO: 14 - DNA, 1497 bp, Homo sapiens
atggcgtccacggattccagtacctctagccaagctgcagcgcagcagggctccagtgcttccaccgcccagcccactcaaggatccgctcaaactactcaagcctccggtcagcagtcttcaggcacctccggccagcctaccgacgtgagcagtacacaggctcaaacaacggcgactagcgggcaaacagcctccgcgacaagttcaggacaaccgcctaccggatctactacgccgaccgccccacaggccagttcccagcctgtccaaggttctgggactggcgctagtgacactactaccgccacggtaactaccacccaagcgtccagtgccgcccagagcgcgtcaggcacccaacctgcgtctccggcaagtggacaacaacccgcagccaccgcccccacgcgccctcaggatggcaataaacccacggaaacatcccaaccccaatcatccacgggcggatcaaaccagccatcactcggctcagggcaaagtaattccagctcccctcaagtgccgggttcctccccgatgcaacccgtcactgcaccgccgtctagcccgcccactagctcttcctctacacagccgactagttctgatcagagcagttcctctcagcagaacacctctgggcaaccgagcagctctggacagcagagtagctctggtcaacaaagcagctctgggcagcagcctcccactagttccccaccccaaactggatcctccagccaagctccaagtcaatctagccaacagagcagcagctccgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactaa

SEQ ID NO: 15 - DNA, 816 bp, Homo sapiens
tatgggcaacaaagctatggaacctatggacagcccactgatgtcagctatacccaggctcagaccactgcaacctatgggcagaccgcctatgcaacttcttatggacagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactaa

SEQ ID NO: 16 - DNA, 897 bp, Homo sapiens
actagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactaa

SEQ ID NO: 17 - DNA, 1872 bp, Homo sapiens
atgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggggtaagcctatccctaaccctctcctcggtctcgattctacgccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactag

SEQ ID NO: 18 - DNA, 22 bp, Artificial Sequence
  /note=“Description of Artificial Sequence Synthetic primer sequence”
caccgaaggaggcacttacaca

SEQ ID NO: 19 - DNA, 22 bp, Artificial Sequence
  /note=“Description of Artificial Sequence Synthetic primer sequence”
tgccattcacgagcctgtcgta

SEQ ID NO: 20 - DNA, 20 bp, Artificial Sequence
  /note=“Description of Artificial Sequence Synthetic primer sequence”
tggcgatgagcaccactact

SEQ ID NO: 21 - DNA, 20 bp, Artificial Sequence
  /note=“Description of Artificial Sequence Synthetic primer sequence”
ggaggacttggctggagatt

SEQ ID NO: 22 - DNA, 22 bp, Artificial Sequence
  /note=“Description of Artificial Sequence Synthetic primer sequence”
atatccgagccgaagcagacac

SEQ ID NO: 23 - DNA, 22 bp, Artificial Sequence
  /note=“Description of Artificial Sequence Synthetic primer sequence”
cggcatttctcctggtattccg

SEQ ID NO: 24 - DNA, 24 bp, Artificial Sequence
  /note=“Description of Artificial Sequence Synthetic primer sequence”
tgttggaggcttgatggaaaaacc

SEQ ID NO: 25 - DNA, 22 bp, Artificial Sequence
  /note=“Description of Artificial Sequence Synthetic primer sequence”
cggattctgacaggtgttcacc

SEQ ID NO: 26 - DNA, 22 bp, Artificial Sequence
  /note=“Description of Artificial Sequence Synthetic primer sequence”
tgagggtatcgcagagaacgga

SEQ ID NO: 27 - DNA, 22 bp, Artificial Sequence
  /note=“Description of Artificial Sequence Synthetic primer sequence”
cggtcacttatcctgtggctgg

SEQ ID NO: 28 - DNA, 23 bp, Artificial Sequence
  /note=“Description of Artificial Sequence Synthetic primer sequence”
ccatcggactctcataggttgtc

SEQ ID NO: 29 - DNA, 24 bp, Artificial Sequence
  /note=“Description of Artificial Sequence Synthetic primer sequence”
gacctgtacttattgtctctcatc

SEQ ID NO: 30 - DNA, 19 bp, Artificial Sequence
  /note=“Description of Artificial Sequence Synthetic primer sequence”
gacgcagctcattcagcag

SEQ ID NO: 31 - DNA, 21 bp, Artificial Sequence
  /note=“Description of Artificial Sequence Synthetic primer sequence”
ctcgcacttgaccttgtaggc

SEQ ID NO: 32 - DNA, 22 bp, Artificial Sequence
  /note=“Description of Artificial Sequence Synthetic primer sequence”
cacagaagtggagtcaaggctg

SEQ ID NO: 33 - DNA, 22 bp, Artificial Sequence
  /note=“Description of Artificial Sequence Synthetic primer sequence”
ttggctcccttggaatccagtg

SEQ ID NO: 34 - DNA, 20 bp, Artificial Sequence
  /note=“Description of Artificial Sequence Synthetic primer sequence”
aggggaccgtgctctttaac

SEQ ID NO: 35 - DNA, 20 bp, Artificial Sequence
  /note=“Description of Artificial Sequence Synthetic primer sequence”
ctgagttccccactggagtc

SEQ ID NO: 36 - DNA, 22 bp, Artificial Sequence
  /note=“Description of Artificial Sequence Synthetic primer sequence”
cctcatccctgtctattgctcc

SEQ ID NO: 37 - DNA, 22 bp, Artificial Sequence
  /note=“Description of Artificial Sequence Synthetic primer sequence”
gttggctccttgcttgttctgc

SEQ ID NO: 38 - DNA, 22 bp, Artificial Sequence
  /note=“Description of Artificial Sequence Synthetic primer sequence”
ttcagccgaggctctcctaatg

SEQ ID NO: 39 - DNA, 22 bp, Artificial Sequence
  /note=“Description of Artificial Sequence Synthetic primer sequence”
gcctgaaaccttttccgcacac

SEQ ID NO: 40 - DNA, 18 bp, Artificial Sequence
  /note=“Description of Artificial Sequence Synthetic primer sequence”
cagcgacaacccgtacac

SEQ ID NO: 41 - DNA, 21 bp, Artificial Sequence
  /note=“Description of Artificial Sequence Synthetic primer sequence”
gacttggagcttgagtcctga

SEQ ID NO: 42 - DNA, 22 bp, Artificial Sequence
  /note=“Description of Artificial Sequence Synthetic primer sequence”
gagggacacatcaagattgccg

SEQ ID NO: 43 - DNA, 22 bp, Artificial Sequence
  /note=“Description of Artificial Sequence Synthetic primer sequence”
caccaatccacggacttcccat

SEQ ID NO: 44 - DNA, 20 bp, Artificial Sequence
  /note=“Description of Artificial Sequence Synthetic primer sequence”
tgggaaagtacacggggata

SEQ ID NO: 45 - DNA, 20 bp, Artificial Sequence
  /note=“Description of Artificial Sequence Synthetic primer sequence”
tattgaccaagggcattcac

SEQ ID NO: 46 - DNA, 20 bp, Artificial Sequence
  /note=“Description of Artificial Sequence Synthetic primer sequence”
ggtctcctctgacttcaaca

SEQ ID NO: 47 - DNA, 20 bp, Artificial Sequence
  /note=“Description of Artificial Sequence Synthetic primer sequence”
gtgagggtctctctcttcct

SEQ ID NO: 48 - DNA, 24 bp, Artificial Sequence
  /note=“Description of Artificial Sequence Synthetic primer sequence”
cattatgctgaggatttggaaagg

SEQ ID NO: 49 - DNA, 22 bp, Artificial Sequence
  /note=“Description of Artificial Sequence Synthetic primer sequence”
cttgagcacacagagggctaca

SEQ ID NO: 50 - DNA, 22 bp, Artificial Sequence
  /note=“Description of Artificial Sequence Synthetic primer sequence”
accatctacggcgaggagtatg

SEQ ID NO: 51 - DNA, 22 bp, Artificial Sequence
  /note=“Description of Artificial Sequence Synthetic primer sequence”
cccaggtcaatggcatagatgg

SEQ ID NO: 52 - DNA, 23 bp, Artificial Sequence
  /note=“Description of Artificial Sequence Synthetic primer sequence”
gagaatggactgaacaggagacc

SEQ ID NO: 53 - DNA, 22 bp, Artificial Sequence
  /note=“Description of Artificial Sequence Synthetic primer sequence”
gggtcctcaatgggaagtctca

SEQ ID NO: 54 - DNA, 23 bp, Artificial Sequence
  /note=“Description of Artificial Sequence Synthetic primer sequence”
acacaccatttagtaaaggccaa

SEQ ID NO: 55 - DNA, 25 bp, Artificial Sequence
  /note=“Description of Artificial Sequence Synthetic primer sequence”
aggaatttggggatttctcaatcaa

SEQ ID NO: 56 - DNA, 20 bp, Artificial Sequence
  /note=“Description of Artificial Sequence Synthetic primer sequence”
gaagtgcaccctatgccagt

SEQ ID NO: 57 - DNA, 20 bp, Artificial Sequence
  /note=“Description of Artificial Sequence Synthetic primer sequence”
tcctctgtgggggttatcca

SEQ ID NO: 58 - DNA, 25 bp, Artificial Sequence
  /note=“Description of Artificial Sequence Synthetic primer sequence”
gattctgtatcagctggtatatacc

SEQ ID NO: 59 - DNA, 20 bp, Artificial Sequence
  /note=“Description of Artificial Sequence Synthetic primer sequence”
gcatcaggaagcctggatcc

SEQ ID NO: 60 - DNA, 20 bp, Artificial Sequence
  /note=“Description of Artificial Sequence Synthetic primer sequence”
ttctttttggtctgctggaa

SEQ ID NO: 61 - DNA, 20 bp, Artificial Sequence
  /note=“Description of Artificial Sequence Synthetic primer sequence”
cgtgcctgacactcttcaac

SEQ ID NO: 62 - DNA, 20 bp, Artificial Sequence
  /note=“Description of Artificial Sequence Synthetic primer sequence”
agcccttccaggaacagaac

SEQ ID NO: 63 - DNA, 20 bp, Artificial Sequence
  /note=“Description of Artificial Sequence Synthetic primer sequence”
ttcccaaatacccttcagca

SEQ ID NO: 64 - DNA, 20 bp, Artificial Sequence
  /note=“Description of Artificial Sequence Synthetic primer sequence”
atattggagccccaagggtt

SEQ ID NO: 65 - DNA, 20 bp, Artificial Sequence
  /note=“Description of Artificial Sequence Synthetic primer sequence”
ctcattagcagcggtggcac

SEQ ID NO: 66 - DNA, 21 bp, Artificial Sequence
  /note=“Description of Artificial Sequence Synthetic shRNA sequence”
cgtcatgttctggtttgagat

SEQ ID NO: 67 - DNA, 21 bp, Artificial Sequence
  /note=“Description of Artificial Sequence Synthetic shRNA sequence”
cccaccacatttacccatatt

SEQ ID NO: 68 - DNA, 21 bp, Artificial Sequence
  /note=“Description of Artificial Sequence Synthetic shRNA sequence”
gcaggatattagctccttata

SEQ ID NO: 69 - DNA, 21 bp, Artificial Sequence
  /note=“Description of Artificial Sequence Synthetic shRNA sequence”
ggcataggccttagcagtaac

SEQ ID NO: 70 - protein, 4 aa, Artificial Sequence
  /note=“Description of Artificial Sequence Synthetic EWSR1 prion-like domain sequence”
SYGQ

SEQ ID NO: 71 - DNA, 14 bp, Artificial Sequence
  /note=“Description of Artificial Sequence Synthetic AP2 motif sequence”
  misc_feature (1)..(1): n is a, c, g, or t
  misc_feature (2)..(2): s is c or g
  misc_feature (4)..(4): y is c or t
  misc_feature (5)..(5): h is a, c, or t
  misc_feature (6)..(6): n is a, c, g, or t
  misc_feature (7)..(7): d is a, g, or t
  misc_feature (10)..(10): s is c or g
  misc_feature (11)..(11): h is a, c, or t
  misc_feature (12)..(12): n is a, c, g, or t
  misc_feature (13)..(13): n is a, c, g, or t
  misc_feature (14)..(14): n is a, c, g, or t
nscyhndggshnnn

SEQ ID NO: 72 - DNA, 16 bp, Artificial Sequence
  /note=“Description of Artificial Sequence Synthetic EWS-FLI1 motif sequence”
aaggaaggaaggaagg

SEQ ID NO: 73 - DNA, 11 bp, Artificial Sequence
  /note=“Description of Artificial Sequence Synthetic control MSC motif sequence”
  misc_feature (1)..(1): n is a, c, g, or t
  misc_feature (2)..(2): n is a, c, g, or t
  misc_feature (4)..(4): k is g or t
  misc_feature (6)..(6): n is a, c, g, or t
  misc_feature (8)..(8): m is a or c
  misc_feature (10)..(10): n is a, c, g, or t
  misc_feature (11)..(11): n is a, c, g, or t
nntkantmann

SEQ ID NO: 74 - DNA, 14 bp, Artificial Sequence
  /note=“Description of Artificial Sequence Synthetic EWS-FLI1 MSC sequence”
aggaaggaaggaag

SEQ ID NO: 75 - protein, 264 aa, Homo sapiens; identical to SEQ ID NO: 3

SEQ ID NO: 76 - DNA, 16 bp, Artificial Sequence
  /note=“Description of Artificial Sequence Synthetic EWS-FLI1 binding sequence sequence”
ggaaggaaggaaggaa